(19) United States
(12) Patent Application Publication
(10) Pub. No.: US 2015/0293076 A1
(43) Pub. Date: Oct. 15, 2015

(54) Cellular Reprogramming for Product Optimization

(71) Applicant: Bolt Threads, Inc., Emeryville, CA (US)

(72) Inventors: Daniel Widmaier, San Francisco, CA (US); David Breslauer, Oakland, CA (US)

(21) Appl. No.: 14/437,476

(22) PCT Filed: Oct. 22, 2013
(86) PCT No.: PCT/US13/66159

§ 371 Date: Apr. 21, 2015

Related U.S. Application Data
(60) Provisional application No. 61/716,890, filed on Oct. 22, 2012.

(51) Int. Cl. G01N 33/50; C07K 14/00; C07K 14/435

(52) U.S. Cl. CPC G01N 33/5008; C07K 14/43518; C07K 14/001; C07K 2319/60

(57) ABSTRACT

The present disclosure identifies methods and compositions for modifying organisms, such that the organisms are optimized to produce or are enhanced to produce proteins or metabolites from cells. The present disclosure relates to methods of strain optimization to produce or enhance production of proteins or metabolites from cells. The present disclosure also relates to compositions resulting from those methods.

### CROSS-REFERENCE TO RELATED APPLICATIONS

This application claims the benefit of U.S. Provisional Application No. 61/716,890, filed Oct. 22, 2012, the disclosure of which is incorporated herein by reference.

### GOVERNMENT RIGHTS

This invention was made with government support under Army Research Office Grant W911-NF-10-1-0169. The government has certain rights in the invention.

### FIELD OF THE INVENTION

The present disclosure relates to methods of strain optimization to produce or enhance production of proteins or metabolites from cells. The present disclosure also relates to compositions resulting from those methods.

### BACKGROUND OF THE INVENTION

When producing proteins or metabolites from cells, a series of bottlenecks arise in various processes ranging from gene transcription, protein translation, post translational modification, secretion, metabolic flux of reaction components, to side product production/inhibition. Finding and alleviating these bottlenecks in series to improve the production of a desired product is a complicated and time-consuming process.

The current state of the art for solving this problem includes several methods to enhance production of proteins or metabolites, including gene knockouts, random DNA mutagenesis, global transcriptome factor mutagenesis, and gene overexpression. Gene knockouts lead to a large variation in presence or absence of a gene product within the cell. The all-or-none nature of this approach usually leads to cells with deficiencies in growth and metabolism. There is also no way to generate an adaptive response to the current metabolic state of the cell (e.g., the effect is constitutive). Random DNA mutagenesis creates random DNA mutations that can result in very large library sizes (depending upon how many bases are mutated and how large the genome size of the organism is). This requires the ability to search a vast library for phenotypes. In global transcription factor mutagenesis, a single transcription factor is mutated to generate a library, and is over-expressed in a cell to screen for a desired phenotype. This can generate large library sizes and is limited to the effects of one transcription factor. Finally, in gene overexpression, genes are selected for overexpression in a cell to perturb its activity, with the goal of an improved production phenotype. This process usually focuses on using a small number of well-characterized promoters to drive a library of target genes. This process doesn't easily allow for simultaneously screening large libraries with graded expression levels and allowing dynamic feedback processes to emerge.

What is needed therefore is a method to alter the production of proteins or metabolites by creating large perturbations in the metabolic state of the cell without requiring exceedingly large library sizes.

### SUMMARY OF THE INVENTION

Disclosed herein is a method to create large perturbations in the metabolic state of the cell by altering its signaling networks. In an embodiment, the invention provides a fusion of a library of promoters to a library of genes encoding regulatory elements such as regulatory proteins or regulatory RNAs. This combination leads to the possibility of large alterations of the cell's metabolic, regulatory, and signaling processes while also allowing for novel and altered dynamic timing and feedback mechanisms. In another embodiment, the changes to global expression are contained within relatively small library sizes (fewer than 100,000 members) allowing for a large search space with low screening needs to optimize the cell for the production and processing of proteins or metabolites. In an embodiment, the invention provides a fusion product between a random promoter and a random signaling protein. This method may be used to optimize strains through wide scale signaling disruption in cells of any type. This method may also provide a large search space for improved production of protein or metabolites.

In an embodiment a method of identifying a cell comprising an optimized functionality is provided, the method comprising obtaining a population of cells, wherein the population comprises cells engineered to include a member of an expression cassette library, wherein the expression cassette library comprises N distinct promoter elements, and M distinct regulatory elements, and wherein the library comprises up to (N×M) distinct combinations of the promoter elements operably linked to the regulatory elements, wherein each member of the expression cassette library comprises at least one of the N promoter elements operably linked to at least one of the M regulatory elements; and screening the population of cells to identify the cell comprising the optimized functionality.

In an embodiment, the identified cell further comprises a recombinant gene operably linked to a promoter. In an embodiment, the promoter is an inducible promoter. In an embodiment, the inducible promoter is induced by methanol. In an embodiment, the inducible promoter is AOX1 or AOX2. In another embodiment, the promoter is a constitutive promoter, such as a GAP promoter or a GCW14 promoter.

In an embodiment, the recombinant gene encodes a silk protein. In other embodiments, the recombinant gene encodes a protein fused to a detectable marker. In certain embodiments, the detectable marker is an epitope tag, a fluorescent protein, a firefly luciferase, or a beta galactosidase.

In some embodiments, the cell comprising the optimized functionality comprises a silk protein expressing gene operably linked to a recombinant AOX1 promoter. In other embodiments, the optimized functionality comprises an altered metabolic, regulatory, or signaling process in the cell comprising the optimized functionality as compared to an initial population of cells lacking the expression cassette. In still other embodiments, the optimized functionality comprises an increase in an expression level of a protein in the cell comprising the optimized functionality as compared to an expression level of the protein in an otherwise identical cell lacking the expression cassette. In yet other embodiments, the optimized functionality comprises an increase in a secretion level of a protein from the cell as compared to a secretion level of the protein from an otherwise identical cell lacking the expression cassette. In other embodiments, the optimized functionality comprises an alteration in the processing of a protein in the cell as compared to the processing of the protein in an otherwise identical cell lacking the expression cassette. In an embodiment, the protein is under the control of a recombinant AOX1 promoter. In an embodiment, the protein is a recombinant protein. In an embodiment, the protein is a silk protein. In some embodiments, the silk protein is a Major Ampullate Spidroin, Minor Ampullate Spidroin, Flagelliform Spidroin, Aciniform Spidroin, Pyriform Spidroin, Aggregate Spidroin, Tubuliform Spidroin, or Silkworm Fibroin.

In an embodiment, the optimized functionality comprises an increase in total production of a metabolite by the cell as compared to total production of a metabolite in an otherwise identical cell lacking the expression cassette. In certain embodiments, the metabolite is a farnasene, terpenoid, butanediol, propanediol, (+)-nootkatone, or carotenoid. In some embodiments the metabolite is formic acid, methanol, carbon monoxide, carbon dioxide, syngas, acetaldehyde, acetic acid, anhydride, ethanol, glycine, oxalic acid, ethylene glycol, ethylene oxide, alanine, glycerol, 3-hydroxypropionic acid, lacitic acid, malonic acid, serine, propionic acid, acetone, acetoin, aspartic acid, butanol, fumaric acid, 3-hydroxybutyroloactone, malic acid, succinic acid, threonine, arabinitol, furfural, glutamic acid, glutaric acid, itaconic acid, levulinic acid, proline, xylitol, xylonic acid, aconitic acid, adipic acid, ascorbic acid, citric acid, fructose, 2,5-furan dicarboxylic acid, glucaric acid, gluconic acid, kojic acid, comeric acid, lysine, or sorbitol. In certain embodiments, the metabolite is fatty acid methyl ester, alkane, bio-oil, green crude, lactic acid, isobutanol, squalane, 1,4-butanediol, butadiene, acrylamide, isobutene, methionine, I-methionine, glutamate, 1,3-propanediol, mandelic acid, vanillin, valencene, isoprene, polybutylene succinate, or modified polybutylene succinate.

In an embodiment, the cells are prokaryotes. In a further embodiment, the prokaryotes are from the species Escherichia coli, Salmonella enterica, Bacillus subtilis, or Streptomyces. In an embodiment, the prokaryote is Escherichia coli. In another embodiment, the cells are yeast cells. In some embodiments, the yeast cells are of the species Pichia (Komagataella) pastoris, Hansenula polymorphs, Arxula adeninivorans, Yarrowia lipolytica, Pichia (Scheffersomyces) stipitis, Pichia methanolica, Saccharomyces cerevisiae, or Kluyveromyces lactis. In an embodiment, the yeast cells are from the strain Pichia (Komagataella) pastoris.

In an embodiment, the N distinct promoter elements consist of all known promoter elements endogenous to the cell. In an embodiment, the N distinct promoter elements consist of a subset of all known promoter elements endogenous to the cell. In an embodiment, the N distinct promoter elements comprise a subset of all known promoter elements endogenous to the cell. In an embodiment, the N distinct promoter elements comprise promoter elements exogenous to said cell. In an embodiment, the N distinct promoter elements comprise synthetic promoter elements. In an embodiment, the M distinct regulatory elements consist of all known regulatory elements endogenous to the cell. In an embodiment, the M distinct regulatory elements consist of a subset of all known regulatory elements endogenous to the cell. In an embodiment, the M distinct regulatory elements comprise a subset of all known regulatory elements endogenous to the cell. In an embodiment, the M distinct regulatory elements comprise regulatory elements exogenous to the cell. In an embodiment, the M distinct regulatory elements comprise synthetic regulatory elements.

In an embodiment, the promoter element is a chimeric promoter element. In certain embodiments, the regulatory element is selected from Table 1. In an embodiment, the regulatory element is heterologous to the cell. In an embodiment, the regulatory element comprises a transcription factor. In another embodiment, the regulatory element comprises a signaling protein. In another embodiment, the regulatory element comprises a regulatory RNA element. In certain embodiments, the regulatory RNA element is a microRNA. In other embodiments, the regulatory RNA element is an antisense RNA. In yet other embodiments, the regulatory RNA element is an aptamer.

In an embodiment, N is less than 10,000. In another embodiment, N is less than 6,000. In another embodiment, M is less than 1,000. In still another embodiment, M is less than 500. In yet another embodiment, (N×M) is less than 2 million.

In an embodiment, the expression cassette member further comprises a replication origin. In another embodiment, the expression cassette member further comprises a selection marker. In still another embodiment, the expression cassette member further comprises a replication origin and a selection marker. In yet another embodiment, the expression cassette is a linear fragment that is incorporated into the cell's chromosome.

In some embodiments, the screening comprises selecting on a selective media the cell comprising the optimized functionality. In some embodiments, the media is selective for auxotrophy or an antibiotic resistance marker.

In an embodiment, the method of identifying a cell comprising an optimized functionality further comprises isolating the cell comprising the optimized functionality. In an embodiment, the population of cells were previously identified as comprising an optimized functionality using the method of identifying a cell comprising an optimized functionality.

Also provided herein is a library of expression cassettes, wherein the expression cassette library comprises N distinct promoter elements, and M distinct regulatory elements, and wherein the library comprises up to (N×M) distinct combinations of the promoter elements operably linked to the regulatory elements, wherein each member of the expression cassette library comprises at least one of the N promoter elements operably linked to at least one of the M regulatory elements.

In an embodiment, the promoter element is a chimeric promoter element. In an embodiment, the regulatory element is selected from Table 1. In an embodiment, the regulatory element is heterologous to the cell. In an embodiment, the regulatory element comprises a transcription factor. In other embodiments, the regulatory element comprises a signaling protein. In other embodiments, the regulatory element comprises a regulatory RNA element. In certain embodiments, the regulatory RNA element is a microRNA. In other embodiments, the regulatory RNA element is an antisense RNA. In yet other embodiments, the regulatory RNA element is an aptamer.

In an embodiment, N is less than 10,000. In other embodiments, N is less than 6,000. In other embodiments, M is less than 1,000. In still other embodiments, M is less than 500. In yet other embodiments, (N×M) is less than 2 million.

In an embodiment, the expression cassette member further comprises a replication origin. In an embodiment, the expression cassette member further comprises a selection marker. In an embodiment, the expression cassette member further comprises a replication origin and a selection marker. In an embodiment, the expression cassette is a linear fragment that is incorporated into the cell's chromosome.

Also provided herein, are embodiments comprising a library of cells wherein each cell in the library of cells is engineered to include a member of an expression cassette library, wherein the expression cassette library comprises N distinct promoter elements, and M distinct regulatory elements, and wherein the library comprises up to (N×M) distinct combinations of the promoter elements operably linked to the regulatory elements, wherein each member of the expression cassette library comprises at least one of the N promoter elements operably linked to at least one of the M regulatory elements.

In certain embodiments, the cells are prokaryotes. In certain embodiments, the prokaryotes are from the species Escherichia coli, Salmonella enterica, Bacillus subtilis, or Streptomyces. In an embodiment, the prokaryote is Streptomyces. In another embodiment, the cells are yeast cells. In an embodiment, the yeast cells are of the species Pichia (Komagataella) pastoris, Hansenula polymorphs, Arxula adeninivorans, Yarrowia lipolytica, Pichia (Scheffersomyces) stipitis, Pichia methanolica, Saccharomyces cerevisiae, or Kluyveromyces lactis. In an embodiment, the yeast cells are from the strain Pichia (Komagataella) pastoris.

In an embodiment, the promoter element is a chimeric promoter element. In an embodiment, the regulatory element is selected from Table 1. In an embodiment, the regulatory element is heterologous to the cell. In an embodiment, the regulatory element comprises a transcription factor. In another embodiment, the regulatory element comprises a signaling protein. In another embodiment, the regulatory element comprises a regulatory RNA element. In an aspect, the regulatory RNA element is a microRNA. In another embodiment, the regulatory RNA element is an antisense RNA. In yet another embodiment, the regulatory RNA element is an aptamer.

In an embodiment, N is less than 10,000. In another embodiment, N is less than 6,000. In another embodiment, M is less than 1,000. In still another embodiment, M is less than 500. In yet another embodiment, (N×M) is less than 2 million.

In an embodiment, the expression cassette member further comprises a replication origin. In an embodiment, the expression cassette member further comprises a selection marker. In an embodiment, the expression cassette member further comprises a replication origin and a selection marker. In yet another embodiment, the expression cassette is a linear fragment that is incorporated into the cell's chromosome.

Also provided herein, in one aspect, is a method of engineering a host cell to acquire an optimized functionality, comprising: introducing an expression cassette into the host cell, wherein the expression cassette comprises a promoter element operably linked to a regulatory element; and expressing the regulatory element within the host cell, wherein expression of the regulatory element results in an engineered host cell having an optimized functionality as compared to an otherwise identical cell lacking the expression cassette.

In an embodiment, the combination of the promoter element operably linked to the regulatory element is not native to the host cell. In an embodiment, the expression cassette was identified using the method of identifying a cell comprising an optimized functionality, as disclosed herein. In an embodiment, the combination of the promoter element operably linked to the regulatory element was previously identified by a third party.

Also provided herein is an embodiment comprising a method of engineering a host cell to acquire an optimized functionality, comprising: identifying from a population of modified host cells at least one modified host cell comprising the optimized functionality, wherein each of the modified host cells is engineered to include a member of an expression cassette library, wherein the expression cassette library comprises N distinct promoter elements, and M distinct regulatory elements, and wherein the library comprises up to (N×M) distinct combinations of the promoter elements operably linked to the regulatory elements, wherein each member of the expression cassette library comprises at least one of the N promoter elements operably linked to at least one of the M regulatory elements, and wherein the population of modified host cells is screened to identify a modified host cell comprising the optimized functionality; comparing RNA expression in the modified host cell comprising the optimized functionality with RNA expression in an otherwise identical host cell lacking the member of the expression cassette library to identify an RNA transcript whose expression significantly differs between the modified host cell comprising the optimized functionality and the host cell lacking the member of the expression cassette library; and engineering the host cell lacking the member to adjust the direction of the expression level of the identified RNA transcript toward the level found in the modified host cell comprising the optimized functionality, wherein the engineered cell does not comprise the member of the expression cassette library.

In an embodiment, the modification of the host cell comprises increasing expression levels of the at least one selected gene. In another embodiment, the modification of the host cell comprises decreasing expression levels of the at least one selected gene. In another embodiment aspect, the modification of the host cell comprises knocking out the at least one selected gene.

These and other embodiments of the invention are further described in the Figures, Description, Examples and Claims, herein.

### BRIEF DESCRIPTION OF THE FIGURES

FIG. 1 shows an exemplary method of selecting promoter-regulatory element pairs and assembling them into vectors (e.g., by ligation, chew-back and anneal (e.g., Gibson), recombination, or mating). Assembled vectors are transformed or mated into the selected cell for downstream screening.

FIG. 2 shows steps for isolating specific changes to cellular metabolism from improved strains.

FIG. 3 depicts a Pichia cell transformed with the library of promoter-TF combinations and a silk protein with a reporter under AOX1 control.

FIG. 4 depicts histograms showing the normalized variation of manual and robotic pipetting.

FIG. 5 shows the normalized variability of Bradford and BCA assays for samples of known initial protein concentrate.

FIG. 6 shows the normalized fluorescence variability between wells across four quadrants of one plate.

FIG. 7 shows, in order of descending initial cell concentration from top to bottom: fluorescence and optical density for each quadrant of a single plate expressing fluorescent protein. On left: fluorescence vs. optical density for each well within a quadrant. On right: kernel densities fit to normalized fluorescence per optical density for wells within a quadrant.

FIG. 8 shows cell growth in stacked 96-well plates, comparing plate types, gap size between plates, and growth on top of or bottom of a stack of plates. Thick lines signify plates' cell densities after two days of growth; black lines represent data from experiments where two plate spacers separated the stacked plates, and grey lines represent data from experiments where one plate spacer separated the stacked plates.

FIG. 9 shows the composition of plasmid RM963, which expresses the genes necessary for production of lycopene in Pichia pastoris.

FIG. 10 presents the absorbance spectrum of an ethyl acetate extract from a Pichia pastoris strain producing lycopene.

FIG. 11 illustrates a process for generating a library of promoters operably linked to regulatory elements.

FIG. 12 depicts the differences in lycopene production before and after introduction of library members in Pichia pastoris.

FIG. 13 shows the composition of a silk-GFP expression cassette.

FIG. 14 presents a western blot analysis of a silk-GFP secreting strain of Pichia pastoris.

FIG. 15 shows the fluorescence of secreted proteins before and after introduction of library members in Pichia pastoris.

FIG. 16 depicts the composition of plasmid RM991, which expresses intracellular GFP in Saccharomyces cerevisiae.

FIG. 17 shows the composition of a promoter-regulatory element library in a vector suitable for transformation into Saccharomyces cerevisiae.

FIG. 18 shows the fluorescence of cells before and after introduction of library members in Saccharomyces cerevisiae.

### DETAILED DESCRIPTION

Described in this specification is a process including the steps of genetically perturbating a collection of cells and screening the perturbed cells for altered (e.g., improved) production of a product. In certain embodiments the process relies on the cell's own promoters and regulatory elements to “reprogram” the cell's internal control network, advantageously limiting the number of different perturbations to a quantity that can be conveniently physically screened for phenotype without sacrificing the desired improvement in product production.

In a cell, regulatory elements, including by way of example but not limitation, regulatory proteins (e.g., transcription factors), chaperones, signaling proteins, RNAi molecules, antisense RNA molecules, microRNAs and RNA aptamers, control the transcriptional activation of promoters and other cellular signaling mechanisms. This control can be both positive (increasing expression) and negative (decreasing expression). In addition, a single regulatory element may control many other cellular components, many of which may also be regulatory elements, creating a cascade effect in the cellular control circuitry. Since we don't know a priori which of these effects is likely to result in increased product production, random expression of regulatory elements provides good way to generate many different cellular changes using the fewest number of initial effectors.

However, simply expressing the regulatory elements may not be sufficient to achieve a desired level of product. If an element is expressed at the wrong time, or at the wrong strength it may be toxic to the cell. However, if expressed correctly it may improve product production. In addition, an ideal system may involve feedback. For example, it may be useful to express the regulatory element for a selected amount of time, and then stop expression. These feedback mechanisms are often integrated at the promoters of genes as a site of transcriptional feedback control. Therefore, by generating combinations of regulatory elements with promoters, many combinations of regulatory reprogramming are achieved which may affect, for example, timing of metabolite or protein expression, magnitude of induction, and feedback control processes. By screening cells to identify those having a desired regulatory reprogramming combinations, this process provides enhanced likelihood of finding perturbations that greatly improve product production within any given library size. The same principles can be used to enhance the likelihood of finding optimal combinations of regulatory elements and promoters using subsets of the total number of endogenous regulatory element and promoter combinations as well as combinations generated using exogenous, or synthetic regulatory elements and promoters.

### I. DEFINITIONS

Unless otherwise defined herein, scientific and technical terms used in connection with the present invention shall have the meanings that are commonly understood by those of ordinary skill in the art. Further, unless otherwise required by context, singular terms shall include the plural and plural terms shall include the singular. The terms “a” and “an” includes plural references unless the context dictates otherwise. Generally, nomenclatures used in connection with, and techniques of, biochemistry, enzymology, molecular and cellular biology, microbiology, genetics and protein and nucleic acid chemistry and hybridization described herein are those well-known and commonly used in the art.

The following terms, unless otherwise indicated, shall be understood to have the following meanings:

The term “polynucleotide” or “nucleic acid molecule” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native internucleoside bonds, or both. The nucleic acid can be in any topological conformation. For instance, the nucleic acid can be single-stranded, double-stranded, triple-stranded, quadruplexed, partially double-stranded, branched, hairpinned, circular, or in a padlocked conformation.

Unless otherwise indicated, and as an example for all sequences described herein under the general format “SEQ ID NO:”, “nucleic acid comprising SEQ ID NO:1” refers to a nucleic acid, at least a portion of which has either (i) the sequence of SEQ ID NO:1, or (ii) a sequence complementary to SEQ ID NO:1. The choice between the two is dictated by the context. For instance, if the nucleic acid is used as a probe, the choice between the two is dictated by the requirement that the probe be complementary to the desired target.

An “isolated” RNA, DNA or a mixed polymer is one which is substantially separated from other cellular components that naturally accompany the native polynucleotide in its natural host cell, e.g., ribosomes, polymerases and genomic sequences with which it is naturally associated.

An “isolated” organic molecule (e.g., a silk protein) is one which is substantially separated from the cellular components (membrane lipids, chromosomes, proteins) of the host cell from which it originated, or from the medium in which the host cell was cultured. The term does not require that the biomolecule has been separated from all other chemicals, although certain isolated biomolecules may be purified to near homogeneity.

The term “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids.

An endogenous nucleic acid sequence in the genome of an organism (or the encoded protein product of that sequence) is deemed “recombinant” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). By way of example, a promoter sequence can be substituted (e.g., by homologous recombination) for the native promoter of a gene in the genome of a host cell, such that this gene has an altered expression pattern. This gene would now become “recombinant” because it is separated from at least some of the sequences that naturally flank it.

A nucleic acid is also considered “recombinant” if it contains any modifications that do not naturally occur to the corresponding nucleic acid in a genome. For instance, an endogenous coding sequence is considered “recombinant” if it contains an insertion, deletion or a point mutation introduced artificially, e.g., by human intervention. A “recombinant nucleic acid” also includes a nucleic acid integrated into a host cell chromosome at a heterologous site and a nucleic acid construct present as an episome.

As used herein, the phrase “degenerate variant” of a reference nucleic acid sequence encompasses nucleic acid sequences that can be translated, according to the standard genetic code, to provide an amino acid sequence identical to that translated from the reference nucleic acid sequence. The term “degenerate oligonucleotide” or “degenerate primer” is used to signify an oligonucleotide capable of hybridizing with target nucleic acid sequences that are not necessarily identical in sequence but that are homologous to one another within one or more particular segments.

The term “percent sequence identity” or “identical” in the context of nucleic acid sequences refers to the residues in the two sequences which are the same when aligned for maximum correspondence. The length of sequence identity comparison may be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA provides alignments and percent sequence identity of the regions of the best overlap between the query and search sequences. Pearson, Methods Enzymol. 183:63-98 (1990) (hereby incorporated by reference in its entirety). For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference. Alternatively, sequences can be compared using the computer program, BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

The term “substantial homology” or “substantial similarity,” when referring to a nucleic acid or fragment thereof, indicates that, when optimally aligned with appropriate nucleotide insertions or deletions with another nucleic acid (or its complementary strand), there is nucleotide sequence identity in at least about 75%, 80%, 85%, preferably at least about 90%, and more preferably at least about 95%, 96%, 97%, 98% or 99% of the nucleotide bases, as measured by any well-known algorithm of sequence identity, such as FASTA, BLAST or Gap, as discussed above.

Alternatively, substantial homology or similarity exists when a nucleic acid or fragment thereof hybridizes to another nucleic acid, to a strand of another nucleic acid, or to the complementary strand thereof, under stringent hybridization conditions. “Stringent hybridization conditions” and “stringent wash conditions” in the context of nucleic acid hybridization experiments depend upon a number of different physical parameters. Nucleic acid hybridization will be affected by such conditions as salt concentration, temperature, solvents, the base composition of the hybridizing species, length of the complementary regions, and the number of nucleotide base mismatches between the hybridizing nucleic acids, as will be readily appreciated by those skilled in the art. One having ordinary skill in the art knows how to vary these parameters to achieve a particular stringency of hybridization.

In general, “stringent hybridization” is performed at about 25° C. below the thermal melting point (Tₘ) for the specific DNA hybrid under a particular set of conditions. “Stringent washing” is performed at temperatures about 5° C. lower than the Tₘ for the specific DNA hybrid under a particular set of conditions. The Tₘ is the temperature at which 50% of the target sequence hybridizes to a perfectly matched probe. See Sambrook et al., Molecular Cloning: A Laboratory Manual, 2d ed., Cold Spring Harbor Laboratory Press, Cold Spring Harbor, N.Y. (1989), page 9.51, hereby incorporated by reference. For purposes herein, “stringent conditions” are defined for solution phase hybridization as aqueous hybridization (i.e., free of formamide) in 6×SSC (where 20×SSC contains 3.0 M NaCl and 0.3 M sodium citrate), 1% SDS at 65° C. for 8-12 hours, followed by two washes in 0.2×SSC, 0.1% SDS at 65° C. for 20 minutes. It will be appreciated by the skilled worker that hybridization at 65° C. will occur at different rates depending on a number of factors including the length and percent identity of the sequences which are hybridizing.

The nucleic acids (also referred to as polynucleotides) of this present invention may include both sense and antisense strands of RNA, cDNA, genomic DNA, and synthetic forms and mixed polymers of the above. They may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.) Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule. Other modifications can include, for example, analogs in which the ribose ring contains a bridging moiety or other structure such as the modifications found in “locked” nucleic acids.

The term “mutated” when applied to nucleic acid sequences means that nucleotides in a nucleic acid sequence may be inserted, deleted or changed compared to a reference nucleic acid sequence. A single alteration may be made at a locus (a point mutation) or multiple nucleotides may be inserted, deleted or changed at a single locus. In addition, one or more alterations may be made at any number of loci within a nucleic acid sequence. A nucleic acid sequence may be mutated by any method known in the art including but not limited to mutagenesis techniques such as “error-prone PCR” (a process for performing PCR under conditions where the copying fidelity of the DNA polymerase is low, such that a high rate of point mutations is obtained along the entire length of the PCR product; see, e.g., Leung et al., Technique, 1:11-15 (1989) and Caldwell and Joyce, PCR Methods Applic. 2:28-33 (1992)); and “oligonucleotide-directed mutagenesis” (a process which enables the generation of site-specific mutations in any cloned DNA segment of interest; see, e.g., Reidhaar-Olson and Sauer, Science 241:53-57 (1988)).

The term “attenuate” as used herein generally refers to a functional deletion, including a mutation, partial or complete deletion, insertion, or other variation made to a gene sequence or a sequence controlling the transcription of a gene sequence, which reduces or inhibits production of the gene product, or renders the gene product non-functional. In some instances a functional deletion is described as a knockout mutation. Attenuation also includes amino acid sequence changes by altering the nucleic acid sequence, placing the gene under the control of a less active promoter, down-regulation, expressing interfering RNA, ribozymes or antisense sequences that target the gene of interest, or through any other technique known in the art. In one example, the sensitivity of a particular enzyme to feedback inhibition or inhibition caused by a composition that is not a product or a reactant (non-pathway specific feedback) is lessened such that the enzyme activity is not impacted by the presence of a compound. In other instances, an enzyme that has been altered to be less active can be referred to as attenuated.

The term “deletion” as used herein refers to the removal of one or more nucleotides from a nucleic acid molecule or one or more amino acids from a protein, the regions on either side being joined together.

The term “knock-out” as used herein is intended to refer to a gene whose level of expression or activity has been reduced to zero. In some examples, a gene is knocked-out via deletion of some or all of its coding sequence. In other examples, a gene is knocked-out via introduction of one or more nucleotides into its open reading frame, which results in translation of a non-sense or otherwise non-functional protein product.

The term “vector” as used herein is intended to refer to a nucleic acid molecule capable of transporting another nucleic acid to which it has been linked. One type of vector is a “plasmid,” which generally refers to a circular double stranded DNA loop into which additional DNA segments may be ligated, but also includes linear double-stranded molecules such as those resulting from amplification by the polymerase chain reaction (PCR) or from treatment of a circular plasmid with a restriction enzyme. Other vectors include cosmids, bacterial artificial chromosomes (BAC) and yeast artificial chromosomes (YAC). Another type of vector is a viral vector, wherein additional DNA segments may be ligated into the viral genome (discussed in more detail below). Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain preferred vectors are capable of directing the expression of genes to which they are operatively linked. Such vectors are referred to herein as “recombinant expression vectors” (or simply “expression vectors”).

“Operatively linked” or “operably linked” expression control sequences refers to a linkage in which the expression control sequence is contiguous with the gene of interest to control the gene of interest, as well as expression control sequences that act in trans or at a distance to control the gene of interest.

The term “expression control sequence” refers to polynucleotide sequences which are necessary to affect the expression of coding sequences to which they are operatively linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

The term “regulatory element” refers to any element which affects transcription or translation of a nucleic acid molecule. These include, by way of example but not limitation: regulatory proteins (e.g., transcription factors), chaperones, signaling proteins, RNAi molecules, antisense RNA molecules, microRNAs and RNA aptamers. Regulatory elements may be endogenous to the host organism. Regulatory elements may also be exogenous to the host organism. Regulatory elements may be synthetically generated regulatory elements.

The term “promoter,” “promoter element,” or “promoter sequence” as used herein, refers to a DNA sequence which when ligated to a nucleotide sequence of interest is capable of controlling the transcription of the nucleotide sequence of interest into mRNA. A promoter is typically, though not necessarily, located 5′ (i.e., upstream) of a nucleotide sequence of interest whose transcription into mRNA it controls, and provides a site for specific binding by RNA polymerase and other transcription factors for initiation of transcription. Promoters may be endogenous to the host organism. Promoters may also be exogenous to the host organism. Promoters may be synthetically generated regulatory elements.

Promoters useful for expressing the recombinant genes described herein include both constitutive and inducible/repressible promoters. Where multiple recombinant genes are expressed in an engineered organism of the invention, the different genes can be controlled by different promoters or by identical promoters in separate operons, or the expression of two or more genes may be controlled by a single promoter as part of an operon.

The term “recombinant host cell” (or simply “host cell”), as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism.

The term “peptide” as used herein refers to a short polypeptide, e.g., one that is typically less than about 50 amino acids long and more typically less than about 30 amino acids long. The term as used herein encompasses analogs and mimetics that mimic structural and thus biological function.

The term “polypeptide” encompasses both naturally-occurring and non-naturally-occurring proteins, and fragments, mutants, derivatives and analogs thereof. A polypeptide may be monomeric or polymeric. Further, a polypeptide may comprise a number of different domains each of which has one or more distinct activities.

The term “isolated protein” or “isolated polypeptide” is a protein or polypeptide that by virtue of its origin or source of derivation (1) is not associated with naturally associated components that accompany it in its native state, (2) exists in a purity not found in nature, where purity can be adjudged with respect to the presence of other cellular material (e.g., is free of other proteins from the same species) (3) is expressed by a cell from a different species, or (4) does not occur in nature (e.g., it is a fragment of a polypeptide found in nature or it includes amino acid analogs or derivatives not found in nature or linkages other than standard peptide bonds). Thus, a polypeptide that is chemically synthesized or synthesized in a cellular system different from the cell from which it naturally originates will be “isolated” from its naturally associated components. A polypeptide or protein may also be rendered substantially free of naturally associated components by isolation, using protein purification techniques well known in the art. As thus defined, “isolated” does not necessarily require that the protein, polypeptide, peptide or oligopeptide so described has been physically removed from its native environment.

The term “polypeptide fragment” refers to a polypeptide that has a deletion, e.g., an amino-terminal and/or carboxy-terminal deletion compared to a full-length polypeptide. In a preferred embodiment, the polypeptide fragment is a contiguous sequence in which the amino acid sequence of the fragment is identical to the corresponding positions in the naturally-occurring sequence. Fragments typically are at least 5, 6, 7, 8, 9 or 10 amino acids long, preferably at least 12, 14, 16 or 18 amino acids long, more preferably at least 20 amino acids long, more preferably at least 25, 30, 35, 40 or 45, amino acids, even more preferably at least 50 or 60 amino acids long, and even more preferably at least 70 amino acids long.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences.) As used herein, homology between two regions of amino acid sequence (especially with respect to predicted structural similarities) is interpreted as implying similarity in function.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art. See, e.g., Pearson, 1994, Methods Mol. Biol. 24:307-31 and 25:365-89 (herein incorporated by reference).

The twenty conventional amino acids and their abbreviations follow conventional usage. See Immunology-A Synthesis (Golub and Gren eds., Sinauer Associates, Sunderland, Mass., 2^{nd} ed. 1991), which is incorporated herein by reference. Stereoisomers (e.g., D-amino acids) of the twenty conventional amino acids, unnatural amino acids such as α-, α-disubstituted amino acids, N-alkyl amino acids, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, N-methylarginine, and other similar amino acids and imino acids (e.g., 4-hydroxyproline). In the polypeptide notation used herein, the left-hand end corresponds to the amino terminal end and the right-hand end corresponds to the carboxy-terminal end, in accordance with standard usage and convention.

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for polypeptides, which is sometimes also referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using a measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild-type protein and a mutein thereof. See, e.g., GCG Version 6.1.

A useful algorithm when comparing a particular polypeptide sequence to a database containing a large number of sequences from different organisms is the computer program BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

Preferred parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

Preferred parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62. The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences. Pearson, Methods Enzymol. 183:63-98 (1990) (incorporated by reference herein). For example, percent sequence identity between amino acid sequences can be determined using FASTA with its default parameters (a word size of 2 and the PAM250 scoring matrix), as provided in GCG Version 6.1, herein incorporated by reference.

The term “region” refers to a physically contiguous portion of the primary structure of a biomolecule. In the case of proteins, a region is defined by a contiguous portion of the amino acid sequence of that protein.

The term “domain” refers to a structure of a biomolecule that contributes to a known or suspected function of the biomolecule. Domains may be co-extensive with regions or portions thereof; domains may also include distinct, non-contiguous regions of a biomolecule. Examples of protein domains include, but are not limited to, an Ig domain, an extracellular domain, a transmembrane domain, and a cytoplasmic domain.

The term “metabolite” refers to any substance produced or used during all the physical and chemical processes within a cell that create and use energy. The term “metabolic precursors” refers to compounds from which the metabolites are made. The term “metabolic products” refers to any substance that is part of a metabolic pathway (e.g., metabolite, metabolic precursor).

Throughout this specification and claims, the word “comprise” or variations such as “comprises” or “comprising,” will be understood to imply the inclusion of a stated integer or group of integers but not the exclusion of any other integer or group of integers.

Exemplary methods and materials are described below, although methods and materials similar or equivalent to those described herein can also be used in the practice of the present invention and will be apparent to those of skill in the art. All publications and other references mentioned herein are incorporated by reference in their entirety. In case of conflict, the present specification, including definitions, will control. The materials, methods, and examples are illustrative only and not intended to be limiting.

### II. CELLULAR REPROGRAMMING

Described is a method to make random perturbations to a large number of cells and screen the population of cells for cells that improve product production. To narrow the number of different perturbations to a quantity that can be conveniently physically screened for phenotype without sacrificing scope—the process used herein relies on the cell's own promoters and regulatory elements in order to “reprogram” the cell's internal control network.

In a cell, regulatory elements, including by way of example but not limitation regulatory proteins (e.g., transcription factors), chaperones, signaling proteins, RNAi molecules, antisense RNA molecules, microRNAs and RNA aptamers, control the transcriptional activation of promoters and other cellular signaling mechanisms. This control can be both positive (increasing expression) and negative (decreasing expression). In addition, a single regulatory element may control many other cellular components, many of which may also be regulatory elements, creating a cascade effect in the cellular control circuitry. Without wishing to be bound by theory, we hypothesize that a population of cells transformed with a library of regulatory element/promoter combinations produces many combinations of regulatory reprogramming with concomitant changes in transcription timing, magnitude of induction and feedback control. By screening cells harboring a library of a given size comprising these combinations, cells are identified having resulting perturbations that greatly improve a desirable cell characteristic, e.g., product production.

In an embodiment, a method is disclosed for reprogramming a cell to alter production of a desired product in a target cell. This product could be, for example, a protein or a metabolite. The method includes selecting a target cell type, and identifying a set of regulatory elements and promoter elements of the target cell type to create a library of promoter-regulatory element pairs wherein each regulatory element in the set is combined with each promoter set. In an embodiment, the set consists of all known regulatory elements and all known promoter elements endogenous to the target cell. In another embodiment, the set consists of a subset of all known regulatory elements and all known promoter elements endogenous to the target cell. In another embodiment the set consists of all known regulatory elements and a subset of known promoter elements endogenous to the target cell. In another embodiment, the set consists of a subset of all known regulatory elements and all known promoter elements endogenous to the target cell. In yet other embodiments, the library is created using exogenous and/or synthetic regulatory elements and/or promoters. The library of promoter-regulatory element pairs is introduced into the target cells, resulting in many combinations of regulatory reprogramming in the target cells which can affect, for example, regulatory timing, magnitude of induction, and feedback control processes. The cells are grown and clones containing unique library elements are isolated and screened for optimized regulatory reprogramming (via, e.g., desired product production). By screening cells for the desired regulatory reprogramming (e.g., improved protein or product expression), this process provides a high likelihood of finding perturbations that greatly improve product production using a given library size. Library elements that create the desired producing clones (depending on desired outcome) can be isolated and identified. Once identified, useful library elements can be introduced into other target cells (preferably of the same type) to drive production of other products. The process above or selected steps from the process above can optionally be repeated.

In this system of transforming or mating cells to contain random promoter—regulatory element pairs, the optimized product could be anything that is measureable from proteins to small molecules. While the majority of examples herein are proteins to optimize titer and secretion, the same could be applied to metabolite production or engineered metabolite production. Examples of this would include production of farnasene, terpenoids, butanediol, propanediol, (+)-nootkatone, or carotenoids. Other examples of metabolites include, but are not limited to, formic acid, methanol, carbon monoxide, carbon dioxide, syngas, acetaldehyde, acetic acid, anhydride, ethanol, glycine, oxalic acid, ethylene glycol, ethylene oxide, alanine, glycerol, 3-hydroxypropionic acid, lacitic acid, malonic acid, serine, propionic acid, acetone, acetoin, aspartic acid, butanol, fumaric acid, 3-hydroxybutyroloactone, malic acid, succinic acid, threonine, arabinitol, furfural, glutamic acid, glutaric acid, itaconic acid, levulinic acid, proline, xylitol, xylonic acid, aconitic acid, adipic acid, ascorbic acid, citric acid, fructose, 2,5-furan dicarboxylic acid, glucaric acid, gluconic acid, kojic acid, comeric acid, lysine, sorbitol, fatty acid methyl ester, alkane, bio-oil, green crude, lactic acid, isobutanol, squalane, 1,4-butanediol, butadiene, acrylamide, isobutene, methionine, I-methionine, glutamate, 1,3-propanediol, mandelic acid, vanillin, valencene, isoprene, polybutylene succinate, and modified polybutylene succinate. Other difficult proteins that may be expressed using the methods and compositions disclosed herein include proteins typified by one or more of the following: intrinsically unstructured, toxic to cells including host cells, highly repetitive, encoded by GC rich genes, function by embedding in lipid bilayer membranes, cause signaling events within the host cell, deplete pools of metabolites in host cells, are not properly trafficked through secretory pathways, are not properly post-translationally modified. A list of difficult proteins that may be expressed by the methods and compositions disclosed herein is found in Table 3 of Cereghino and Cregg, FEMS Microbiology Reviews, 20 (2000) 45-66. This list comprises nearly 200 proteins tried in Pichia and all could be improved by application of the method disclosed herein.

The target cell type is selected based on the type of product desired, the eventual production environment and cost considerations. Often an organism is chosen because it already contains a pathway similar to the desired production pathway, thus resulting less required alterations. The method described here will work, for example, with bacterial (e.g., E. coli), yeast (e.g., S. cerevisiae and P. pastoris) and higher eukaryotic cells. Other yeast expression systems can be used, for example, Hansenula polymorphs, Arxula adeninivorans, Yarrowia lipolytica, Pichia (Scheffersomyces) stipites, Pichia methanolica, Saccharomyces cerevisiae, or Kluyveromyces lactis. Filamentous fungi may also be used in an expression system described herein, for example, in Tricoderma reesei, Aspergillus, Sordaria macrospora, or Neurospora crassa.

A. Promoter and Regulatory Element Identification

In a preferred embodiment, all known and potential regulatory elements from the target cell type are identified. In other embodiments, a subset of known and potential regulatory elements from the target cell type are identified. Regulatory elements include, for example, regulatory proteins (e.g., transcription factors), chaperones, signaling proteins, RNAi molecules, antisense RNA molecules, microRNAs and RNA aptamers. In some organisms such as E. coli and S. cerevisiae many of these elements have been discovered and are annotated in genomic repositories such as Genbank. In other cases, these elements are not known, but can be discovered through bioinformatics prediction tools such as pfam. The resulting list of putative regulatory elements is sufficient for this method—the screening approach will automatically eliminate any elements that turn out to be non-regulatory. The result of this step is a list of DNA sequences for each known and putative regulatory element.

To identify regulatory element sequences, at least a part of the genomic sequence of the organism is required. A complete genomic sequence will yield the best results, but partial sequences may also be used. In some cases, product production may be enhanced using regulatory elements from a heterologous organism. Choice of the heterologous organism will depend on the specific situation. For example, if the product is created using heterologous genes taken from an organism that is different from the desired expression host organism, the library can include regulatory elements (and promoters—see below) from the original source organism. The use of regulatory elements from related species is preferred since important regulation (for the desired product) may exist in a related species. For example, some S. cerevisiae proteins are shown in the literature to improve function in P. pastoris beyond what overexpression of the native ortholog can achieve (Zhang, W., et al., Enhanced Secretion of Heterologous Proteins in Pichia pastoris Following Overexpression of Saccharomyces cerevisiae Chaperone Proteins. Biotechnology progress, 22(4), 1090-1095 (2006)).

Promoter elements are identified in the target cell type. This step is similar to identification of regulatory elements described above, but the goal is to identify known and putative promoter sequences. Unknown promoter sequences can be acquired by first using bioinformatics tools to identify predicted open reading frames in the organisms DNA. The DNA 5-prime to (preceding) the start codon in the open reading frame is the promoter, the exact length of the promoter in base pairs depends upon the organism. In bacteria, this region is typically few hundred bases long. In yeast, this region can be up to a few thousand bases. In higher eukaryotes, several thousand bases are typically necessary to capture the promoter sequence.

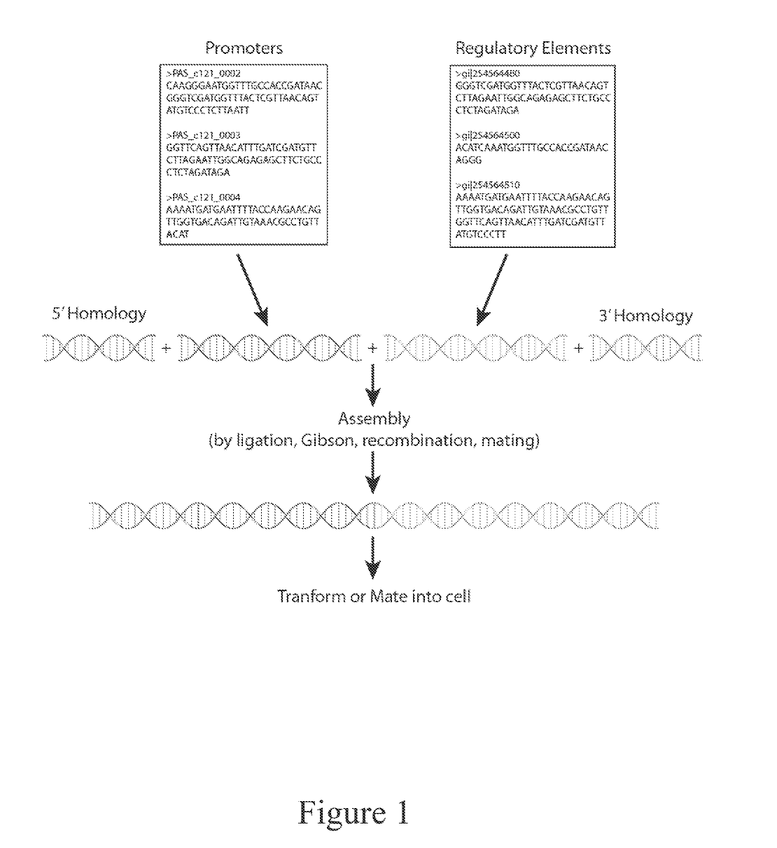

After identification of promoters and regulatory sequences in the selected cell strain, as well as any potential heterologous promoters or regulatory sequences, a library of all promoter-regulatory element pairs is created. The goal of this step is to design and create a library consisting of physical DNA sequences in which (in a preferred embodiment) every selected promoter element is paired with every selected regulatory element. Alternatively, as described below, the library can contain a set or a subset of selected promoter elements paired with a set or a subset of selected regulatory elements. FIG. 1 shows an example of selecting promoter-regulatory element pairs and assembling them into vectors.

Alternatively, if this approach results in too many elements to effectively screen, a subset of promoters and regulatory elements may be used to create the library. This subset can be randomly selected or can be chosen based on the best available understanding of the organism and product production pathway. For example, in P. pastoris, the typically used protein production pathway uses methanol as an inducing agent. Therefore, the library size can be reduced by limiting promoters to those that are activated by the cell during the methanol-consuming phase of its metabolism. These promoters can be identified from literature or using microarrays, RNA transcriptome sequencing, or other methods to determine which genes are activated by methanol. In this case the promoters for genes activated by methanol are selected for the library.

In another embodiment, each element of the library may be synthesized. In still another embodiment, each element of the library may be acquired directly from the organism's genome by synthesizing a pair of oligonucleotide primers for each element, and performing a PCR reaction using the organism's genomic DNA as the template. This operation can be performed in parallel for each library element using multi-well plates, and may be automated using robotics.

B. Library Construction

In addition to promoters and regulatory elements, each library member includes additional DNA elements required to insert the member into the target cell and make it functional. This generally takes the form of either a vector backbone containing a replication origin and a selection marker (typically antibiotic resistance, although many other methods are possible), or a linear fragment that enables incorporation into the target cell's chromosome. The elements should correspond to the organism and insertion method chosen.

Once the library elements are selected, construction of the library can be performed in many different ways. In an embodiment, a DNA synthesis service or a method to individually make every library element may be used. Future synthesis technologies may make this approach more feasible with larger libraries.

Once the DNA for each element of the library (including the additional elements required for insertion and operation) is acquired, the elements must be assembled (FIG. 1). There are many possible assembly methods including (but not limited to) restriction enzyme cloning, blunt-end ligation, and overlap assembly [see, e.g., Gibson, D. G., et al., Enzymatic assembly of DNA molecules up to several hundred kilobases. Nature methods, 6(5), 343-345 (2009), and GeneArt Kit (http://tools.invitrogen.com/content/sfs/manuals/geneart_seamless_cloning_and_assembly_man.pdf)]. Overlap assembly provides a method to ensure all of the elements get assembled in the correct position and does not introduce any undesired sequences into library elements. In one preferred embodiment, the assembly method allows for a “one-pot” assembly, in which all elements of the library are combined into a single mixture and the reaction is performed generating all possible combinations of library members. In an embodiment of the “one-pot” assembly, restriction enzymes and blunt-end assembly are used to form the elements of the library. A universally identical region between the promoter and the regulatory element can be used to enable overlap assembly for the “one-pot” assembly method. In a preferred embodiment, this universally identical region comprises a ribosome binding site (bacteria) or kozak sequence (yeast) or similar element. The method described above results in a solution containing assembled DNA with the full coverage of the library elements in an expression cassette (in e.g., a vector or linear fragment) suitable for incorporation into a cell.

C. Introducing Library into Target Cell Population

The library generated above is inserted into target cells using standard molecular biology techniques, e.g., molecular cloning. In an embodiment, the target cells are already engineered or selected such that they already contain the genes required to make the desired product, although this may also be done during or after library insertion.

Depending on the organism and library element type (plasmid or genomic insertion), several known methods of inserting the library DNA into the cells may be used. These may include, for example, transformation of microorganisms able to take up and replicate DNA from the local environment, transfection of mammalian cell culture, transformation by electroporation or chemical means, transduction with a virus or phage, mating of two or more cells, or conjugation from a different cell.

Several methods are known in the art to introduce recombinant DNA in bacterial cells that include but are not limited to transformation, transduction, and electroporation, see Sambrook, et al., Molecular Cloning: A Laboratory Manual (1989), Second Edition, Cold Spring Harbor Press, Plainview, N.Y. Non-limiting examples of commercial kits and bacterial host cells for transformation include NovaBlue Singles™ (EMD Chemicals Inc, NJ, USA), Max Efficiency® DH5α™, One Shot® BL21 (DE3) E. coli cells, One Shot® BL21 (DE3) pLys E. coli cells (Invitrogen Corp., Carlsbad, Calif., USA), XL1-Blue competent cells (Stratagene, Calif., USA). Non limiting examples of commercial kits and bacterial host cells for electroporation include Zappers™ electrocompetent cells (EMD Chemicals Inc, NJ, USA), XL1-Blue Electroporation-competent cells (Stratagene, Calif., USA), ElectroMAX™ A. tumefaciens LBA4404 Cells (Invitrogen Corp., Carlsbad, Calif., USA).

Several methods are known in the art to introduce recombinant nucleic acid in eukaryotic cells. Exemplary methods include transfection, electroporation, liposome mediated delivery of nucleic acid, microinjection into to the host cell, see Sambrook, et al., Molecular Cloning: A Laboratory Manual (1989), Second Edition, Cold Spring Harbor Press, Plainview, N.Y. Non-limiting examples of commercial kits and reagents for transfection of recombinant nucleic acid to eukaryotic cell include Lipofectamine™ 2000, Optifect™ Reagent, Calcium Phosphate Transfection Kit (Invitrogen Corp., Carlsbad, Calif., USA), GeneJammer® Transfection Reagent, LipoTAXI® Transfection Reagent (Stratagene, Calif., USA). Alternatively, recombinant nucleic acid may be introduced into insect cells (e.g. sf9, sf21, High Five™) by using baculo viral vectors.

The library DNA is inserted so that cells in the culture each contain a single library element. In an embodiment, this is accomplished by using a larger number of cells compared with the number of library elements. In another embodiment, the number of cells is several times larger than the number of library elements.

Cells containing a library element are cultured and clones containing unique library elements are isolated. The cells containing the library elements are isolated so that each clone (a strain of the cell type with a single library element) can be tested separately. In an embodiment, this is done by spreading the culture on one or more plates of culture media containing a selective agent (or lack of one) that will ensure that only cells containing a library element survive and reproduce. This specific agent may be an antibiotic (if the library contains an antibiotic resistance marker), a missing metabolite (for auxotroph complementation), or other means of selection. The cells are grown into individual colonies, each of which contains a single clone of the library.

Colonies are screened for desired production of a protein, metabolite, or other product. In an embodiment, screening identifies recombinant cells having the highest (or high enough) product production titer or efficiency. This screening can be performed many ways, depending on the product. In one aspect, culture plate selection on a medium comprising a selective agent (or lack of one) is a sufficient screen. For example if the product conveys a resistance to a toxin, plates can be made with increasing quantity of toxin so that only cells with high product production titer survive and reproduce.

In another embodiment, colonies can be picked (manually or robotically) into multi-well culture plates and grown in liquid culture under conditions similar to those selected for use during eventual product synthesis with the selected recombinant clonal colony. This approach allows the screen to select not only for production of a desired product, but also for product secretion, if desired, since the assay can be designed to look at culture supernatants and cell contents separately.

Several other types of screening assays are well-known in the art. In one aspect, the protein product is grown in Pichia pastoris under the control of a methanol-inducible promoter (i.e., AOX1 or AOX2) and the protein is tagged with a fluorescent, epitope, enzymatic, or luminescent marker. The protein product can also be grown under the control of a constitutive promoter (i.e., GAP or GCW14). This assay can be performed by growing individual clones, one per well, in multi-well culture plates. Once the cells have reached an appropriate biomass density, they are induced with methanol. After a period of time, typically 24-72 hours of induction, the cultures are harvested by spinning in a centrifuge to pellet the cells and removing the supernatant. The supernatant from each culture can then be viewed in a fluorescence reader. In this embodiment of the assay, the best producing and secreting strains show greater fluorescence. In a further embodiment, this process is at least partially automated with robotics in order to screen a large number of clones in a relatively short amount of time and minimal effort.

Once the clones with sufficient product production are identified, those cultures may be located, either as colonies on their selective plate, as assay cultures, or as duplicate master stocks as described in step 7. These can be grown and used for production directly, or their DNA can be sequenced in order to specifically identify the library element that they contain. Once identified this element can be re-constructed for specific testing and verification of the activity. This information can then be used to create new production strains or to help design additional improvements.

### III. ISOLATION OF GENETIC IMPROVEMENTS

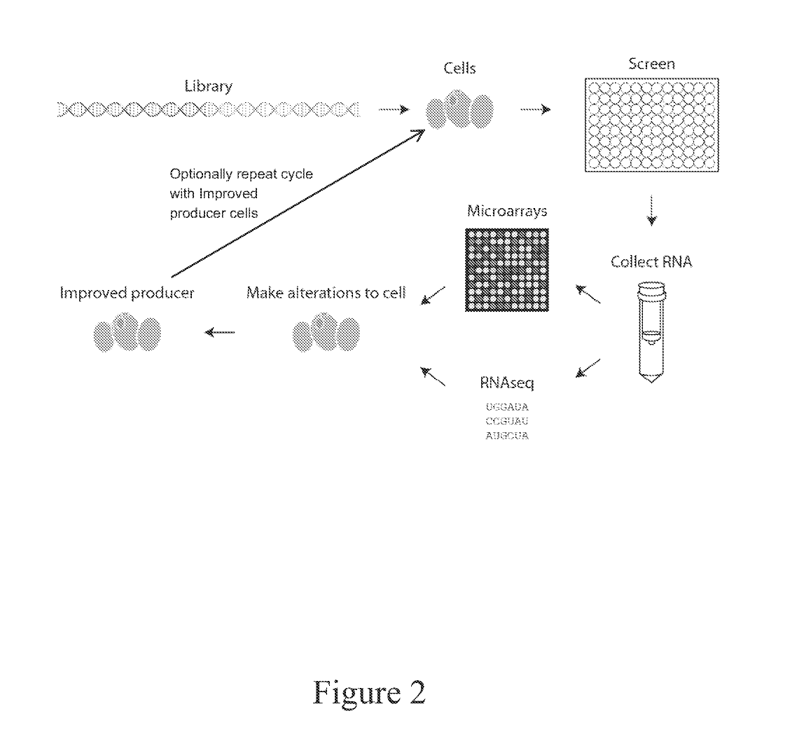

Cells showing improved product production are identified. To better understand the induced cellular changes, an embodiment of the method employs analysis to determine which genes or RNA-based regulators are affected. This method identifies those improvements and implements them individually. This method can be implemented on any cell in which targeted alterations to the identified genes or RNA-based regulators are effective to improve product production. Steps of an embodiment of a method for isolating genetic improvements and engineering a host cell is shown in FIG. 2.

A natural or engineered cell capable of producing the desired product is selected. A cell can be selected from, but not limited to, one of the following: a prokaryotic cell, Escherichia coli, Bacillus subtilis, a eukaryotic cell, Pichia pastoris, Hansenula polymorphs, and Saccharomyces cerevisiae. The cell can include enhancements to allow for specific (potentially heterologous) product production. For example, a P. pastoris cell might have a gene encoding spider silk protein incorporated into the genome to express spider silk protein product.

A promoter—regulatory element library approach is generated (e.g., as described above). A cell producing a protein or metabolite of interest is transformed or mated with a library of promoter—regulatory elements. These elements are encoded in DNA with a promoter operably linked to a regulatory element. In a preferred embodiment, the promoter is 5′ to the regulatory element. Regulatory elements include but are not limited to regulatory proteins (e.g., transcription factors), chaperones, signaling proteins, RNAi molecules, antisense RNA molecules, microRNAs and RNA aptamers. The library is screened as previously described and improved producers are isolated. Isolated cells with desired production of the target molecule are identified and isolated.

When the cell with the desired target molecule production profile (i.e., “the improved cell”) is identified and isolated, it is tested to identify the altered metabolic state of the cell. In an embodiment, the cell is grown in product producing conditions and total RNA is harvested. The specific harvest can be done in a number of ways, including commercial kit (RNeasy from Qiagen for example) or in house protocols such as phenol-chloroform extraction. This measurement of total RNA provides one method to identify the altered metabolic state of the improved cell. In an embodiment, a reference control, e.g., the cell selected prior to library transformation may be used as a baseline for measurement of the metabolic state of the cell. This cell is grown in product producing conditions identical to the cell identified to have the desired product producing properties and total RNA harvested from the control cell.

In an embodiment, transcripts of interest can be selected for using, e.g., rRNA depletion or mRNA purification. The total RNA isolated in the measurement of total RNA contains only a small fraction of messenger RNA (mRNA), which indicates transcription level of genes, and non-coding RNA (ncRNA), which indicates the presence of regulatory RNAs. The majority of RNA in the cell is ribosomal RNA (rRNA) and transfer RNA (tRNA). In an embodiment, mRNA or ncRNA is enriched using a commercial kit for ribosomal RNA depletion (e.g., Ribo-Zero from Epicentre). Alternatively if only mRNA is desired, a poly-T purification will isolate message transcripts and is available in commercial kit format (e.g., DynaBeads from Invitrogen).

In an embodiment, enriched RNA from the optimized cell is used to identify and quantify transcripts in the improved cell that are altered in presence and magnitude of expression from the control cell. The difference between the improved and control cells is measured, e.g., by RNA sequencing (RNAseq) of the transcriptome or microarray analysis. In RNAseq the whole sample is prepared for next gen sequencing (e.g., Illumina GXII platform) using the appropriate RNA sequencing kit. In an embodiment, the amount of sequence generated is tuned to give greater than or equal to 20 times coverage of the available transcripts and give quantitative data on the level of expression. In an embodiment, microarray analysis is performed on a chip arrayed with a series of small sequences (e.g., probes) for RNA transcripts in the cell. A commercial provider such as Affymetrix commonly produces and supplies such microarrays. The RNA transcripts are allowed to anneal to the microarray surface, washed to remove non-specifically annealed transcripts, and then analyzed using fluorescent dye to determine the identity and magnitude of expression for each target.

The results of the profile from the improved cell and control cell are compared to find specific differences in expression. These could include, but are not limited to, reduced or enhanced expression of protein coding genes, ncRNAs, and other RNA species. These changes in identity of expressed transcripts and the expression level are noted for making specific modifications.

The identified changes in transcription level between the improved cell and control cell are implemented in a host cell similar to or identical to the control cell. In an embodiment, these identified changes are provided by a third party. In another embodiment, alterations for the cell are identified by the methods as described herein, and directly incorporated into a host cell. These changes can include but are not limited to removing DNA from the cell's genome which encodes genes or ncRNA regions, adding extra copies of DNA to the cells genome for genes and ncRNAs, altering the expression level of specific genes and ncRNAs by changing the promoter in driving transcription. In an embodiment, each change is made to the cell without the use of the promoter-regulatory element pair identified from the library screening.

In an embodiment, the steps outlined above can be repeated as a cycle to continuously improve the selected cell towards a desired production of a compound.

As is well known in the art, enzyme activities can be measured in various ways. For example, the pyrophosphorolysis of OMP may be followed spectroscopically (Grubmeyer et al., (1993) J. Biol. Chem. 268:20299-20304). Alternatively, the activity of the enzyme can be followed using chromatographic techniques, such as by high performance liquid chromatography (Chung and Sloan, (1986) J. Chromatogr. 371:71-81). As another alternative the activity can be indirectly measured by determining the levels of product made from the enzyme activity. These levels can be measured with techniques including aqueous chloroform/methanol extraction as known and described in the art (Cf M. Kates (1986) Techniques of Lipidology; Isolation, analysis and identification of Lipids. Elsevier Science Publishers, New York (ISBN: 0444807322)). More modern techniques include using gas chromatography linked to mass spectrometry (Niessen, W. M. A. (2001). Current practice of gas chromatography—mass spectrometry. New York, N.Y: Marcel Dekker. (ISBN: 0824704738)). Additional modern techniques for identification of recombinant protein activity and products including liquid chromatography-mass spectrometry (LCMS), high performance liquid chromatography (HPLC), capillary electrophoresis, Matrix-Assisted Laser Desorption Ionization time of flight-mass spectrometry (MALDI-TOF MS), nuclear magnetic resonance (NMR), near-infrared (NIR) spectroscopy, viscometry (Knothe, G (1997) Am. Chem. Soc. Symp. Series, 666: 172-208), titration for determining free fatty acids (Komers (1997) Fett/Lipid, 99(2): 52-54), enzymatic methods (Bailer (1991) Fresenius J. Anal. Chem. 340(3): 186), physical property-based methods, wet chemical methods, etc. can be used to analyze the levels and the identity of the product produced by the organisms of the present invention. Other methods and techniques may also be suitable for the measurement of enzyme activity, as would be known by one of skill in the art.

The following examples are for illustrative purposes and are not intended to limit the scope of the present invention.

### Example 1

### Method for Improving Metabolite or Small Molecule Production

A cell capable of producing a desired protein, macromolecule or metabolite (i.e., products) is transformed or mated to introduce a library of DNA elements with one or more pairs of genetic promoters and genes encoding regulatory elements (e.g., transcription factors or other signaling proteins). The resulting cells are isolated on selective media plates (by auxotrophy or antibiotic resistance marker) and individual clones are isolated for further testing. Individual clones are tested by selective plate based assay or liquid culture assay under product producing conditions. The cells are analyzed for production of products in the culture broth and/or inside the cell and products may require purification. A metabolite product is detected and quantified by any combination of enzymatic assay, liquid chromatography, mass spectrometry, gas chromatography, colorimetric assay, electrophoretic mobility assay, nuclear magnetic resonance. Based upon library size and screening capacity a number of clones are screened for product formation and the best producers are retested and subjected to additional rounds of improvement by introduction of a library of promoter-signaling factor DNA.

The process described above can be performed using RNA as regulatory elements other than signaling proteins. A promoter-small RNA fusion into a cell capable of producing a desired protein, macromolecule or metabolite. This is followed by isolating cells and testing for desired cell properties, e.g., production of desired products. Alternatively a library of promoter-small RNA fusions is introduced into a population of cells capable of producing a desired protein, macromolecule or metabolite. A random 10 mer RNA regulatory element would lead to ˜1 million (4¹⁰) members in a regulatory RNA element library.

### Example 2

### Generating a Library of Promoters and Regulatory Elements for Pichia Pastoris

We describe here a method for performing whole cell evolution by fusing random Pichia promoters to random Pichia nucleotide binding proteins (e.g., transcription factors) to achieve changes in cellular regulation and metabolism. These changes modify silk production and secretion.

The recent sequencing of Pichia pastoris identified 5,313 protein coding genes. Work with pfam and other prediction tools allowed us to identify ˜350 putative transcriptions after removing DNA polymerases, telomerases, helicases, and other obvious non-transcription factor proteins as described below. Pichia promoters (up to a few kilobases upstream of each open reading frame) are isolated from a subset or the entirety of protein coding regions in the genome. Using these two sets of parts we create ˜1.8M single combinations to create new regulatory dynamics that perturb the cell.

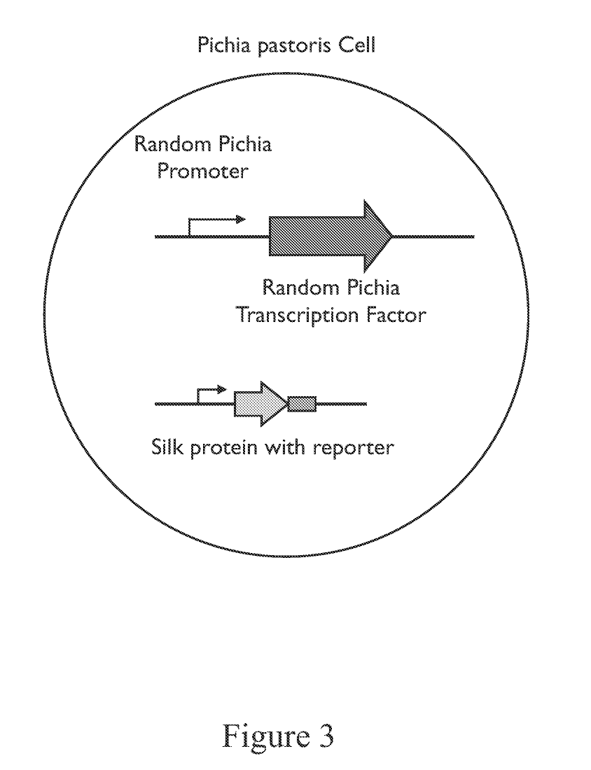

A Pichia strain is transformed with a silk protein gene (e.g., major ampullate silk protein 1 (MaSp1)) construct operably linked to a pAOX1 promoter and a chosen library of promoter-TF pairs (FIG. 3). To generate a library of regulatory elements for Pichia pastoris, the UniProt database was searched for characterized and putative regulatory elements from the GS115 (NRRL Y15851) strain. The pAOX1 promoter is encoded by the following nucleotide sequence (GenBank Accession No: JQ519688.1) (SEQ ID NO: 235):

AACATCCAAAGACGAAAGGTTGAATGAAACCTTTTTGCCATCCGACATCC
ACAGGTCCATTCTCACACATAAGTGCCAAACGCAACAGGAGGGGATACAC
TAGCAGCAGACCGTTGCAAACGCAGGACCTCCACTCCTCTTCTCCTCAAC
ACCCACTTTTGCCATCGAAAAACCAGCCCAGTTATTGGGCTTGATTGGAG
CTCGCTCATTCCAATTCCTTCTATTAGGCTACTAACACCATGACTTTATT
AGCCTGTCTATCCTGGCCCCCCTGGCGAGGTTCATGTTTGTTTATTTCCG
AATGCAACAAGCTCCGCATTACACCCGAACATCACTCCAGATGAGGGCTT
TCTGAGTGTGGGGTCAAATAGTTTCATGTTCCCCAAATGGCCCAAAACTG
ACAGTTTAAACGCTGTCTTGGAACCTAATATGACAAAAGCGTGATCTCAT
CCAAGATGAACTAAGTTTGGTTCGTTGAAATGCTAACGGCCAGTTGGTCA
AAAAGAAACTTCCAAAAGTCGGCATACCGTTTGTCTTGTTTGGTATTGAT
TGACGAATGCTCAAAAATAATCTCATTAATGCTTAGCGCAGTCTCTCTAT
CGCTTCTGAACCCCGGTGCACCTGTGCCGAAACGCAAATGGGGAAACACC
CGCTTTTTGGATGATTATGCATTGTCTCCACATTGTATGCTTCCAAGATT
CTGGTGGGAATACTGCTGATAGCCTAACGTTCATGATCAAAATTTAACTG
TTCTAACCCCTACTTGACAGCAATATATAAACAGAAGGAAGCTGCCCTGT
CTTAAACCTTTTTTTTTATCATCATTATTAGCTTACTTTCATAATTGCGA
CTGGTTCCAATTGACAAGCTTTTGATTTTAACGACTTTTAACGACAACTT
GAGAAGATCAAAAAACAACTAATTATTGAAA

Specifically, the UniProt database was searched for nucleotide binding proteins, as these are the likely effectors of network regulation (such as transcription factors). The following keywords were excluded from the results, because these proteins are likely regulators of cell maintenance and growth, not protein production, secretion, or folding: polymerase, histone, ligase, topoisomerase, endonuclease, helicase, DNA mismatch repair mutS family, DNA mismatch repair, DNA repair, exonuclease, telomerase, and RNase. Certain of these keywords, e.g., RNase, were excluded to reduce library, size, although they may be included as modulating RNase regulation could easily affect mRNA or tRNA levels.

Furthermore, because one anticipates they affect protein expression, secretion, stability, and solubility, regulatory elements characterized in the academic literature to be involved in protein folding (chaperones), the unfolded protein response, and the methanol utilization pathway were included. For example, these proteins include BFR2, BMH1, COG6, FLD1, and DAS2.

Putative functional characterizations were performed by the InterPro database, which automatically classifies proteins based on sequence features. This search resulted in 354 putative nucleotide binding proteins or other regulatory elements, as electronically inferred by InterPro within the UniProt database. The resulting RefSeq sequences linked to the 354 putative regulatory elements are listed in Table 1.

Primers were generated for each sequence by identifying the forward and reverse primers that had a melting temperature greater than or equal to 60° C., and were between 15 to 30 bases in length. Maximum length was prioritized over melting temperature (e.g., certain primers certain had a melting temperature <60° C., but were 30 bases long).

Melting temperature was calculated based on modified Breslauer thermodynamics, as described in: W. Rychlik, W. J. Spencer and R. E. Rhoads, “Optimization of the annealing temperature for DNA amplification in vitro”, Nucleic Acids Research, Vol. 18, No. 21 6409.

A promoter library is generated for Pichia pastoris by obtaining 1500 bases upstream of every open reading frame (i.e., ORF). For a eukaryote, 1500 bases are sufficient to likely capture the promoter sequence. In addition, known and characterized promoter sequences are added, such as AOX 1 and AOX2. These promoters are induced under methanol, and are of different strengths, which will lead to inducible network rewiring of different magnitudes.

From the transformation individual clones are picked into 2.4 mL 96 well plates, grown, induced on methanol and screened for protein expression and secretion. This results in emergent network behavior providing us with a large search space of cellular rewiring—leading to new phenotypes with altered carbon flux, varied stress tolerances, etc. Similar results, using different methodologies, have been seen in Saccharomyces cerevisiae (Alper, Hal, et al., Science 314, 1546 (2006)). The resulting colonies are isolated to screen for improved expression, secretion, and processing of silk protein. The silk protein can be native to the host cell. Alternatively, the silk protein can be recombinantly fused to a detection marker (e.g., an epitope tag, fluorescent protein, firefly luciferase, or beta galactosidase). A variety of network effects, e.g., downregulation of protein degradation, or upregulation of vesicular trafficking, can result in the measured phenotype (e.g., increased silk protein production) of the recombinant host cells. A subset of the recombinant cells with a selected phenotype can be re-tested and/or subjected to additional rounds of library construction, transformation and testing, as described above.

TABLE 1
RefSeq ID's of putative regulatory elements extracted
from the UniProt database on Mar. 12, 2012.

| | | |
|---|---|---|
| XM_002492229.1. | XM_002492119.1. | XM_002494112.1. |
| XM_002493036.1. | XM_002491990.1. | XM_002492960.1. |
| XM_002490860.1. | XM_002492738.1. | XM_002490386.1. |
| XM_002489482.1. | XM_002493585.1. | XM_002491378.1. |
| XM_002491060.1. | XM_002490991.1. | XM_002494295.1. |
| XM_002493188.1. | XM_002492620.1. | XM_002492667.1. |
| XM_002493877.1. | XM_002491183.1. | XM_002493701.1. |
| XM_002491971.1. | XM_002491374.1. | XM_002491091.1. |
| XM_002489647.1. | XM_002492310.1. | XM_002491375.1. |
| XM_002493398.1. | XM_002489393.1. | XM_002493562.1. |
| XM_002491645.1. | XM_002489363.1. | XM_002490353.1. |
| XM_002490965.1. | XM_002489575.1. | XM_002491403.1. |
| XM_002490082.1. | XM_002490253.1. | XM_002491779.1. |
| XM_002489334.1. | XM_002492781.1. | XM_002491735.1. |
| XM_002490805.1. | XM_002493118.1. | XM_002489650.1. |
| XM_002493563.1. | XM_002490469.1. | XM_002492681.1. |
| XM_002492851.1. | XM_002492513.1. | XM_002492279.1. |
| XM_002494060.1. | XM_002493098.1. | XM_002489974.1. |
| XM_002492590.1. | XM_002491307.1. | XM_002494028.1. |
| XM_002493717.1. | XM_002493851.1. | XM_002491802.1. |
| XM_002492008.1. | XM_002493393.1. | XM_002492074.1. |
| XM_002490439.1. | XM_002491733.1. | XM_002492884.1. |
| XM_002492430.1. | XM_002493377.1. | XM_002493832.1. |
| XM_002492684.1. | XM_002493290.1. | XM_002490452.1. |
| XM_002490339.1. | XM_002491552.1. | XM_002492234.1. |
| XM_002493553.1. | XM_002489306.1. | XM_002492110.1. |
| XM_002492580.1. | XM_002489400.1. | XM_002493538.1. |
| XM_002493914.1. | XM_002492191.1. | XM_002494020.1. |
| XM_002489990.1. | XM_002492375.1. | XM_002491409.1. |
| XM_002490608.1. | XM_002490688.1. | XM_002490325.1. |
| XM_002492126.1. | XM_002492572.1. | XM_002491761.1. |
| XM_002491260.1. | XM_002494138.1. | XM_002492805.1. |
| XM_002491454.1. | XM_002492458.1. | XM_002493565.1. |
| XM_002491778.1. | XM_002489481.1. | XM_002492726.1. |
| XM_002490205.1. | XM_002491299.1. | XM_002492621.1. |
| XM_002490399.1. | XM_002489355.1. | XM_002492236.1. |
| XM_002492931.1. | XM_002490934.1. | XM_002491250.1. |
| XM_002489537.1. | XM_002490282.1. | XM_002489552.1. |
| XM_002489451.1. | XM_002489395.1. | XM_002490198.1. |
| XM_002490861.1. | XM_002489633.1. | XM_002489422.1. |
| XM_002489326.1. | XM_002493084.1. | XM_002492659.1. |
| XM_002489607.1. | XM_002489316.1. | XM_002491941.1. |
| XM_002492601.1. | XM_002490926.1. | XM_002491226.1. |
| XM_002493024.1. | XM_002490606.1. | XM_002491873.1. |
| XM_002492403.1. | XM_002490284.1. | XM_002490851.1. |
| XM_002491084.1. | XM_002492825.1. | XM_002491763.1. |
| XM_002491306.1. | XM_002490293.1. | XM_002490234.1. |
| XM_002490618.1. | XM_002492982.1. | XM_002490433.1. |
| XM_002491952.1. | XM_002489339.1. | XM_002493995.1. |
| XM_002493699.1. | XM_002493176.1. | XM_002490819.1. |
| XM_002491672.1. | XM_002493454.1. | XM_002490876.1. |
| XM_002490582.1. | XM_002490168.1. | XM_002492496.1. |
| XM_002490065.1. | XM_002489464.1. | XM_002493456.1. |
| XM_002493710.1. | XM_002492012.1. | XM_002490359.1. |
| XM_002493639.1. | XM_002491617.1. | XM_002490613.1. |
| XM_002491220.1. | XM_002493703.1. | XM_002491793.1. |
| XM_002490432.1. | XM_002490047.1. | XM_002492470.1. |
| XM_002489571.1. | XM_002490753.1. | XM_002493768.1. |
| XM_002494123.1. | XM_002491677.1. | XM_002493526.1. |
| XM_002492713.1. | XM_002493462.1. | XM_002492431.1. |
| XM_002492425.1. | XM_002489423.1. | XM_002493528.1. |
| XM_002493323.1. | XM_002493265.1. | XM_002492957.1. |
| XM_002492744.1. | XM_002492977.1. | XM_002490647.1. |
| XM_002490212.1. | XM_002494169.1. | XM_002493464.1. |
| XM_002489766.1. | XM_002492342.1. | XM_002490249.1. |
| XM_002490096.1. | XM_002490903.1. | XM_002493578.1. |
| XM_002489329.1. | XM_002492298.1. | XM_002491012.1. |
| XM_002489824.1. | XM_002489417.1. | XM_002492176.1. |
| XM_002490029.1. | XM_002493250.1. | XM_002493545.1. |
| XM_002489583.1. | XM_002492027.1. | XM_002490055.1. |
| XM_002489653.1. | XM_002490112.1. | XM_002491938.1. |
| XM_002491585.1. | XM_002492746.1. | XM_002491711.1. |
| XM_002490355.1. | XM_002493501.1. | XM_002491668.1. |
| XM_002491099.1. | XM_002489794.1. | XM_002491607.1. |
| XM_002493588.1. | XM_002493119.1. | XM_002489957.1. |
| XM_002491699.1. | XM_002490905.1. | XM_002493643.1. |
| XM_002490476.1. | XM_002489994.1. | XM_002492144.1. |
| XM_002489917.1. | XM_002489678.1. | XM_002491078.1. |
| XM_002490679.1. | XM_002491494.1. | XM_002493324.1. |
| XM_002490574.1. | XM_002489321.1. | XM_002492349.1. |
| XM_002491123.1. | XM_002494212.1. | XM_002493819.1. |
| XM_002492056.1. | XM_002491867.1. | XM_002492996.1. |
| XM_002490417.1. | XM_002490629.1. | XM_002489525.1. |
| XM_002490682.1. | XM_002494225.1. | XM_002490199.1. |
| XM_002489855.1. | XM_002489944.1. | XM_002493610.1. |
| XM_002491856.1. | XM_002491967.1. | XM_002492913.1. |
| XM_002491365.1. | XM_002493142.1. | XM_002489754.1. |
| XM_002492907.1. | XM_002489425.1. | XM_002491912.1. |
| XM_002492026.1. | XM_002493115.1. | XM_002492406.1. |
| XM_002489397.1. | XM_002489468.1. | XM_002493166.1. |
| XM_002493705.1. | XM_002491859.1. | XM_002489382.1. |
| XM_002492772.1. | XM_002492244.1. | XM_002492657.1. |
| XM_002489411.1. | XM_002493392.1. | XM_002489808.1. |
| XM_002491030.1. | XM_002490329.1. | XM_002492717.1. |
| XM_002490495.1. | XM_002489783.1. | XM_002493170.1. |
| XM_002493757.1. | XM_002494257.1. | XM_002490833.1. |
| XM_002492261.1. | XM_002492077.1. | XM_002492566.1. |
| XM_002490710.1. | XM_002491023.1. | XM_002491527.1. |
| XM_002490735.1. | XM_002490648.1. | XM_002490414.1. |
| XM_002491841.1. | XM_002492946.1. | XM_002494290.1. |
| XM_002491305.1. | XM_002489784.1. | XM_002490105.1. |
| XM_002492113.1. | XM_002490795.1. | XM_002491369.1. |
| XM_002494282.1. | XM_002493268.1. | XM_002490507.1. |
| XM_002489364.1. | XM_002491910.1. | XM_002494117.1. |
| XM_002492386.1. | XM_002493281.1. | XM_002489659.1. |
| XM_002493244.1. | XM_002489408.1. | XM_002489841.1. |
| XM_002494199.1. | XM_002492844.1. | XM_002489995.1. |
| XM_002490614.1. | XM_002491232.1. | XM_002491017.1. |
| XM_002493834.1. | XM_002491270.1. | XM_002491909.1. |
| XM_002491676.1. | XM_002493138.1. | XM_002494255.1. |
| XM_002492692.1. | XM_002493806.1. | XM_002490283.1. |
| XM_002494115.1. | XM_002494219.1. | XM_002489658.1. |
| XM_002494042.1. | XM_002491081.1. | XM_002493318.1. |
| XM_002491626.1. | XM_002493050.1. | XM_002489950.1. |
| XM_002490580.1. | XM_002493238.1. | XM_002490770.1. |
| XM_002492703.1. | XM_002490766.1. | XM_002494143.1. |
| XM_002491892.1. | XM_002491888.1. | XM_002491312.1. |
| XM_002489654.1. | XM_002494285.1. | |

### Example 3

### Robotic Setup for High-Throughput Screening of Host Cells

A setup designed for high-throughput screening of secreted protein production in yeast is described herein. This setup consists of five main parts: colony picker, incubating shaker, centrifuge, liquid handling robot and a scanner/detector.

The colony picker is used to select individual clones (colonies) from the agar media plates and place each into a separate well of a multi-well culture plate. We use a Genetix QPix for this purpose

The incubating shaker is capable of a high density for deepwell culture plates and be able to control for optimal temperatures, shaking rates and humidity to achieve conditions similar to those that will be used for production. In a preferred embodiment, for Pichia pastoris, the optimal conditions are achieved in 96-well deep culture plates (2.4 mL total volume), at temperatures between 15° C. and 30° C., and at shaking rates up to 1000 rpm with a 3 mm throw. In an embodiment, an InforsHT Microtron capable of growing up to 60 plates (5760 wells) at once is used.

The centrifuge is able to pellet cells in the plates (typically at least 3000×g force is required). Since this machine is typically the bottleneck in the system and higher capacity centrifuges are not readily available, multiple centrifuges may be required.

The liquid handling robot is used to feed the cultures, harvest the completed cultures, and perform assays. Regular additions of a carbon source provide optimal growth and regular additions of inducing agent (methanol in Pichia) are optimal. A dual arm Beckmann BioMek FX is used for this purpose.

The scanner/detector is used to read plate-based solutions and detect protein concentrations. Several assays can be performed depending on the protein and media composition. Fluorescence, luminance, absorbance, or another method of detection can be used. Preferably, the detector will be directly connected to the robot to minimize the amount of human interaction required. A Molecular Devices SpectraMax M2 is used to measure absorbance and fluorescence.

The process comprises the following steps: 1. Fill 60 96-deepwell plates with culture media using liquid handler. 2. 5760 colonies (including controls) are picked into the plate wells using a colony picker. 3. The plates are placed into the incubating shaker and grown under the appropriate conditions. 4. Periodically, the plates are taken out of the incubator and placed on the liquid handler, where additional feed is added and culture density measurements are made using the attached scanner. The plates are then put back into the incubator. 5. Once the cultures reach the correct density (typically ˜24-48 hours for Pichia), they are induced by pelleting the cells in the centrifuge, decanting the media, and again placing them on the liquid handler, which will add the appropriate amount of induction media (media with methanol as a sole carbon source for Pichia) and the plates again placed back in the incubator. 6. Periodically additional inducer is added to counteract evaporation and consumption by the cells. Again, this is done with the liquid handler. 7. Once a sufficient amount of induction time has elapsed (for Pichia, typically 12-72 hours), the plates are removed from the incubator and spun on the centrifuge(s). 8. The now clarified culture media is removed from each plate and placed into a separate multi-well assay plate using the liquid handler. The liquid handler then adds any necessary reagents for the assay to occur. For example, a beta-galactosidase assay requires the compound ortho-nitrophenyl-galactose (ONPG) to be added. Alternatively, a fluorescently tagged protein does not require any additional reagent. 9. The liquid handler then places the assay plates into the scanner where the results of the process are read.

### Example 4

### Plate Uniformity Testing

When extending laboratory protocols for use in 96-well plates or other high-throughput platforms, the multiple transfers of small volumes can often lead to accumulation of significant natural variation between ostensibly identical samples. To be able to accurately detect high producers of the desired protein or compounds, it is therefore important to reliably quantify the amount of reliable uniformity in all steps of a given protocol. This example addresses this issue.

Cell cultures are grown in many small volumes (<1 ml per culture) and high densities (96 experiments/plates, multiple plates), induced to express and secrete the desired proteins, and sampled in parallel to assess the amount of protein produced in each individual culture.

To quantify the reliability of the results of these assays, we have assessed the variability introduced by each of the liquid transfer steps. The primary two steps include:
1) Removal of turbid cell culture from 96-well or other high-throughput plates to assess cell optical density in parallel.
2) Removal of culture supernatant after pelleting cells, to calculate extracellular protein density with a variety of metrics (fluorescence or luminescence; or Bradford, BCA, and other standard protein concentration assays)

In both cases, precise removal of liquid from each culture volume is crucial for assay uniformity. To this end, we quantified the reliability of liquid transfer steps for fluorescence, cell density, and BCA (bicinchoninic)/Bradford plate assays.

Noise in the assays also accrues due to factors including oxygenation levels of different plates or wells in incubator shakers, natural variability in cell cultures. Steps for testing plate uniformity comprise:
1) Comparing the accuracy of manual pipetting against that of a recently acquired liquid handling robot.
2) Comparing the variation between calculation of identical initial protein concentrations according to BCA and Bradford protein assay kits.
3) Using a fluorescent protein construct to assess the variation in protein expression levels between adjacent wells in a 96-well plate when started from identical cell cultures.
4) Normalizing the above plate's data according to the cell density within each well, to determine if the most saturated cell densities yield lower levels of soluble, secreted fluorescent protein.
5) Testing cell growth rate and saturation point in 96-well plates with different well depths and stacking conditions, to determine how much the growth of many plates in a shaker will affect plate-to-plate uniformity.
   Test 1: Accuracy of Manual Vs. Robotic Liquid Transfer Volumes

Turbid cells at an initial optical density of 6.5 at 600 nm were diluted tenfold into phosphate-buffered saline (PBS) at pH 7.4, into final volumes of 250 μl per well, in clear Costar 96-well optical plates. This transfer was done manually in one plate and using a Biomek FX liquid handler in another. All samples were mixed by pipetting up and down three times to ensure consistent turbidity within each well. We measured optical density data using a Spectramax 250 plate reader at 600 nm wavelength, and corrected values by the average background signal of 250 μl of PBS (0.038, in these plates). Heatmaps of the measured fractional variation around the mean optical density of each plate were obtained. We calculated fractional variation by dividing each individual well's optical density by the mean optical density of all 96 wells per plate, then subtracting 1 from all resulting numbers.

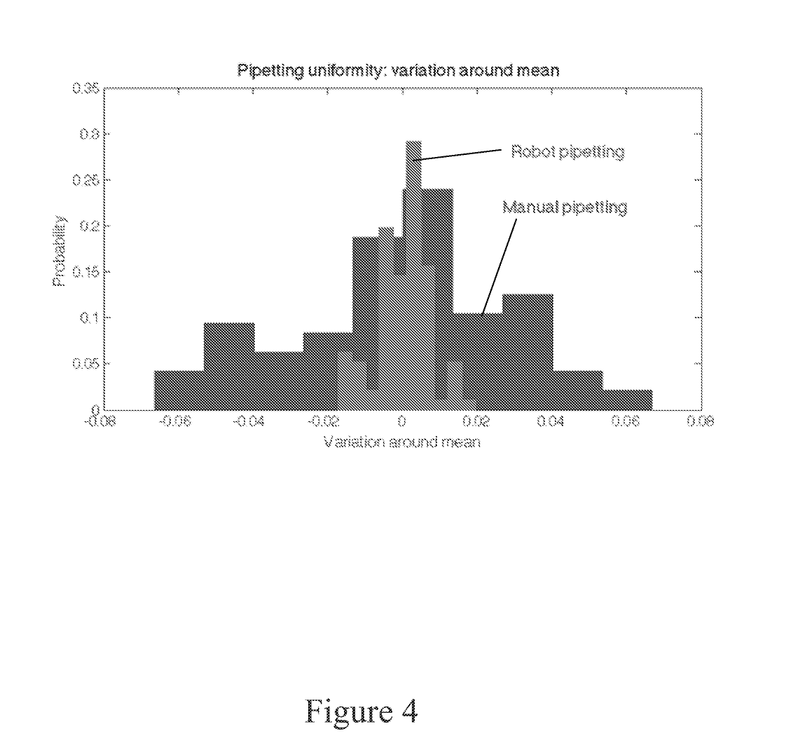

The fractional variations of manual vs. robotic pipetting were compared in a normalized histogram in FIG. 4. By eye, robotic pipetting is more uniform than manual pipetting. Quantitatively, we expressed this uniformity by normalizing the standard deviation of each plate's 96 optical density values by the average value of each plate. The normalized standard deviation of values using manual pipetting is 0.0278, whereas the normalized standard deviation of values using robotic pipetting is 0.0072.

### Test 2: Comparing the Precision of BCA and Bradford Protein Concentration Assay Kits.

BCA and Bradford assays are two common tools for calculating the amount of free protein in a given solution. To determine the variability of these assays, we created protein stocks with known volumes of bovine serum albumin (BSA), in a two-fold dilution series of seven steps down from 100 micrograms per ml, with phosphate buffered saline (PBS) at pH 7.4 as the diluent. All samples were generated in triplicate, and assessed via both BCA and Bradford assays, to determine the natural variability of these assays on identical samples.

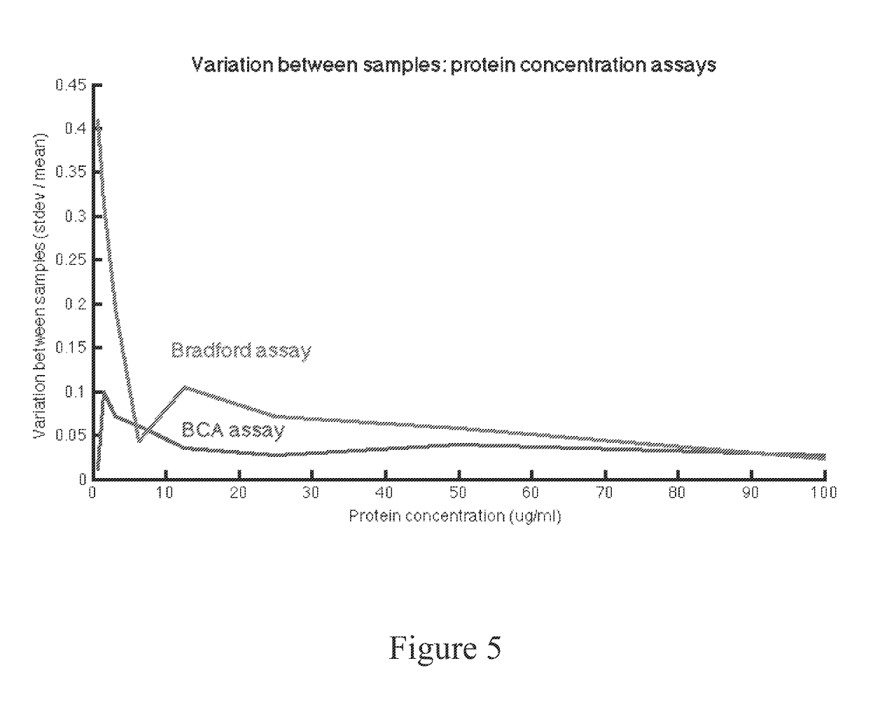

FIG. 5 shows the normalized variation between samples (standard deviation between each three identical samples, divided by the mean signal strength of the three samples), vs. the known initial concentration of each set of samples. From these data, we can determine that the Bradford and BCA assays are most accurate at protein concentrations above 5 micrograms per ml.

Test 3: Variation in Fluorescent Protein Expression Between Adjacent Wells with Identical Initial Cell Stocks.

Our initial two tests quantified the variability in optical readouts introduced by robotic vs. manual pipetting, and the precision of BCA and Bradford protein concentration assays. As protein constructs fused with fluorescent or luminescent protein domains provide a high-precision tool for estimating the amount of protein secreted by a given cell strain, we wished to explore the natural variability in fluorescent protein secretion by a 96-well plate cultured with identical amounts of cell stocks.

A 96-well plate was divided into 24-well quadrants, each of which was seeded with 200 microliters of dilute cell culture suspended in BMGY growth buffer; after 24 hours of cell growth, to an optical density of ˜2.0, protein expression and secretion was initiated by switching to a buffer containing the induction agent (in this case 0.5% methanol). The four quadrants were seeded with serial 4× dilutions of cell stock, starting with OD600 of ˜0.001.

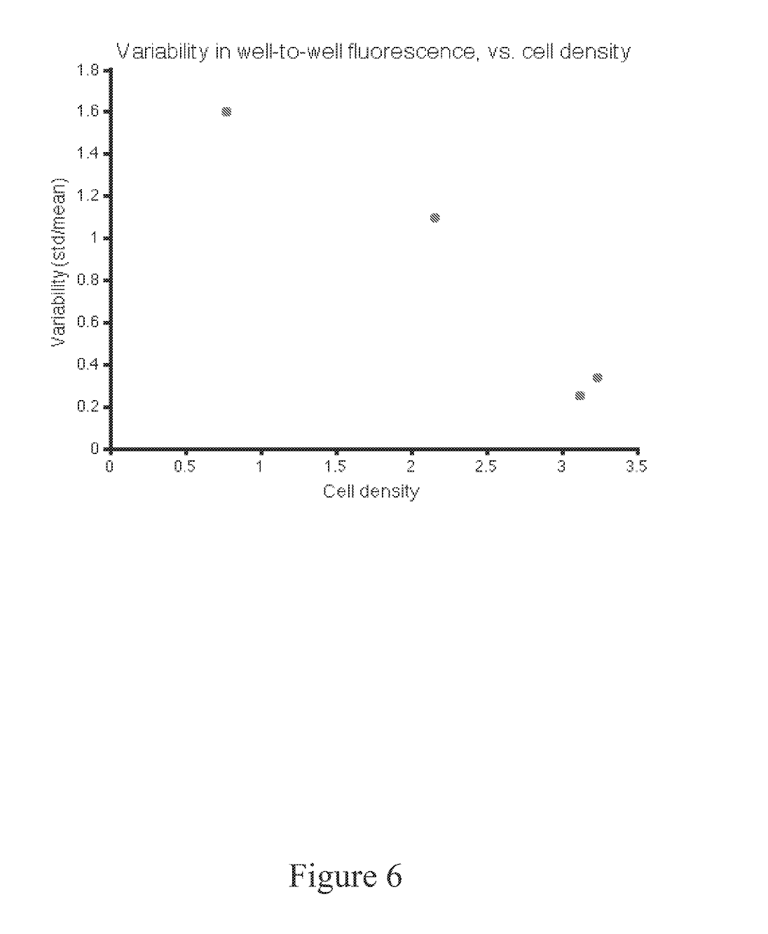

FIG. 6 shows the normalized variability of fluorescence signal vs. the average optical density (i.e., OD) of each quadrant. The clustering of the two highest ODs indicates that the two highest-density quadrants were equally saturated in terms of cell growth; it is also clear that to get a high signal-to-noise ratio (i.e. normalized variability below 0.5), cell densities should be above the OD600 range of ˜3.0.

### Test 4: Normalized Fluorescence Per Cell Density

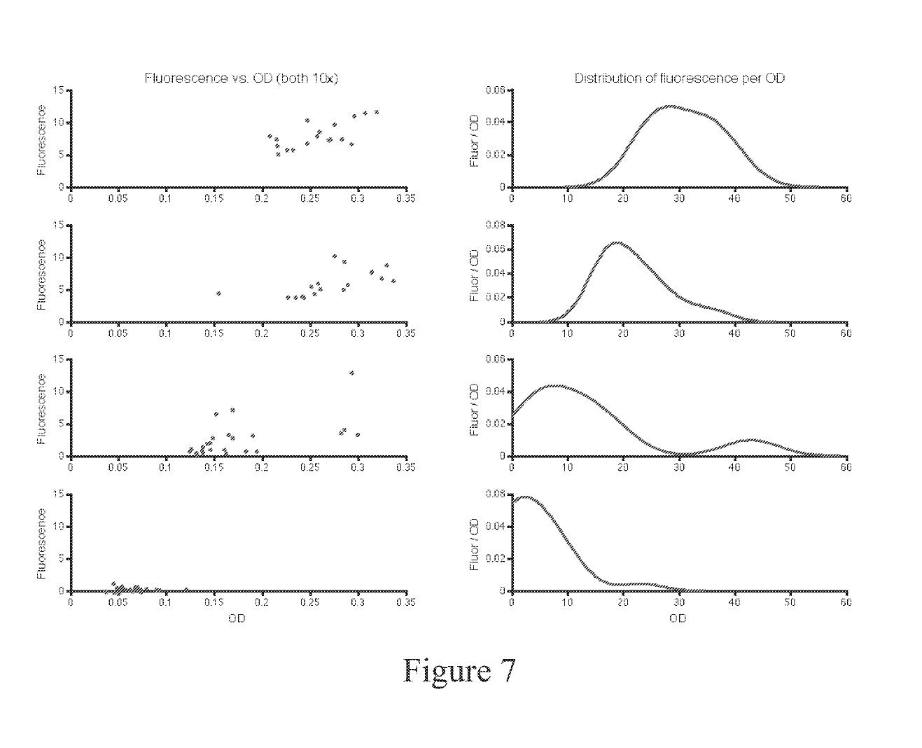

FIG. 7 shows scatterplots of fluorescence vs. raw optical density measurements for each quadrant's wells from Test 3, and the normalized fluorescence signal per optical density for each well. Quadrants 1-4 are in order of decreasing initial cell density (i.e., Quadrant 1 has the highest initial cell density, and Quadrant 4 has the least initial cell density). The spread in normalized fluorescence is consistent across three of the four quadrants (Table 2). The deviation in Quadrant 3 is due to a few significant outliers, as seen in FIG. 7.

TABLE 2
Mean and standard deviations of fluorescence,
normalized by cell density.

| | Quadrant 1 | Quadrant 2 | Quadrant 3 | Quadrant 4 |
|---|---|---|---|---|
| Mean | 31 | 22 | 14 | 3.2 |
| fluor./OD | | | | |
| St. dev. | 5.8 | 6.1 | 13 | 6.7 |
| fluor./OD | | | | |

### Test 5: Plate-to-Plate Uniformity Testing

Cell growth and protein production are sensitive to many factors, especially ambient levels of oxygen and humidity. When culturing many plates in an incubator, it therefore becomes crucial to ensure that plates are stacked with sufficient space between one another to allow for sufficient oxygenation of all plates, and to ensure that any unavoidable variation across different plate locations in a stack within an incubator is well understood. One primary comparison to make is between plates on the top of stacks, and plates below them, which will likely have different amounts of dissolved oxygen in their culture volumes—and potentially even within adjacent wells of plates that are in the middle or on the bottom of a stack.

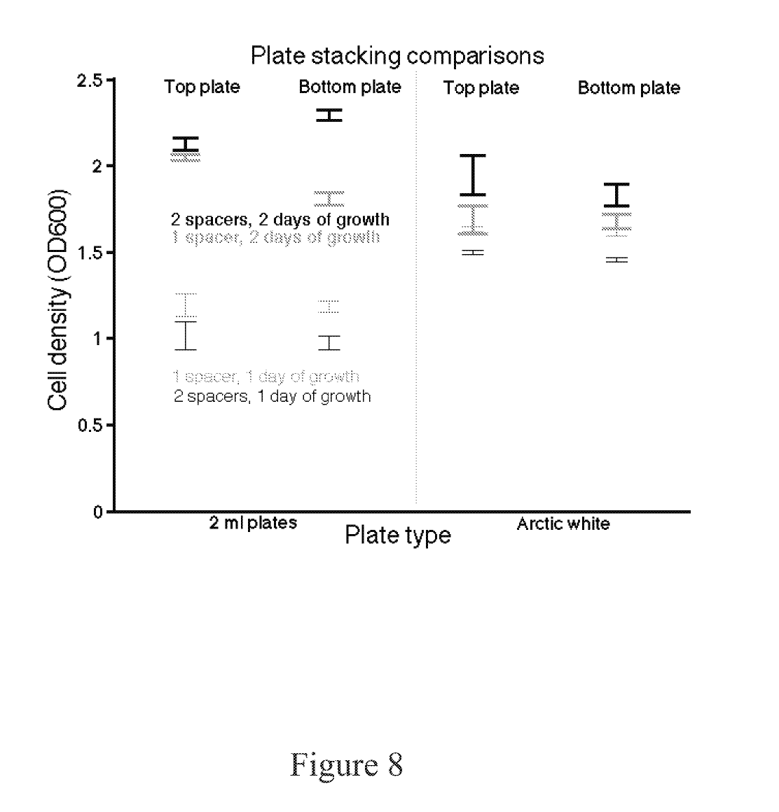

FIG. 8 shows the cell densities achieved after one and two days of growth in several different pairs of conditions: using two different plate types (1 ml and 2 ml plate volumes); with two plates of each type stacked on top of one another, to assess whether a plate on top of a stack grows faster than one on the bottom of a stack; and with one or two plastic spacers creating a gap between two plates, to determine if an increase in the gap between two stacked plates causes a clear change in the cells' growth rate. Error bars indicate the standard deviation of values measured across eight wells with identical culture volumes and initial cell densities in each plate.

Comparing the two plate types after one day of growth (thin lines), the 1 ml plates appear to reach saturation faster than 2 ml plates; however, once they reach saturation (after two days of growth), both plate volumes reach similar cell densities. Comparing growth across spacer numbers, the data do not indicate a significant difference in trend between top and bottom plates in each stack (if the spacers have a significant effect, they should only do so for the bottom plates, as the top plates have nothing covering them). Top and bottom plates also appear to have similar growth characteristics, so it appears that oxygenation is not a significant issue when at least one spacer is present to permit air flow to plates on the bottom of a stack.

### Example 5

### Improvement of Lycopene Production in Pichia pastoris

Generation of a Pichia pastoris Strain that Produces Lycopene

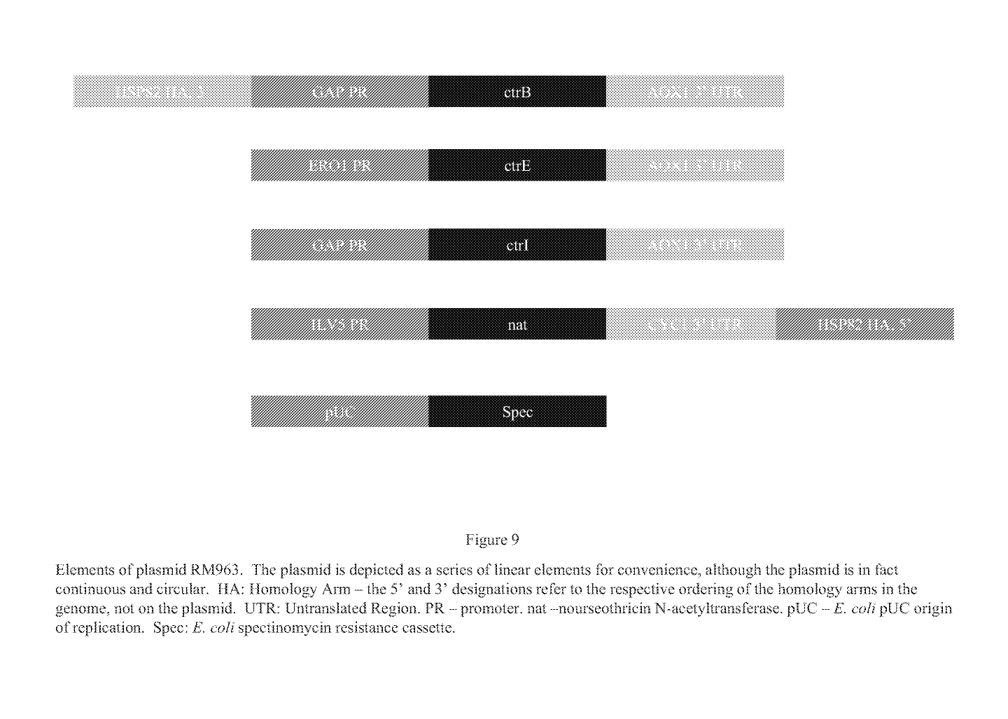

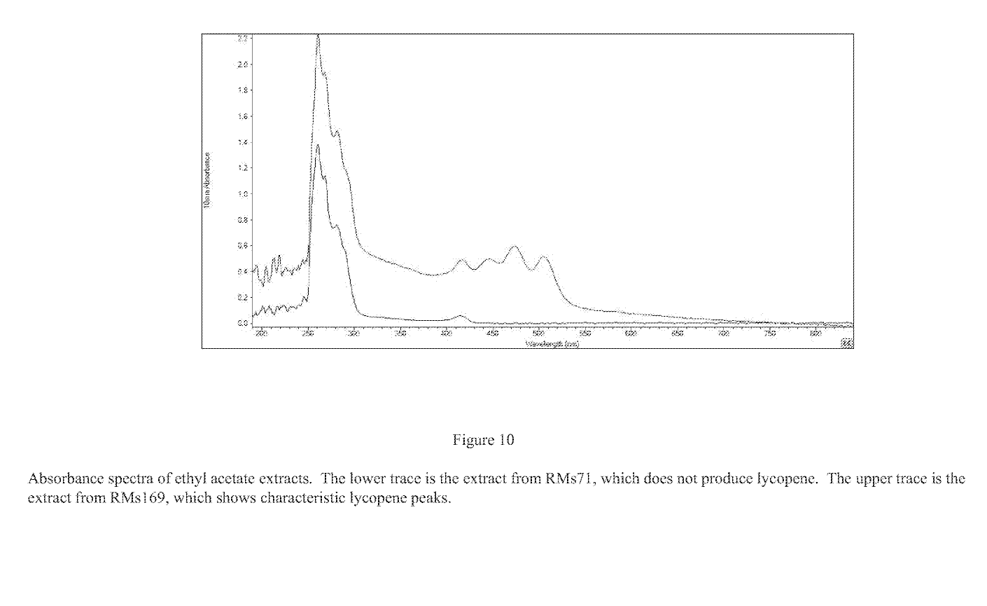

Biosynthesis of the carotenoid lycopene in Pichia pastoris requires introduction of three enzymes, geranylgeranyl diphosphate synthase (CrtE), phytoene synthase (CrtB), and phytoene desaturase (CrtI), as suggested by Ausich et al. (Ausich et al., 1996) and demonstrated by Bhataya et al. (Bhataya et al., 2009). Accordingly, plasmid RM963 (SEQ ID NO: 1, diagrammed in FIG. 9) was synthesized to include all of the elements necessary for expression of CrtB, CrtE, and CrtI in Pichia pastoris. Digestion of RM963 with BsaI followed by transformation into strain RMs71 (Strain GS115—NRRL Y15851—with the mutation in the HIS4 locus restored to the wild type sequence of NRRLY 11430 by transformation with linear double-stranded DNA having the sequence of SEQ ID NO: 2 followed by growth on media lacking histidine) according to the method of Wu and Letchworth (Wu and Letchworth, 2004) and selection on nourseothricin containing agar plates results in integration of the expression cassettes into the HSP82 locus. Colonies resulting from this transformation (strain RMs169) show a distinct reddish color, indicating the biosynthesis of lycopene. The presence of lycopene was confirmed by ethyl acetate extraction: a colony of RMs169 and a colony of RMs71 (non lycopene producing strain) were each used to inoculate 50 ml of YPD. After growth for 48 hours, each culture was pelleted by centrifugation, the supe discarded, and the cells resuspended in 15 ml of water containing 20 units of lyticase. After incubation for 1 hour at 37° C., the cultures were sonicated, mixed with 7 ml of ethyl acetate, vortexed, then centrifuged. The organic layer was extracted and the absorbance spectrum collected (FIG. 10). The extract of RMs169 shows characteristic lycopene peaks at 443, 471, and 502 nm, while the extract of RMs71 shows no peaks at the corresponding wavelengths.

TABLE 3
Vector and Linear Sequences

| Name | SEQ ID NO: |
|---|---|
| RM963 | 1 |
| HIS4 restoration | 2 |
| RM919 | 3 |
| RM921 | 4 |
| RM991 | 5 |
| RM922 | 6 |

### Construction of a Reprogramming Library

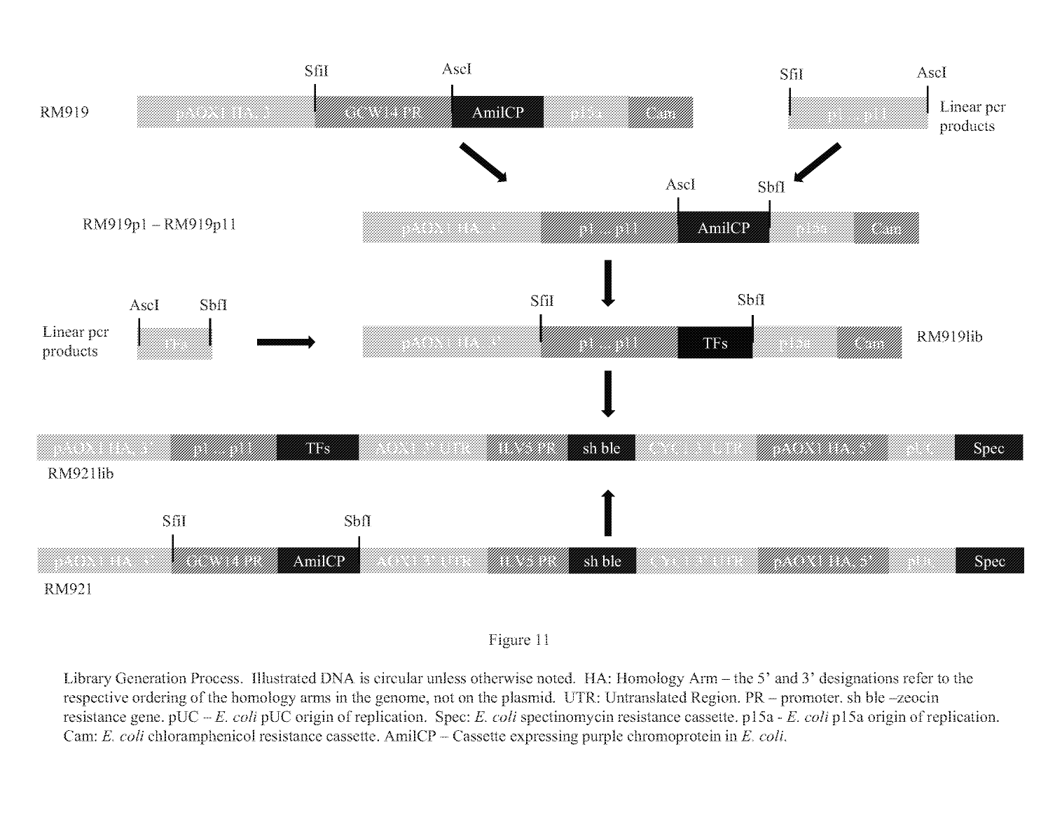

A library consisting of 11 promoters operably linked to each of 96 putative regulatory elements (total theoretical diversity of 1056 combinations) was generated to validate the ability of a reprogramming library to improve desired cellular phenotypes. The library synthesis process is diagrammed in FIG. 11. The 11 promoters listed in Table 4 were first amplified from the genome of Pichia pastoris strain GS115 (NRRL Y15851). Each reaction consisted of 5 μL 5×HF Phusion Buffer, 0.25 μl Phusion Polymerase, 0.5 μM 10 μM forward oligo, 0.5 μl 10 μM reverse oligo, 5 ng template DNA (GS115 genomic DNA), 0.5 μl of 10 mM dNTPs, and ddH2O added to final volume of 25 μl. The reaction was then thermocycled according to the program:

1. Denature at 94° C. for 5 minutes
2. Denature at 94° C. for 30 seconds
3. Anneal at 55° C. for 30 seconds
4. Extend at 72° C. for 60 seconds
5. Repeat steps 2-4 for 29 additional cycles
6. Final extension at 72° C. for 5 minutes

TABLE 4
Oligonucleotide sequences for amplifying promoters
p1-p11, and resulting promoter sequences

| | | | | Sequence |
|---|---|---|---|---|
| | | | | (5′ → 3′) |
| | | | | including intro- |
| | | F Oligo | R Oligo | duced flanking |
| | | (5′ → 3′) | (5′ → 3′) | restriction sites, |
| Name | ORF 3′ of Promoter | SEQ ID NO: | SEQ ID NO: | SEQ ID NO: |
| p1 | PAS_chr1-1_0107 | 7 | 18 | 29 |
| p2 | PAS_chr1-4_0299 | 8 | 19 | 30 |
| p3 | PAS_chr3_0647 | 9 | 20 | 31 |
| p4 | PAS_chr4_0112 | 10 | 21 | 32 |
| p5 | PAS_chr4_0785 | 11 | 22 | 33 |
| p6 | PAS_chr3_1011 | 12 | 23 | 34 |
| p7 | PAS_chr2-1_0428 | 13 | 24 | 35 |
| p8 | PAS_chr1-4_0426 | 14 | 25 | 36 |
| p9 | PAS_chr4_0720 | 15 | 26 | 37 |
| p10 | PAS_chr2-2_0067 | 16 | 27 | 38 |
| p11 | PAS_chr2-1_0437 | 17 | 28 | 39 |

For p6 (SEQ ID NO: 12), DMSO (final concentration 4% v/v) was added to the reaction. After amplification, the DNA was separated on an agarose gel and the ˜1000 bp band extracted, then cloned into plasmid RM919 (SEQ ID NO: 3) via digestion with SfiI and AscI, resulting in 11 distinct plasmids (RM919p1-RM919p11). 500 ng of each of the 11 plasmids was digested with AscI and SbfI and then gel purified to extract the ˜3500 bp fragment. The digested vectors were then pooled (RM919pool).

A set of 96 elements was randomly selected from the list of putative regulatory elements listed in Table 1 and other predicted regulators. The putative regulatory elements were PCR amplified from the GS115 (NRRL Y15851) genome using the primers listed in Table 5. The polymerase reaction was identical to the one described above for amplification of the promoters, with the exception of regulatory element numbers 11, 20, 22, 26, 32, 35, 39, 45, 51, 65, 81, 83, and 92 of Table 5, which were amplified using the following program:

1. Denature at 94° C. for 5 minutes
2. Denature at 94° C. for 30 seconds
3. Anneal at 55° C. for 30 seconds
4. Extend at 72° C. for 240 seconds
5. Repeat steps 2-4 for 29 additional cycles
6. Final extension at 72° C. for 5 minutes

TABLE 5
Oligonucleotide sequences for amplifying
putatitive regulatory elements

| | | F Oligo | R Oligo |
|---|---|---|---|
| | | (5′ → 3′) | (5′ → 3′) |
| Number | Sequence Identifier | SEQ ID NO: | SEQ ID NO: |
| 1 | XM_002494290.1 | 40 | 136 |
| 2 | XM_002493563.1 | 41 | 137 |
| 3 | XM_002493526.1 | 42 | 138 |
| 4 | XM_002490282.1 | 43 | 139 |
| 5 | XM_002491699.1 | 44 | 140 |
| 6 | XM_002493323.1 | 45 | 141 |
| 7 | XM_002490851.1 | 46 | 142 |
| 8 | XM_002490293.1 | 47 | 143 |
| 9 | XM_002490399.1 | 48 | 144 |
| 10 | XM_002493170.1 | 49 | 145 |
| 11 | XM_002491183.1 | 50 | 146 |
| 12 | CAY67026.1 | 51 | 147 |
| 13 | XM_002492126.1 | 52 | 148 |
| 14 | XM_002491802.1 | 53 | 149 |
| 15 | XM_002492077.1 | 54 | 150 |
| 16 | XM_002493528.1 | 55 | 151 |
| 17 | XM_002491607.1 | 56 | 152 |
| 18 | XM_002489552.1 | 57 | 153 |
| 19 | XM_002494115.1 | 58 | 154 |
| 20 | XM_002492101.1 | 59 | 155 |
| 21 | XM_002491374.1 | 60 | 156 |
| 22 | XM_002490926.1 | 61 | 157 |
| 23 | XM_002489994.1 | 62 | 158 |
| 24 | XM_002492744.1 | 63 | 159 |
| 25 | XM_002494212.1 | 64 | 160 |
| 26 | XM_002490355.1 | 65 | 161 |
| 27 | XM_002490819.1 | 66 | 162 |
| 28 | XM_002490965.1 | 67 | 163 |
| 29 | XM_002493832.1 | 68 | 164 |
| 30 | XM_002489855.1 | 69 | 165 |
| 31 | XM_002492110.1 | 70 | 166 |
| 32 | XM_002491173.1 | 71 | 167 |
| 33 | XM_002491672.1 | 72 | 168 |
| 34 | XM_002489306.1 | 73 | 169 |
| 35 | XM_002489678.1 | 74 | 170 |
| 36 | XM_002493699.1 | 75 | 171 |
| 37 | XM_002491226.1 | 76 | 172 |
| 38 | XM_002492738.1 | 77 | 173 |
| 39 | XM_002489653.1 | 78 | 174 |
| 40 | XM_002491017.1 | 79 | 175 |
| 41 | XM_002493553.1 | 80 | 176 |
| 42 | XM_002491909.1 | 81 | 177 |
| 43 | XM_002490682.1 | 82 | 178 |
| 44 | XM_002493851.1 | 83 | 179 |
| 45 | XM_002491711.1 | 84 | 180 |
| 46 | XM_002489841.1 | 85 | 181 |
| 47 | XM_002490432.1 | 86 | 182 |
| 48 | XM_002490417.1 | 87 | 183 |
| 49 | XM_002493834.1 | 88 | 184 |
| 50 | XM_002491260.1 | 89 | 185 |
| 51 | XM_002490735.1 | 90 | 186 |
| 52 | XM_002490613.1 | 91 | 187 |
| 53 | XM_002491761.1 | 92 | 188 |
| 54 | XM_002491220.1 | 93 | 189 |
| 55 | XM_002492657.1 | 94 | 190 |
| 56 | XM_002489422.1 | 95 | 191 |
| 57 | XM_002489917.1 | 96 | 192 |
| 58 | XM_002491250.1 | 97 | 193 |
| 59 | XM_002493392.1 | 98 | 194 |
| 60 | XM_002493377.1 | 99 | 195 |
| 61 | XM_002489633.1 | 100 | 196 |
| 62 | XM_002493454.1 | 101 | 197 |
| 63 | XM_002490476.1 | 102 | 198 |
| 64 | XM_002492717.1 | 103 | 199 |
| 65 | XM_002493710.1 | 104 | 200 |
| 66 | XM_002490833.1 | 105 | 201 |
| 67 | XM_002491859.1 | 106 | 202 |
| 68 | XM_002493398.1 | 107 | 203 |
| 69 | XM_002491123.1 | 108 | 204 |
| 70 | XM_002491626.1 | 109 | 205 |
| 71 | XM_002491403.1 | 110 | 206 |
| 72 | XM_002489650.1 | 111 | 207 |
| 73 | XM_002491952.1 | 112 | 208 |
| 74 | XM_002490082.1 | 113 | 209 |
| 75 | XM_002490629.1 | 114 | 210 |
| 76 | XM_002491645.1 | 115 | 211 |
| 77 | XM_002490198.1 | 116 | 212 |
| 78 | XM_002490795.1 | 117 | 213 |
| 79 | XM_002490105.1 | 118 | 214 |
| 80 | XM_002493281.1 | 119 | 215 |
| 81 | XM_002489525.1 | 120 | 216 |
| 82 | XM_002493237.1 | 121 | 217 |
| 83 | XM_002489482.1 | 122 | 218 |
| 84 | XM_002492403.1 | 123 | 219 |
| 85 | XM_002490606.1 | 124 | 220 |
| 86 | XM_002491910.1 | 125 | 221 |
| 87 | XM_002490170.1 | 126 | 222 |
| 88 | XM_002490608.1 | 127 | 223 |
| 89 | XM_002494020.1 | 128 | 224 |
| 90 | XM_002492342.1 | 129 | 225 |
| 91 | XM_002490329.1 | 130 | 226 |
| 92 | XM_002492458.1 | 131 | 227 |
| 93 | XM_002490253.1 | 132 | 228 |
| 94 | XM_002492996.1 | 133 | 229 |
| 95 | XM_002490065.1 | 134 | 230 |
| 96 | XM_002493268.1 | 135 | 231 |

The resulting PCR products were separated by agarose gel electrophoresis, and the desired products extracted and pooled. After gel extraction, 6.4 μg of the pooled PCR products were digested with AscI and SbfI. After cleanup, the digested regulatory element DNA was ligated to the digested promoter vectors, RM919pool. The resulting ligation products were transformed into E. coli strain MC1061 according to the manufacturer's instructions (Lucigen Corp., catalog #60514-1) and plated on chloramphenicol containing agar plates. After incubation for 16 hours at 37° C., cells were pooled and DNA extracted, resulting in RM919lib.

Finally, the promoter-regulatory elements pairs of RM919lib were transferred to RM921 (SEQ ID NO: 4), which contains the elements necessary for replication in E. coli and integration into the genome of Pichia pastoris at the pAOX1 locus. 6.4 μg of RM919lib was digested with SbfI and SfiI before cleanup, and 6.2 μg of RM921 was digested with SbfI and SfiI before agarose gel separation and extraction of the ˜4700 bp fragment. The digested RM919lib and RM921 DNA was ligated and transform into E. coli strain MC1061 according to the manufacturer's instructions (Lucigen Corp., catalog #60514-1) and plated on spectinomycin containing agar plates. After incubation for 16 hours at 37° C., cells were pooled and DNA extracted, resulting in RM921lib.

Introduction of the Reprogramming Library into the Lycopene Producing Strain of Pichia pastoris and Identification of Improved Clones

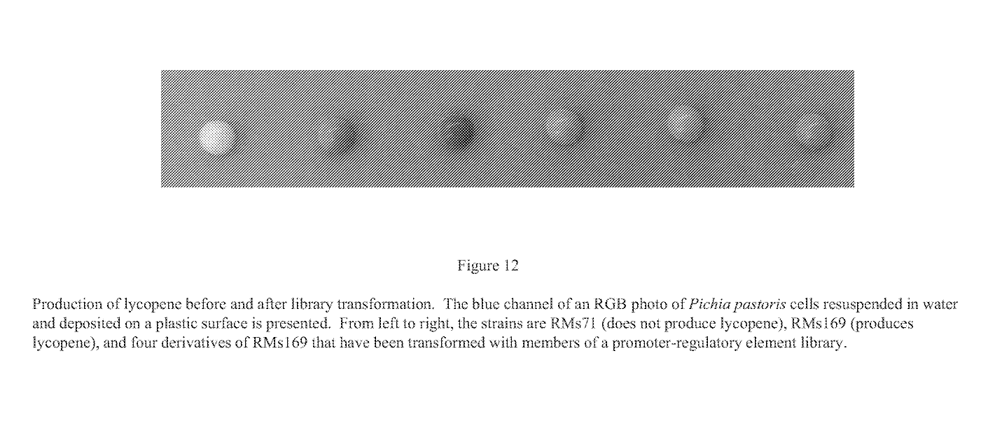

The RM921lib DNA was digested with PmeI before transformation into RMs169 according to the method of Wu and Letchworth (Wu and Letchworth, 2004). Transformants were plated on agar plates containing zeocin at 100 μg/ml and incubated for 48 hours at 30° C., followed by 48 hours of incubation at room temperature. Approximately 10,000 colonies were visually inspected, and 16 clones exhibiting apparently darker red coloration were selected for further analysis, streaked onto fresh agar plates, and incubated for 48 hours at 30° C. The four clones with the darkest red coloration (by visual inspection), a colony of RMs169 (lycopene producing strain without any transformed library member), and a colony of RMs71 (non lycopene producing strain) were each used to inoculate 50 ml of YPD. After growth for 48 hours, each culture was pelleted by centrifugation, the supe discarded, the cells resuspended in 20 ml of water, and 5 μl deposited on a plastic surface (FIG. 12). The first library member containing clone (3^{rd} spot from the left) appears much more visually red than the untransformed clone (2^{nd} spot from the left), indicating improved production of lycopene, and confirms that even a relatively small promoter-regulator library (˜1000 members) is capable of improving production of a small molecule in Pichia pastoris.

### Example 6

### Improvement of Secretion of Silk Polypeptide—Green Fluorescent Protein (GFP) Fusion in Pichia pastoris

Generation of a Pichia pastoris Strain that Secretes a Silk Polypeptide—GFP Fusion

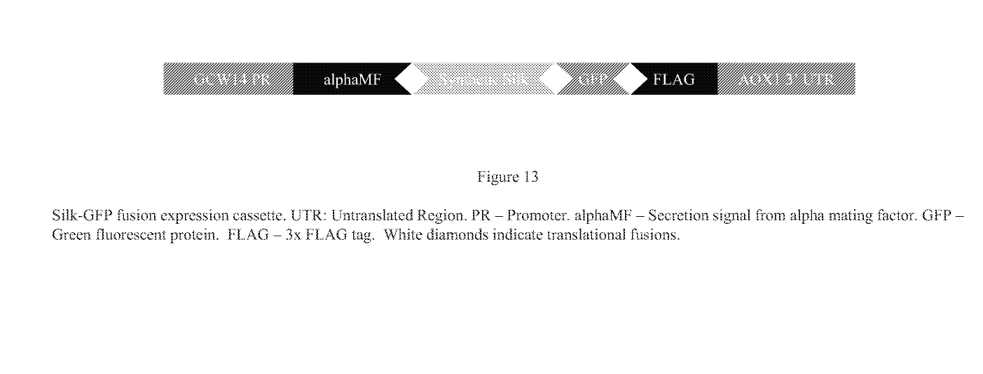

Major ampullate (dragline) spider silk exhibits excellent mechanical properties, and is therefore of interest to express recombinantly. The structural silk genes that form the dragline of Argiope bruennichi (AB MaSp1 and AB MaSp2) have recently been sequenced (Zhang et al., 2013). To circumvent the challenges of expressing the native MaSp polypeptides, a shorter synthetic sequence was designed that captures important features of the full-length AB MaSp2 sequence (Synthetic Silk). Further, to enable facile detection of the synthetic silk protein, a green fluorescent protein (GFP) bearing a C-terminal tag (3× FLAG) was translationally fused to the silk's C-terminus. A yeast secretion signal (from alpha mating factor—αMF) was then fused to the N-terminus of the silk-GFP fusion to cause secretion of the polypeptide. The αMF-silk-GFP construct was placed under the transcriptional control of a strong constitutive promoter, P_{GCW14} (Liang et al., 2013), with transcription terminated by a sequence from the 3′ UTR (untranslated region) of the AOX1 locus (FIG. 13). The expression cassette was then cloned into three different vectors, each of which integrates into a different locus and expresses a different dominant resistance marker or restores a different biosynthetic pathway (Table 6). The αMF-silk-GFP construct was integrated into three locations of the genome of Pichia pastoris strain GS115 (NRRL Y15851) by transforming in each of the three vectors (RM848, RM850, and RM851), following digestion with BsaI, using the method of Wu and Letchworth (Wu and Letchworth, 2004).

TABLE 6
Plasmids used for expression of silk-GFP fusion

| Plasmid | | | Sequence including silk-GFP |
|---|---|---|---|
| Name | Marker | Locus | cassette SEQ ID NO: |
| RM848 | Restores HIS4 | HIS4 | 232 |
| RM850 | Nourseothricin | HSP82 | 233 |
| RM851 | Hygromycin | TEF1 | 234 |

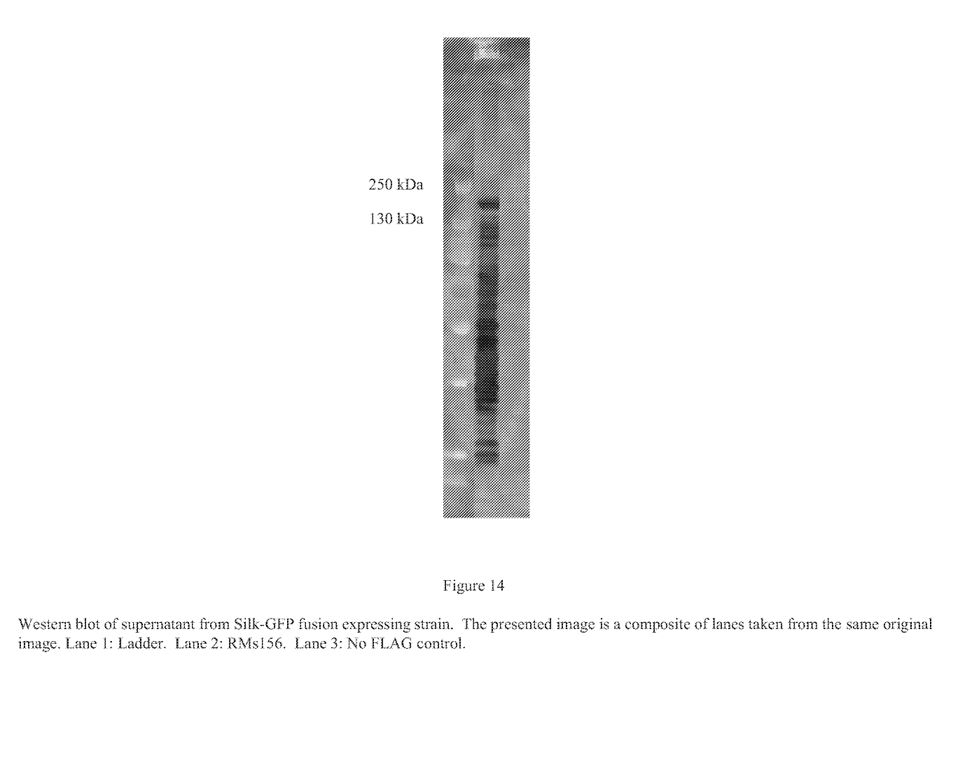

Secretion of silk-GFP from the resulting strain, RMs156, was confirmed by both western blot and fluorescence measurement of culture supernatant. A western blot (targeting the FLAG epitope) is shown in FIG. 14. While a strain transformed with an expression cassette lacking the 3×FLAG tag shows no significant signal (lane 3), strain RMs156 (lane 2) generated several detectable bands. The ladder of bands for RMs156 is presumed to be due to degradation products. The topmost band has an apparent molecular weight of ˜150 kDa, while the predicted molecular weight of the processed polypeptide is ˜110 kDa. Although the source of this discrepancy is unknown, other silk polypeptides have also been observed to appear at a higher than expected molecular weight. The fluorescence of the culture supernatant was also measured. First, isolated colonies (n=5) of both RMs71 (see Example 5) and RMs156 were used to inoculate 400 ul of BMGY in a 1 ml square-well, deep-well block. After incubation for 24 hours at 1000 rpm and 30° C., the OD600 was recorded, then the cells were pelleted by centrifugation and the supernatant collected. Subsequently, 50 μl of supernatant was mixed with 200 μl of 1M HEPES (pH 8.0), and the fluorescence (excitation: 490 nm, emission 519 nm) recorded. Strain RMs71 exhibited a mean OD-normalized fluorescence of 0.79, with a standard deviation of 0.07, while strain RMs156 exhibited a mean OD-normalized fluorescence of 21.56, with a standard deviation of 6.27. This confirms the secretion of a GFP containing polypeptide into the supernatant, consistent with the western blot data.

Introduction of Reprogramming Library into Silk-GFP Producing Strain and Identification of Improved Clones

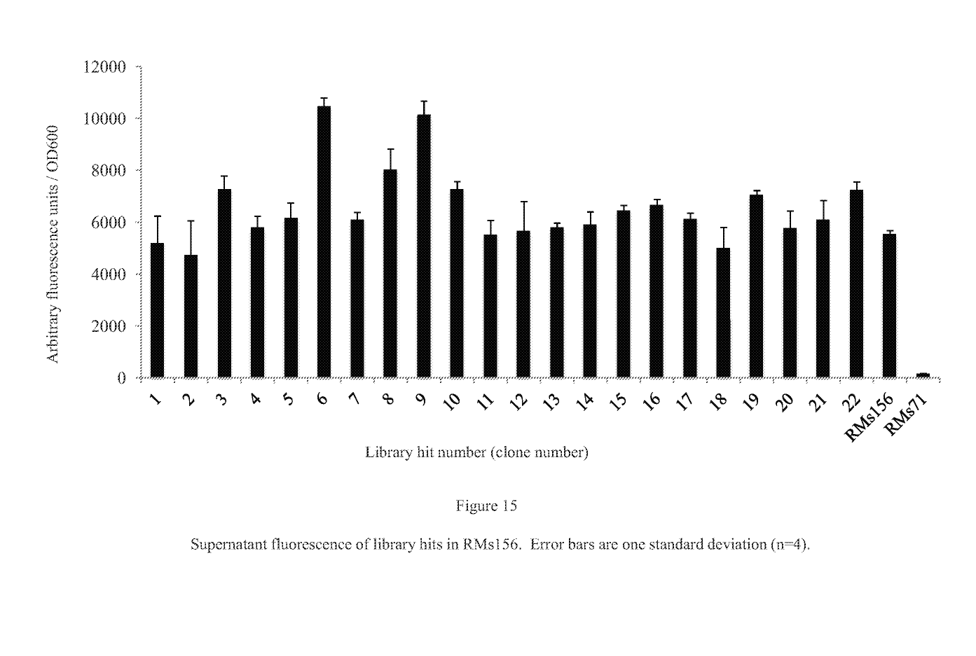

The RM921lib DNA (see Example 5) was digested with PmeI before transformation into RMs156 according to the method of Wu and Letchworth (Wu and Letchworth, 2004). Transformants were plated on agar plates containing zeocin at 100 μg/ml and incubated for 48 hours at 30° C. From the resulting colonies, 2000 were randomly selected to inoculate 400 μl of YPD media in a 1 ml square-well, deep-well block. After 48 hours of growth at 30° C. and 1000 rpm, the fluorescence of the cells in culture was measured. The 22 clones exhibiting the highest fluorescence signal were streaked out for further analysis. Isolated colonies (n=4) of each of the 22 clones, RMs71, and RMs156 were used to inoculate 400 μl of BMGY in a 1 ml square-well, deep-well block. After incubation for 48 hours at 1000 rpm and 30° C., the OD600 was recorded, then the cells were pelleted by centrifugation and the supernatant collected. Subsequently, 50 μl of supernatant was mixed with 200 μl of 1M HEPES (pH 8.0), and the fluorescence (excitation: 490 nm, emission 519 nm) recorded. FIG. 15 shows the resulting OD-normalized fluorescence values. Two clones, clone 6 and clone 9, show ˜1.8 fold increased fluorescence compared to RMs156. This confirms that a relatively small promoter-regulator library (˜1000 members) is capable of improving production of a silk-GFP fusion in Pichia pastoris.

### Example 7

### Improvement of Intracellular GFP Production in Saccharomyces cerevisiae

Generation of a Saccharomyces cerevisiae Strain that Produces Intracellular GFP

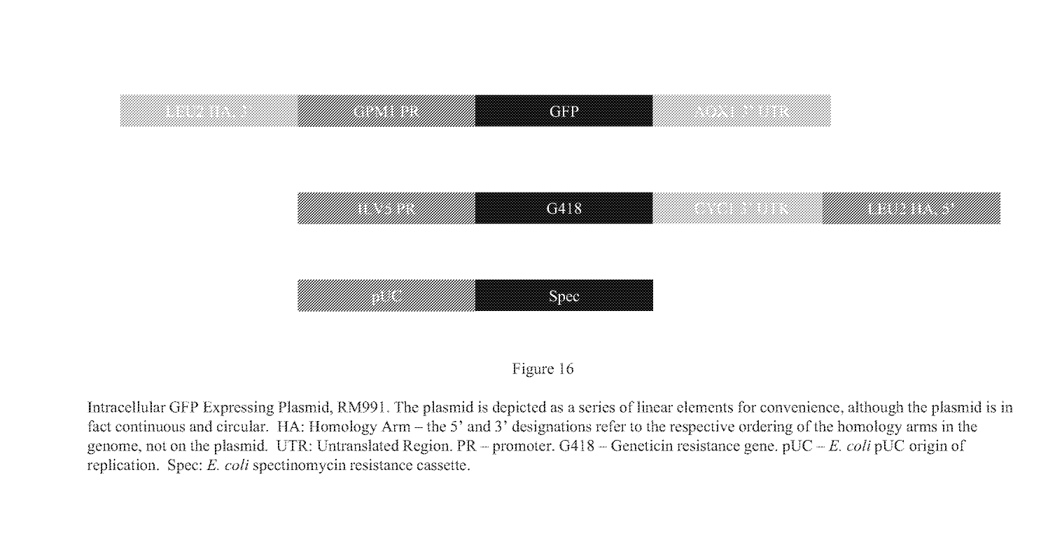

Saccharomyces cerevisiae strain s288c was transformed with plasmid RM991 (SEQ ID NO: 5) linearized with BsaI to produce a strain that expresses intracellular GFP. RM991 is diagrammed in FIG. 16, and contains promoter P_{GPM1} driving expression of GFP, as well as sequences targeting the LEU2 locus and a cassette that expresses resistance to G418 (Geneticin). Resulting colonies, strain RMs176, and colonies of s288c, were used to inoculate 5 ml of YPD in 12 ml culture tubes and incubated at 30° C. for 24 hours with agitation at 300 rpm. The OD600 was measured, and the fluorescence (excitation 470 nm, emission 512 nm) recorded. Strain RMs176 exhibited an OD-normalized fluorescence of 3.0, while strain s288c exhibited an OD-normalized fluorescence of 10.5. This confirms production of green fluorescent protein by strain RMs176.

### Construction of a Reprogramming Library

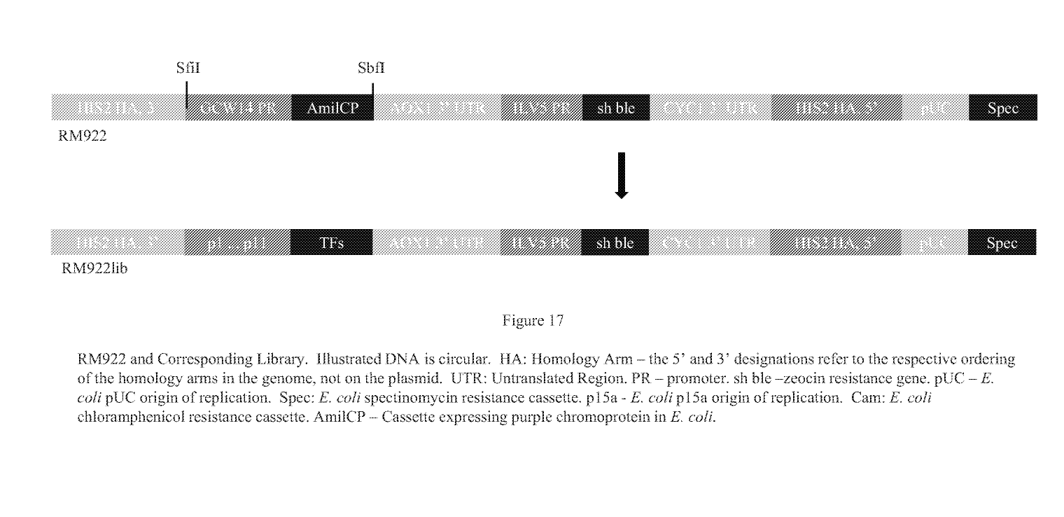

The promoter-regulatory elements pairs of RM919lib (see Example 5) were transferred to RM922 (SEQ ID NO: 6), which contains the elements necessary for replication in E. coli and integration into the genome of Saccharomyces cerevisiae at the HIS2 locus (FIG. 17). 6.4 μg of RM919lib was digested with SbfI and SfiI before cleanup, and 6.2 μg of RM922 was digested with SbfI and SfiI before gel purification and extraction of the ˜5400 bp fragment. The digested RM919lib and RM922 DNA was ligated and transform into E. coli strain MC1061 according to the manufacturer's instructions (Lucigen Corp., catalog #60514-1) and plated on spectinomycin containing agar plates. After incubation for 16 hours at 37° C., cells were pooled and DNA extracted, resulting in RM922lib.

Introduction of the Reprogramming Library into the GFP Producing Strain and Identification of Improved Clones

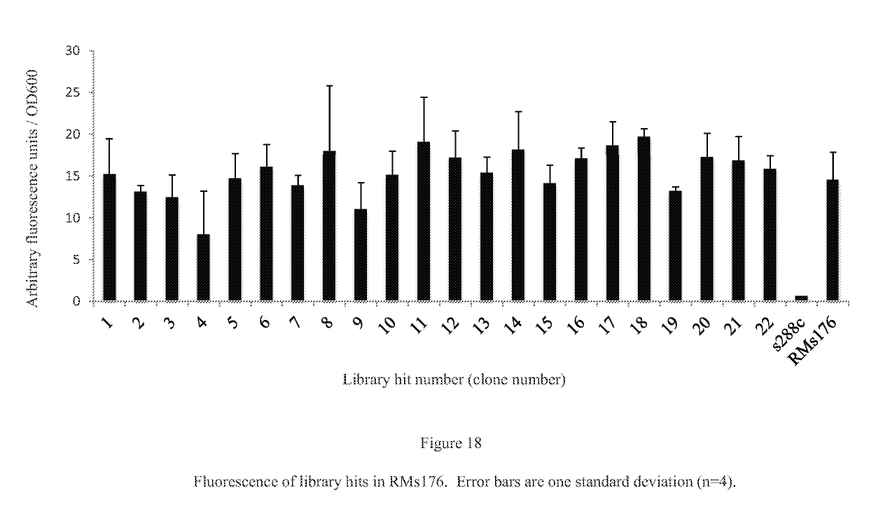

The RM922lib DNA was digested with SwaI before transformation into RMs176. Transformants were plated on agar plates containing zeocin at 100 μg/ml and incubated for 48 hours at 30° C. From the resulting colonies, 2000 were randomly selected to inoculate 400 μl of YPD media in a 1 ml square-well, deep-well block. After 48 hours of growth at 30° C. and 1000 rpm, the fluorescence of the cells in culture was measured. The 22 clones exhibiting the highest fluorescence signal were streaked out for further analysis. Isolated colonies (n=4) of each of the 22 clones, s288c, and RMs176 were used to inoculate 400 μl of YPD in a 1 ml square-well, deep-well block. After incubation for 42 hours at 1000 rpm and 30° C., the cells were pelleted by centrifugation and the supernatant discard. The cells were resuspended in 500 μl PBS, pelleted by centrifugation, and the supernatant again discarded. After resuspension in 400 μl PBS, the OD600 was recorded, and the fluorescence measured (excitation 470 nm, emission 512 nm). FIG. 18 shows the resulting fluorescence measurements. Library clone 18 shows ˜1.4 fold increased OD normalized fluorescence compared to RMs176, with the difference being statistically significant by one tailed t-test (p<0.05). This demonstrates that a relatively small promoter-regulator library (˜1000 members) is capable of improving production of an intracellular protein in Saccharomyces cerevisiae.

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

The language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

All references, issued patents and patent applications cited within the body of the instant specification are hereby incorporated by reference in their entirety, for all purposes.

### REFERENCES

Aper, Hal, et al., (2006) Engineering Yeast Transcription Machinery for Improved Ethanol Tolerance and Production Science 314, 1565.
Ausich, R. L., Brinkhaus, F. L., Mukharji, I., Proffitt, J., Yarger, J., Yen, H.-C. B., 1996. Lycopene biosynthesis in genetically engineered hosts. U.S. Pat. No. 5,530,189 A.
Bhataya, A., Schmidt-Dannert, C., Lee, P. C., 2009. Metabolic engineering of Pichia pastoris X-33 for lycopene production. Process Biochemistry 44, 1095-1102.
Cho, H. and Cronan, J. E. (1993) The Journal of Biological Chemistry 268: 9238-9245.
Chollet, R et al. (2004) Antimicrobial Agents and Chemotherapy 48: 3621-3624.
Gibson, D. G., et al., (2009) Enzymatic assembly of DNA molecules up to several hundred kilobases. Nature methods, 6(5).
Kalscheuer, R., et al. (2006a) Microbiology 152: 2529-2536.
Kalscheuer, R. et al. (2006b) Applied and Environmental Microbiology 72: 1373-1379.
Kameda, K. and Nunn, W. D. (1981) The Journal of Biological Chemistry 256: 5702-5707.
Liang, S., Zou, C., Lin, Y., Zhang, X., Ye, Y., 2013. Identification and characterization of P GCW14: a novel, strong constitutive promoter of Pichia pastoris. Biotechnol. Lett.
Lopez-Mauy et al., Cell (2002) v. 43:247-256
Nielsen, D. R et al. (2009) Metabolic Engineering 11: 262-273.
Qi et al., Applied and Environmental Microbiology (2005) v. 71: 5678-5684
Stöveken, T. et al. (2005) Journal of Bacteriology 187:1369-1376
Tsukagoshi, N. and Aono, R. (2000) Journal of Bacteriology 182: 4803-4810
Wu, S., Letchworth, G. J., 2004. High efficiency transformation by electroporation of Pichia pastoris pretreated with lithium acetate and dithiothreitol. BioTechniques 36, 152-154.
Zhang, W., et al. (2006) Enhanced Secretion of Heterologous Proteins in Pichia pastoris
Zhang, Y., Zhao, A.-C., Sima, Y.-H., Lu, C., Xiang, Z.-H., Nakagaki, M., 2013. The molecular structures of major ampullate silk proteins of the wasp spider, Argiope bruennichi: a second blueprint for synthesizing de novo silk. Comp. Biochem. Physiol. B, Biochem. Mol. Biol. 164, 151-158.

Following Overexpression of Saccharomyces cerevisiae Chaperone Proteins. Biotechnology progress, 22(4), 1090-1095.

1. A method of identifying a cell comprising an optimized functionality, comprising:
i. obtaining a population of cells, wherein said population comprises cells engineered to include a member of an expression cassette library, wherein said expression cassette library comprises N distinct promoter elements, and M distinct regulatory elements, and wherein the library comprises up to (N×M) distinct combinations of said promoter elements operably linked to said regulatory elements, wherein each member of said expression cassette library comprises at least one of said N promoter elements operably linked to at least one of said M regulatory elements; and
ii. screening the population of cells to identify said cell comprising said optimized functionality.

2. (canceled)

3. The method of claim 1, wherein said identified cell further comprises a recombinant gene operably linked to a promoter.

4.-9. (canceled)

10. The method of claim 3, wherein said recombinant gene encodes a silk protein.

11. The method of claim 3, wherein said recombinant gene encodes a protein fused to a detectable marker.

12. The method of claim 11, wherein said detectable marker is selected from the group consisting of: an epitope tag, a fluorescent protein, a firefly luciferase, and a beta galactosidase.

13. The method of claim 1, wherein said cell comprising said optimized functionality comprises a silk protein expressing gene operably linked to a recombinant AOX1 promoter.

14. The method of claim 1, wherein said cell comprising said optimized functionality further comprises a heterologous gene operably linked to a promoter.

15. The method of claim 14, wherein the heterologous gene comprises a secretion signal.

16. (canceled)

17. The method of claim 1, wherein said cell comprising said optimized functionality comprises a silk protein expressing gene operably linked to a constitutive promoter.

18. The method of claim 1, wherein said optimized functionality comprises an altered metabolic, regulatory, or signaling process in said cell comprising said optimized functionality as compared to an initial population of cells lacking said expression cassette.

19. The method of claim 1, wherein said optimized functionality comprises an increase in an expression level of a protein in said cell comprising said optimized functionality as compared to an expression level of said protein in an otherwise identical cell lacking said expression cassette.

20. The method of claim 1, wherein said optimized functionality comprises an increase in a secretion level of a protein from said cell as compared to a secretion level of said protein from an otherwise identical cell lacking said expression cassette.

21.-68. (canceled)

69. A library of expression cassettes, wherein said expression cassette library comprises N distinct promoter elements, and M distinct regulatory elements, and wherein the library comprises up to (N×M) distinct combinations of said promoter elements operably linked to said regulatory elements, wherein each member of said expression cassette library comprises at least one of said N distinct promoter elements operably linked to at least one of said M distinct regulatory elements.

70.-71. (canceled)

72. The library of expression cassettes of claim 69, wherein said N distinct promoter elements comprise a subset of all known promoter elements endogenous to the cell.

73. The library of expression cassettes of claim 69, wherein said N distinct promoter elements comprise promoter elements exogenous to said cell.

74. The library of expression cassettes of claim 69, wherein said N distinct promoter elements comprise synthetic promoter elements.

75.-76. (canceled)

77. The library of expression cassettes of claim 69, wherein said M distinct regulatory elements comprise a subset of all known regulatory elements endogenous to the cell.

78. The library of expression cassettes of claim 69, wherein said M distinct regulatory elements comprise regulatory elements exogenous to the cell.

79. The library of expression cassettes of claim 69, wherein said M distinct regulatory elements comprise synthetic regulatory elements.

80. The library of expression cassettes of claim 69, wherein said promoter element is a chimeric promoter.

81.-139. (canceled)


SEQUENCE LISTING (242 sequences)

SEQ ID NO: 1 - DNA, 11260 bp, Artificial Sequence
  Description of Artificial Sequence Synthetic polynucleotide
gcgatcgcggtctcacagatgacagagttgtcaagaacttgaccactttgttgttcgacacagctttgttgacttccggtttcactttggatgagccaacttctttcgctgccagaatcaacggtttgatctccattggtttgaacatcgatgaggaggaagagaaagagccagaacaggctactgaagctccaagtgaagaagctgttgctgagtctgccatggaggaggttgactagttgaatttaggtatatatagtgactgtgatatttagctaatgaaatctaattggatatttagaatgcctcatctcgtagcctatcaattactattaggccatctcttatgggcccttctttgaaattgcattcaaggggggatgggactattttgaatttgaagtttggactctgtgagctgtttggccaattgaagtcatccacttgtacacagggattcaccagtagtttagaacaattctctatcgttattctcttgtcgtctttggcaatacaagcgtcgatgactgagttggtgactttatgaagtctaagttgatatgagtttgaaattatgaaacagttttttacactggacatgtagatagggcccttgatgtttaggaagaggatacagtttgagatgttggagatgtgtgtggagggagcgaccacttttaaaaccacatgatccagacgttgctcagttatcgaagtttcggaaacaacctcagcttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatcaggcgcgccgaaacgatggctgttggctccaagtccttcgccactgcttccaaattgttcgacgccaagactaggagatccgtcttgatgctgtacgcctggtgccgtcattgtgacgacgtgattgacgaccagacacttggttttcaggccagacagcctgctttgcaaactcctgaacagcgattgatgcagctggaaatgaaaactagacaagcttacgccggtagtcaaatgcacgaaccagcattcgccgcatttcaggaagtcgccatggctcatgatatcgctcctgcctacgctttcgatcatttagaaggttttgctatggatgtgcgtgaggcccaatacagccagttggatgatactttaagatactgttatcatgtggctggtgtcgtgggacttatgatggctcagattatgggtgttagggacaatgccacactagatagagcatgtgatttgggtttagcttttcagttaactaatattgctcgtgatatcgtcgatgacgcccacgccggcagatgctatttacccgcatcatggcttgaacacgagggtttgaataaggagaactacgctgctcctgagaacaggcaagctttgtctagaattgccagaagattagttcaagaggccgagccgtactacttatccgcaactgctggtttagcaggcttgcctcttagatctgcctgggccattgcaactgccaagcaagtctacagaaaaattggcgtcaaggttgaacaagctggtcaacaagcttgggaccagcgccagagtaccaccacccctgaaaagttaacccttctgctcgctgctagtggtcaagccttaaccagtagaatgagagcacatccacctcggcctgcgcatctgtggcaacgtccattatgacctgcaggagacatgactgttcctcagttcaagttgggcacttacgagaagaccggtcttgctagattctaatcaagaggatgtcagaatgccatttgcctgagagatgcaggcttcatttttgattacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaggtccttaaaaaaggaacaggtaaggatatgtttttattgatgatggagatgtggtgcaagtgaatcctgagaacctcttttttcttttcaaacgcatttttgtcttcaattccattcttcgatcttttaacgatgggagcgcttattttgtctatgatgtggctttgaagatcagctgttgtattcaaactatcactttgagtcaacgagttcttaggtagtctttgaaaccgtgaaagggaacccattttcttcgaacccagggatttcactgatcctctggccattgacgccgatcgtgagttctgtagagttcccttcgtcttaagagagagggggaataattaaagatcaagtaatgttctacctacaaaagataaagatgaccttaatgtttttagcgaggtatagctgggagtcccaaagaagtagctagggcggtgagaggatttttttctcgtgcgcatataatcgctagcctagttaaagcatcttgacgacgtactaatatctggaagacttcagagcacagaaactatgcctggtgagttcatggtgaccgtattgagcacatccaaaaagatcttattctctccagtacaatcagcagaaggccttatccatcttgctgttccactacctcattccagtatacttctaatcatcgcctctagataagccagacgatctcaagaaccaccctcatcttgaaacgtggactcgagtcgcaatgtcctgtatcattcctacgtcacaagccatcactgggttctctcgcccccctacgaaacgctagctattgctatatggaacaatctagaccgtaagttagggccactctgttcatttctcgtcttagtcagctgatcctcgaaacgatctatccctttccttttccctatctttcttttcttttctttctcttgtatccgtgaaatatctcagtatccctgctacaactcaactacacacacaccaagcacaggcgcgccgaaacgatgactgtatgtgccaagaaacatgttcacttgactagagatgccgctgaacaactcttggccgatattgatagaaggttagaccagttgctccctgttgaaggtgaaagggatgtcgttggagcagctatgagagaaggagcactggcacctggtaagaggattaggcctatgcttctgttgcttacagctagagacttgggatgcgccgtttcccacgacggtctgcttgacctcgcttgcgcggtcgagatggttcatgcggcctctttaattctagacgacatgccttgtatggatgatgctaagctcagaagaggacgtcctacgatccacagccattacggtgaacacgtagcaattttggcagctgtagccctgctatctaaagcctttggtgttatcgcagatgcagatggattgacgccgctcgctaaaaaccgcgcagtaagcgagttgtccaatgccatcggaatgcaaggtcttgtgcaaggccaattcaaggacttgtctgagggtgataagccacgttcagctgaagctatcctgatgaccaatcactttaaaacatcaactctgttttgtgcttctatgcaaatggcttcaatcgtcgcaaatgcttcctccgaggctagagattgtttgcatcgattctctctggatttgggacaggcatttcaacttcttgatgacctcacagatggaatgacggataccggtaaagactcgaatcaagatgctggcaagtccaccctagttaacctattgggaccgcgtgctgtcgaggaacgactaagacagcacctacagcttgctagcgaacacctatctgcagcctgccaacatggtcatgccacgcagcactttattcaagcttggttcgataagaaattggctgccgtatcataacctgcaggagacatgactgttcctcagttcaagttgggcacttacgagaagaccggtcttgctagattctaatcaagaggatgtcagaatgccatttgcctgagagatgcaggcttcatttttgattacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaaccgttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatcaggcgcgccgaaacgatgaagcccaccactgttatcggagccggattcggcggactcgcactggccattagactgcaggctgccggtattccagtcctgctcttggaacaaagagacaaacctggaggtagagcttacgtttatgaggaccaaggttttaccttcgacgctggaccaactgtgatcactgacccttcagccatagaggagctgttcgctctcgccggaaagcaattaaaagaatatgtggagttactacctgtcaccccattctataggctttgttgggagtccggaaaggtctttaattacgacaacgatcaaacaagactggaggctcaaattcagcagttcaaccctagagatgttgaaggttacaggcaattcctcgattacagtcgcgctgtctttaaggagggatacttgaaattaggcactgttcctttcttatcgttcagagacatgctgagagccgctccacagctggccaagttgcaagcttggagatcagtttattcgaaagtggcttcatacattgaagatgaacatctaagacaagcatttagctttcattctctactcgttggtggtaatccttttgccacatcttcaatctacacattaatacatgctctggaaagggagtggggcgtttggttccctagaggtggaaccggtgcactggttcagggaatgattaaattatttcaggatctgggtggcgaagtggttttaaatgctcgtgtctctcacatggaaactacaggaaacaaaatcgaagctgtccacctggaagatggtcgtcgttttttgactcaagccgttgctagtaacgcagatgttgtgcacacttacagagatctcttgtctcaacatccggccgctgtaaagcaatccaataagctacagactaaaaggatgagtaactctctgttcgttttgtattttggcttgaaccaccaccacgatcaactcgcacatcacacagtctgttttggaccaagatatcgtgaattaattgatgaaattttcaaccatgatggactggctgaagatttttccttgtacttgcatgccccgtgtgttacggatagttcccttgcaccagaaggttgtggcagctactacgttctcgctccagttccacatttaggtactgcgaacttagattggaccgttgagggtccaaagctgcgagacaggattttcgcttaccttgaacaacactacatgcccggtctacgcagtcagttggtaacgcatagaatgttcactccatttgacttccgtgaccagcttaacgcctatcatggatctgctttctcagtagaacctgttttaacccaatctgcctggtttagaccacataatcgagacaaaaccatcacaaacctttacttggtgggtgctggtacccaccctggtgcaggaatacctggtgtgataggatcagccaaggccacggctggattgatgttggaggacctaatatgacctgcaggagacatgactgttcctcagttcaagttgggcacttacgagaagaccggtcttgctagattctaatcaagaggatgtcagaatgccatttgcctgagagatgcaggcttcatttttgattacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaggatccttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaatttttgacggctagctcagtcctaggtacgctagcattaaagaggagaaaatgactactcttgatgacacagcctacagatataggacatcagttccgggtgacgcagaggctatcgaagccttggacggttcattcactactgatacggtgtttagagtcaccgctacaggtgatggcttcaccttgagagaggttcctgtagacccacccttaacgaaagttttccctgatgacgaatcggatgacgagtctgatgctggtgaggacggtgaccctgattccagaacatttgtcgcatacggagatgatggtgacctggctggctttgttgtggtgtcctacagcggatggaatcgtagactcacagttgaggacatcgaagttgcacctgaacatcgtggtcacggtgttggtcgtgcactgatgggactggcaacagagtttgctagagaaagaggagccggacatttgtggttagaagtgaccaatgtcaacgctcctgctattcacgcatataggcgaatgggtttcactttgtgcggtcttgatactgctttgtatgacggaactgcttctgatggtgaacaagctctttacatgagtatgccatgtccatagcacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctgcggccgcaagaagttgattgagactttcaacgagattgctgaagacaaggaacaattcgagaagttttacagtgctttctccaagaacttgaagttgggtgtccatgaagacagccaaaacagatccgcattggccaagttgctgagatttaactccaccaagtctactgaggagctaacctcattctctgactacgtcaccagaatgccagagcaccagaagaacatctacttcattaccggtgagtctgtcaaggctcttgagaaatctccattcttggatgctttgaaggagaagaactttgaggtcctattgctgaccgatcctattgatgagtacgctatgactcaattgaaagagattgaggacaagaaattggttgacatcactaaagactttgagctggaagagtctgaggaggagaagaaggctagagaggaagaggttaaagatttcgagcctttgactaaagccctgaaagagattttgggtgacaaggttgagaaggttgtagtttcctacaagctggttgactctcctgctgctattagaacttcccaattcggctggtctgctaacatggaaagaattatgaaggctcaagctctgagagacaccaacaccatgtcctcgtacatggcttcaaagaagatcttcgagatctctccaaagtcgccaatcattaaggctttgagaaagaaggttgaggctaccggtacagaagagaccttaattaagcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaagacgtcccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggtgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataactgtcagaccaagtttactcatatatactttagattgatttaaaacttcatttttaatttaaaaggatctaggtgaagatcctt

SEQ ID NO: 2 - DNA, 3060 bp, Artificial Sequence
  Description of Artificial Sequence Synthetic polynucleotide
agcaacgcagaagcaagatgaaacacctgtttcttcatctataaatcctgctctagccagtttgctgtccaaacttaacggttaattcttaatgtcttccccaatcacttgagtacgaactatgtattatataatcaggcatcttttctttttttctttttttttttgtctctcgcttggatcccaacaccatatttcagatctcctgatgactgactcactgataataaaaatacggcttcagaatttctcaagactacactcactgtccgacttcaagtatgacatttcccttgctacctgcatacgcaagtgttgcagagtttgataattccttgagtttggtaggaaaagccgtgtttccctatgctgctgaccagctgcacaacctgatcaagttcactcaatcgactgagcttcaagttaatgtgcaagttgagtcatccgttacagaggaccaatttgaggagctgatcgacaacttgctcaagttgtacaataatggtatcaatgaagtgattttggacctagatttggcagaaagagttgtccaaaggatcccaggcgctagggttatctataggaccctggttgataaagttgcatccttgcccgctaatgctagtatcgctgtgcctttttcttctccactgggcgatttgaaaagtttcactaatggcggtagtagaactgtttatgctttttctgagaccgcaaagttggtagatgtgacttccactgttgcttctggtataatccccattattgatgctcggcaattgactactgaatacgaactttctgaagatgtcaaaaagttccctgtcagtgaaattttgttggcgtctttgactactgaccgccccgatggtctattcactactttggtggctgactcttctaattactcgttgggcctggtgtactcgtccaaaaagtctattccggaggctataaggacacaaactggagtctaccaatctcgtcgtcacggtttgtggtataaaggtgctacatctggagcaactcaaaagttgctgggtatcgaattggattgtgatggagactgcttgaaatttgtggttgaacaaacaggtgttggtttctgtcacttggaacgcacttcctgttttggccaatcaaagggtcttagagccatggaagccaccttgtgggatcgtaagagcaatgctccagaaggttcttataccaaacggttatttgacgacgaagttttgttgaacgctaaaattagggaggaagctgatgaacttgcagaagctaaatccaaggaagatatagcctgggaatgtgctgacttattttattttgcattagttagatgtgccaagtacggtgtgacgttggacgaggtggagagaaacctggatatgaagtccctaaaggtcactagaaggaaaggagatgccaagccaggatacaccaaggaacaacctaaagaagaatccaaacctaaagaagtcccttctgaaggtcgtattgaattgtgcaaaattgacgtttctaaggcctcctcacaagaaattgaagatgcccttcgtcgtcctatccagaaaacggaacagattatggaattagtcaaaccaattgtcgacaatgttcgtcaaaatggtgacaaagcccttttagaactaactgccaagtttgatggagtcgctttgaagacacctgtgttagaagctcctttcccagaggaacttatgcaattgccagataacgttaagagagccattgatctctctatagataacgtcaggaaattccatgaagctcaactaacggagacgttgcaagttgagacttgccctggtgtagtctgctctcgttttgcaagacctattgagaaagttggcctctatattcctggtggaaccgcaattctgccttccacttccctgatgctgggtgttcctgccaaagttgctggttgcaaagaaattgtttttgcatctccacctaagaaggatggtacccttaccccagaagtcatctacgttgcccacaaggttggtgctaagtgtatcgtgctagcaggaggcgcccaggcagtagctgctatggcttacggaacagaaactgttcctaagtgtgacaaaatatttggtccaggaaaccagttcgttactgctgccaagatgatggttcaaaatgacacatcagccctgtgtagtattgacatgcctgctgggccttctgaagttctagttattgctgataaatacgctgatccagatttcgttgcctcagaccttctgtctcaagctgaacatggtattgattcccaggtgattctgttggctgtcgatatgacagacaaggagcttgccagaattgaagatgctgttcacaaccaagctgtgcagttgccaagggttgaaattgtacgcaagtgtattgcacactctacaaccctatcggttgcaacctacgagcaggctttggaaatgtccaatcagtacgctcctgaacacttgatcctgcaaatcgagaatgcttcttcttatgttgatcaagtacaacacgctggatctgtgtttgttggtgcctactctccagagagttgtggagattactcctccggtaccaaccacactttgccaacgtacggatatgcccgtcaatacagcggagttaacactgcaaccttccagaagttcatcacttcacaagacgtaactcctgagggactgaaacatattggccaagcagtgatggatctggctgctgttgaaggtctagatgctcaccgcaatgctgttaaggttcgtatggagaaactgggacttatttaattatttagagattttaacttacatttagattcgatagatctaaccggcatctaacatccagtgtttctaatctagtgaatcgagatcaagtctcactctgaacataataaataaggtctaaccccatggaattacatgtccccttgggttctgagattgtcttacccggtggggaaagagccatactcaagttcgtaggaaatgtggagtcaaagtctgggatttttgctggtgttgaactgattgg

SEQ ID NO: 3 - DNA, 4421 bp, Artificial Sequence
  Description of Artificial Sequence Synthetic polynucleotide
gcgatcgcgtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgggcctcgatggcccagatctagggagggcatcattgaggtttccacaaaaggaagaaacatggatccagagacatcaacagagaggaaagcgggtagtgaagccgaagccacaacacagcccgatttggaagggagttcacaatcaaggtgagtccagccattttttttcttttttttttttttattcaggtgaacccacctaactatttttaactgggatccagtgagctcgctgggtgaaagccaaccatcttttgtttcggggaaccgtgctcgccccgtaaagttaatttttttttcccgcgcagctttaatctttcggcagagaaggcgttttcatcgtagcgtgggaacagaataatcagttcatgtgctatacaggcacatggcagcagtcactattttgctttttaaccttaaagtcgttcatcaatcattaactgaccaatcagattttttgcatttgccacttatctaaaaatacttttgtatctcgcagatacgttcagtggtttccaggacaacacccaaaaaaaggtatcaatgccactaggcagtcggttttatttttggtcacccacgcaaagaagcacccacctcttttaggttttaagttgtgggaacagtaacaccgcctagagcttcaggaaaaaccagtacctgtgaccgcaattcaccatgatgcagaatgttaatttaaacgagtgccaaatcaagatttcaacagacaaatcaatcgatccatagttacccattccagccttttcgtcgtcgagcctgcttcattcctgcctcaggtgcataactttgcatgaaaagtccagattagggcagattttgagtttaaaataggaaatataaacaaatataccgcgaaaaaggtttgtttatagcttttcgcctggtgccgtacggtataaatacatactctcctcccccccctggttctctttttcttttgttacttacattttaccgttccgtcactcgcttcactcaacaacaaaaggcgcgcctaagcgatggtctcaaggagagacgtgaaagtgaaacgtgatttcatgcgtcattttgaacattttgtaaatcttatttaataatgtgtgcggcaattcacatttaatttatgaatgttttcttaacatatcgcggcaactcaagaaacggcaggttcggatcttagctactagagattaaggaggtaaaaaaaatgagtgtgatcgctaaacaaatgacctacaaggtttatatgtcaggcacggtcaatggacactactttgaggtcgaaggtgatggaaaaggtaagccctacgagggggagcagacggtaaagctcactgtcaccaagggcggacctctgccatttgcttgggatattttatcaccacagtgtcagtacggaagcataccattcaccaagtaccctgaagatatccctgactatgtaaagcagtcattcccggagggctatacatgggagaggatcatgaactttgaagatggtgcagtgtgtactgtcagcaatgattccagcatccaaggcaactgtttcatctaccatgtcaagttctctggtttgaactttcctcccaatggacctgtcatgcagaagaaaacacagggctgggaacccaacactgagcgtctgtttgcacgagatggaatgctgctaggaaacaactttatggctctgaagttagaaggaggcggtcactatttgtgtgaatttaaaactacttacaaggcaaagaagcctgtgaagatgccagggtatcactatgttgaccgcaaactggatgtaaccaatcacaacaaggattacacttcggttgagcagtgtgaaatttccattgcacgcaaacctgtggtcgcctaaggatctctccaaagcccgccgaaaggcgggcttttctgtcgtctcaaggtacgtcttcatcgctatcctgcaggcggatatcttcggaaaaaaaaaggcctgcgattaccagcaggcctgttgagatcgaggaaatctcgggatctagtcttgagctttgctcactcaaaggcggtaatgcggccgctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcggtaccttgatcgggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatccctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcacgatgggcaaatattatacgcaaggcgacaaggtgctgatgccgctggcgattcaggttcatcatgccgtttgtgatggcttccatgtcggcagaatgcttaatgaattacaacagtactgtgatgagtggcagggcggggcgtaatttgatatcgagctcgcttggactcctgttgatagatccagtaatgacctcagaactccatctggatttgttcagaacgctcggttgccgccgggcgttttttattggtgagaatccaagcctcctcgaggtatcacgaggcagaatttcagataaaaaaaatccttagctttcgctaaggat

SEQ ID NO: 4 - DNA, 6930 bp, Artificial Sequence
  Description of Artificial Sequence Synthetic polynucleotide
gcgatcgcgtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgggcctcgatggcccagatctagggagggcatcattgaggtttccacaaaaggaagaaacatggatccagagacatcaacagagaggaaagcgggtagtgaagccgaagccacaacacagcccgatttggaagggagttcacaatcaaggtgagtccagccattttttttcttttttttttttttattcaggtgaacccacctaactatttttaactgggatccagtgagctcgctgggtgaaagccaaccatcttttgtttcggggaaccgtgctcgccccgtaaagttaatttttttttcccgcgcagctttaatctttcggcagagaaggcgttttcatcgtagcgtgggaacagaataatcagttcatgtgctatacaggcacatggcagcagtcactattttgctttttaaccttaaagtcgttcatcaatcattaactgaccaatcagattttttgcatttgccacttatctaaaaatacttttgtatctcgcagatacgttcagtggtttccaggacaacacccaaaaaaaggtatcaatgccactaggcagtcggttttatttttggtcacccacgcaaagaagcacccacctcttttaggttttaagttgtgggaacagtaacaccgcctagagcttcaggaaaaaccagtacctgtgaccgcaattcaccatgatgcagaatgttaatttaaacgagtgccaaatcaagatttcaacagacaaatcaatcgatccatagttacccattccagccttttcgtcgtcgagcctgcttcattcctgcctcaggtgcataactttgcatgaaaagtccagattagggcagattttgagtttaaaataggaaatataaacaaatataccgcgaaaaaggtttgtttatagcttttcgcctggtgccgtacggtataaatacatactctcctcccccccctggttctctttttcttttgttacttacattttaccgttccgtcactcgcttcactcaacaacaaaaggcgcgccgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagcaaggtatcgctcatcgcgaaagtgaaacgtgatttcatgcgtcattttgaacattttgtaaatcttatttaataatgtgtgcggcaattcacatttaatttatgaatgttttcttaacatatcgcggcaactcaagaaacggcaggttcggatcttagctactagagattaaggaggtaaaaaaaatgagtgtgatcgctaaacaaatgacctacaaggtttatatgtcaggcacggtcaatggacactactttgaggtcgaaggtgatggaaaaggtaagccctacgagggggagcagacggtaaagctcactgtcaccaagggcggacctctgccatttgcttgggatattttatcaccacagtgtcagtacggaagcataccattcaccaagtaccctgaagatatccctgactatgtaaagcagtcattcccggagggctatacatgggagaggatcatgaactttgaagatggtgcagtgtgtactgtcagcaatgattccagcatccaaggcaactgtttcatctaccatgtcaagttctctggtttgaactttcctcccaatggacctgtcatgcagaagaaaacacagggctgggaacccaacactgagcgtctctttgcacgagatggaatgctgctaggaaacaactttatggctctgaagttagaaggaggcggtcactatttgtgtgaatttaaaactacttacaaggcaaagaagcctgtgaagatgccagggtatcactatgttgaccgcaaactggatgtaaccaatcacaacaaggattacacttcggttgagcagtgtgaaatttccattgcacgcaaacctgtggtcgcctaaggatctctccaaagcccgccgaaaggcgggcttttctgtgcgatgttgctgacagaggtgactacaaagatgacgacgataaagattacaaagacgatgacgataaggactataaagatgatgacgacaaataataacctgcaggagacatgactgttcctcagttcaagttgggcacttacgagaagaccggtcttgctagattctaatcaagaggatgtcagaatgccatttgcctgagagatgcaggcttcatttttgattacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaggatccttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaatttttgacggctagctcagtcctaggtacgctagcattaaagaggagaaaatggctaaactgacctctgctgttccggttctgaccgctcgtgacgttgctggtgctgttgagttctggaccgaccgtctgggtttctctcgtgacttcgttgaagacgacttcgctggtgttgttcgtgacgacgttaccctgttcatctctgctgttcaggaccaggttgttccggacaacaccctggcttgggtttgggttcgtggtctggacgaactgtacgctgaatggtctgaagttgtttctaccaacttccgtgacgcttctggtccggctatgaccgaaatcggtgaacagccgtggggtcgtgagttcgctctgcgtgacccggctggtaactgcgttcacttcgttgctgaagaacaggactaacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctgcggccgcagatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacttaattaagcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaagacgtcccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggtgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataactgtcagaccaagtttactcatatatactttagattgatttaaaacttcatttttaatttaaaaggatctaggtgaagatcctt

SEQ ID NO: 5 - DNA, 7893 bp, Artificial Sequence
  Description of Artificial Sequence Synthetic polynucleotide
gcgatcgcggtctcaccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattagaatggtatatccttgaaatatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcgctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtagatatactagaagttctcctcgaggcctcgatggccgcaatgtatgactttaagatttgtgagcaggaagaaaagggagaatcttctaacgataaacccttgaaaaactgggtagactacgctatgttgagttgctacgcaggctgcacaattacacgagaatgctcccgcctaggatttaaggctaagggacgtgcaatgcagacgacagatctaaatgaccgtgtcggtgaagtgttcgccaaacttttcggttaacacatgcagtgatgcacgcgcgatggtgctaagttacatatatatatatatatatatatatatatatatatagccatagtgatgtctaagtaacctttatggtatatttcttaatgtggaaagatactagcgcgcgcacccacacacaagcttcgtcttttcttgaagaaaagaggaagctcgctaaatgggattccactttccgttccctgccagctgatggaaaaaggttagtggaacgatgaagaataaaaagagagatccactgaggtgaaatttcagctgacagcgagtttcatgatcgtgatgaacaatggtaacgagttgtggctgttgccagggagggtggttctcaacttttaatgtatggccaaatcgctacttgggtttgttatataacaaagaagaaataatgaactgattctcttcctccttcttgtcctttcttaattctgttgtaattaccttcctttgtaattttttttgtaattattcttcttaataatccaaacaaacacacatatggcgcgccgaaacgatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttaacttatggtgttcaatgtttttctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagatcacatggtcttgttagaatttgttactgctgctggtattacccatggtatggatgaattgtacaaataaggtacgtcttcatcgctatcctgcaggagacatgactgttcctcagttcaagttgggcacttacgagaagaccggtcttgctagattctaatcaagaggatgtcagaatgccatttgcctgagagatgcaggcttcatttttgattacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaggatccttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaatttttgacggctagctcagtcctaggtacgctagcattaaagaggagaaaatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatcccagggaaaacagcattccaggtattagaagaatatcctgattcaggtgaaaatattgttgatgcgctggcagtgttcctgcgccggttgcattcgattcctgtttgtaattgtccttttaacagtgatcgcgtatttcgtcttgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctgcggccgctcgactacgtcgttaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatataaattaggaatcatagtttcatgattttctgttacacctaactttttgtgtggtgccctcctccttgtcaatattaatgttaaagtgcaattctttttccttatcacgttgagccattagtatcaatttgcttacctgtattcctttacatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggttgaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtaggaaattcgttcttgatggtttcctccacagtttttctccataatcttgaagaggccaaaacattagctttatccaaggaccaaataggcaatggtggctcatgttgtagggccatagagaccttaattaagcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaagacgtcccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggtgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataactgtcagaccaagtttactcatatatactttagattgatttaaaacttcatttttaatttaaaaggatctaggtgaagatcctt

SEQ ID NO: 6 - DNA, 7715 bp, Artificial Sequence
  Description of Artificial Sequence Synthetic polynucleotide
gcgatcgcatttaaatggacgagtccggaatcgaaccggagacctctcccatgctaagggagcgcgctaccgactacgccacacgcccgaacgtttgttgaaatcttccaaacagaaatgatacaggtttactttacagcaagacattagtagtttcattagcttatcaacatgtatgaaaccggcaagattgaaagcttgattgcccttgacgagctactcaaatgtgtaaatatctgcacaatcagtttacactcatgtttccgcgcttttaccctccatctagattaaaatcttatatggattatttgggataaatagcatttagtaaatcaaattgataaaaaaaagaatagcatttaggtatgcagaatatactagaagctgtcctcactgatcctgaatccaaaaaagagaattctacgtaataatattatcacctcttcctccattttatatgctgtcattcattatcctattccattaccaatccttgcatttcagcctccattaaatccgatggttgtttctcaacttttatgccatcttcttatacccaggcctcgatggcccagatctagggagggcatcattgaggtttccacaaaaggaagaaacatggatccagagacatcaacagagaggaaagcgggtagtgaagccgaagccacaacacagcccgatttggaagggagttcacaatcaaggtgagtccagccattttttttcttttttttttttttattcaggtgaacccacctaactatttttaactgggatccagtgagctcgctgggtgaaagccaaccatcttttgtttcggggaaccgtgctcgccccgtaaagttaatttttttttcccgcgcagctttaatctttcggcagagaaggcgttttcatcgtagcgtgggaacagaataatcagttcatgtgctatacaggcacatggcagcagtcactattttgctttttaaccttaaagtcgttcatcaatcattaactgaccaatcagattttttgcatttgccacttatctaaaaatacttttgtatctcgcagatacgttcagtggtttccaggacaacacccaaaaaaaggtatcaatgccactaggcagtcggttttatttttggtcacccacgcaaagaagcacccacctcttttaggttttaagttgtgggaacagtaacaccgcctagagcttcaggaaaaaccagtacctgtgaccgcaattcaccatgatgcagaatgttaatttaaacgagtgccaaatcaagatttcaacagacaaatcaatcgatccatagttacccattccagccttttcgtcgtcgagcctgcttcattcctgcctcaggtgcataactttgcatgaaaagtccagattagggcagattttgagtttaaaataggaaatataaacaaatataccgcgaaaaaggtttgtttatagcttttcgcctggtgccgtacggtataaatacatactctcctcccccccctggttctctttttcttttgttacttacattttaccgttccgtcactcgcttcactcaacaacaaaaggcgcgccgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagcaaggtatcgctcatcgcgaaagtgaaacgtgatttcatgcgtcattttgaacattttgtaaatcttatttaataatgtgtgcggcaattcacatttaatttatgaatgttttcttaacatatcgcggcaactcaagaaacggcaggttcggatcttagctactagagattaaggaggtaaaaaaaatgagtgtgatcgctaaacaaatgacctacaaggtttatatgtcaggcacggtcaatggacactactttgaggtcgaaggtgatggaaaaggtaagccctacgagggggagcagacggtaaagctcactgtcaccaagggcggacctctgccatttgcttgggatattttatcaccacagtgtcagtacggaagcataccattcaccaagtaccctgaagatatccctgactatgtaaagcagtcattcccggagggctatacatgggagaggatcatgaactttgaagatggtgcagtgtgtactgtcagcaatgattccagcatccaaggcaactgtttcatctaccatgtcaagttctctggtttgaactttcctcccaatggacctgtcatgcagaagaaaacacagggctgggaacccaacactgagcgtctctttgcacgagatggaatgctgctaggaaacaactttatggctctgaagttagaaggaggcggtcactatttgtgtgaatttaaaactacttacaaggcaaagaagcctgtgaagatgccagggtatcactatgttgaccgcaaactggatgtaaccaatcacaacaaggattacacttcggttgagcagtgtgaaatttccattgcacgcaaacctgtggtcgcctaaggatctctccaaagcccgccgaaaggcgggcttttctgtgcgatgttgctgacagaggtgactacaaagatgacgacgataaagattacaaagacgatgacgataaggactataaagatgatgacgacaaataataacctgcaggagacatgactgttcctcagttcaagttgggcacttacgagaagaccggtcttgctagattctaatcaagaggatgtcagaatgccatttgcctgagagatgcaggcttcatttttgattacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaggatccttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaatttttgacggctagctcagtcctaggtacgctagcattaaagaggagaaaatggctaaactgacctctgctgttccggttctgaccgctcgtgacgttgctggtgctgttgagttctggaccgaccgtctgggtttctctcgtgacttcgttgaagacgacttcgctggtgttgttcgtgacgacgttaccctgttcatctctgctgttcaggaccaggttgttccggacaacaccctggcttgggtttgggttcgtggtctggacgaactgtacgctgaatggtctgaagttgtttctaccaacttccgtgacgcttctggtccggctatgaccgaaatcggtgaacagccgtggggtcgtgagttcgctctgcgtgacccggctggtaactgcgttcacttcgttgctgaagaacaggactaacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctgcggccgcttatatattggcccaaaagggatcattaacgaattgcgaaatgggtaatctctttacagttaacagattctctggtgattggctttcctcaaggtagcaaatatactctaattgtagcacgtctactatgtatttctttaccttgtcatagcacacacccacttgcgccacgccgtgtgcgtcatcacttagaacaaatctggatccacagtgcttcttgaccagattacataaggttttgctggggtacggctcctcgaggcgctttcttaatgcggacgtattgatttcaattgcgccgccataggagtctataaattgtaaatttcttacaactgcatcgtatatttctggccattcactgatgacgtccagtgaagctacaggaactccggtttcttcgttgcagttgcccgatttctggtttactagcatgtcattgggcaaaaataatttgtaaaggtcgaagtgacccacgaccaacggtttaatattgatcagcatttcgtactgtgattggaagtaagacaggagaaaatgtttcaaattatcattgaaggaatgcaatgaattgtaccattgttgttggtcgaaatcaatagggatcccgttgacgtgatggaccgaacccacacaaaacttcaaaatatcattattctccttcatgagtcgctttgcatattcgatatgagccatgtcacaactttcgatctccattcctataatgaatttagtccgcacatcgggtctatcagcataacgagtcttgatttcttgcgcatgactcatgaaattcttgaacgatgtttctagcttggttatgacttcctcaggattcttgcccaatgactgctcttcggggtatataaacttggcctcaattcttggtatgtgctctgtcaaacagtacgtgtgaaagttgaggttgaccacttgatcgaccacggaatccaaagggtccgtaccgtgggcactatagtcaccggagtgtgaatggtgtgagtgcatgacctcgaggatttttactgaattgtacgtatcaagaagtactaaaatgtgtttgaaagggaaagattgtacgggaaatggaaaatgtacatataatatccagtatcatttgagttgatagagattatctctatagatagagagatttgacaaaaaaaataaaaaaatttaaatttaattaagcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaagacgtcccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggtgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataactgtcagaccaagtttactcatatatactttagattgatttaaaacttcatttttaatttaaaaggatctaggtgaagatcctt

SEQ ID NO: 7 - DNA, 40 bp, Artificial Sequence
  Description of Artificial Sequence Synthetic primer
ctaagaggcctcgatggccttggaggaattgaagcctgac

SEQ ID NO: 8 - DNA, 39 bp, Artificial Sequence
  Description of Artificial Sequence Synthetic primer
ctaagaggcctcgatggcccaagggaaaacgggtggttg

SEQ ID NO: 9 - DNA, 41 bp, Artificial Sequence
  Description of Artificial Sequence Synthetic primer
ctaagaggcctcgatggccgattgctccagaaaaatgttgg

SEQ ID NO: 10 - DNA, 41 bp, Artificial Sequence
  Description of Artificial Sequence Synthetic primer
ctaagaggcctcgatggcccgatgtgttgtgggtcttgcag

SEQ ID NO: 11 - DNA, 46 bp, Artificial Sequence
  Description of Artificial Sequence Synthetic primer
ctaagaggcctcgatggccgtagtttgacacatagtgcatttattg

SEQ ID NO: 12 - DNA, 41 bp, Artificial Sequence
  Description of Artificial Sequence Synthetic primer
ctaagaggcctcgatggccactaataatgaaacgcccaaac

SEQ ID NO: 13 - DNA, 38 bp, Artificial Sequence
  Description of Artificial Sequence Synthetic primer
ctaagaggcctcgatggccttactactagtatacatag

SEQ ID NO: 14 - DNA, 39 bp, Artificial Sequence
  Description of Artificial Sequence Synthetic primer
ctaagaggcctcgatggcctctaataatctgaaaactcg

SEQ ID NO: 15 - DNA, 38 bp, Artificial Sequence
  Description of Artificial Sequence Synthetic primer
ctaagaggcctcgatggccctagacttgatgttcaacc

SEQ ID NO: 16 - DNA, 39 bp, Artificial Sequence
  Description of Artificial Sequence Synthetic primer
ctaagaggcctcgatggccatctttcactagatactcgg

SEQ ID NO: 17 - DNA, 40 bp, Artificial Sequence
  Description of Artificial Sequence Synthetic primer
ctaagaggcctcgatggccttttttgtagaaatgtcttgg

SEQ ID NO: 18 - DNA, 35 bp, Artificial Sequence
  Description of Artificial Sequence Synthetic primer
ttcagtggcgcgccagtgtaggtttgtatgtgtac

SEQ ID NO: 19 - DNA, 35 bp, Artificial Sequence
  Description of Artificial Sequence Synthetic primer
ttcagtggcgcgccttgtaatggttgaaggactac

SEQ ID NO: 20 - DNA, 42 bp, Artificial Sequence
  Description of Artificial Sequence Synthetic primer
ttcagtggcgcgcccgttaaacaaaaaaaattgttttcaatg

SEQ ID NO: 21 - DNA, 37 bp, Artificial Sequence
  Description of Artificial Sequence Synthetic primer
ttcagtggcgcgccaattggtgatgaagcttcttcag

SEQ ID NO: 22 - DNA, 37 bp, Artificial Sequence
  Description of Artificial Sequence Synthetic primer
ttcagtggcgcgccattgtaattgtaatgtataggtg

SEQ ID NO: 23 - DNA, 41 bp, Artificial Sequence
  Description of Artificial Sequence Synthetic primer
ttcagtggcgcgcccttgtagtcttattggggggtataaag

SEQ ID NO: 24 - DNA, 33 bp, Artificial Sequence
  Description of Artificial Sequence Synthetic primer
ttcagtggcgcgcctttgatttgtttaggtaac

SEQ ID NO: 25 - DNA, 35 bp, Artificial Sequence
  Description of Artificial Sequence Synthetic primer
ttcagtggcgcgccctttgcaacagagaagtaatc

SEQ ID NO: 26 - DNA, 36 bp, Artificial Sequence
  Description of Artificial Sequence Synthetic primer
ttcagtggcgcgccgattaattattgagatagatag

SEQ ID NO: 27 - DNA, 34 bp, Artificial Sequence
  Description of Artificial Sequence Synthetic primer
ttcagtggcgcgccgagtaactttgcgtggagtg

SEQ ID NO: 28 - DNA, 26 bp, Artificial Sequence
  Description of Artificial Sequence Synthetic primer
ttcagtggcgcgcctgatagttgttc

SEQ ID NO: 29 - DNA, 1021 bp, Artificial Sequence
  Description of Artificial Sequence Synthetic polynucleotide
ggcctcgatggccttggaggaattgaagcctgacgctactatcacgatcaaagatggcgatcttcgtagtttgatccggggaaagtccaaccctcaaaaactttttatgagtgggaagctgaaaatcaagggaaatgtggctaaagccgcttcaatcgaaacagtgttgaagagcactaggccagccaagtccaaactatgagattcttatttacaaaaaaatgaataaattaactaactattttcagtaagtgtctcccttcgaactcaagaattgatactacccttatacctgatcttaaaatttttaacctttttctcctcacgtggattctttccccaaaggaggatccccacggagattctgttcactagcgtcttttgctgaaccccggtgtgtactgcgaaatggcctcaccgaattctgtggtgcccaagcttctccgataagtgggtgttctagcgactgctgcatcataggccctacttgagaataaaaagaaccgagaaaaagaaacgcgcaaacacagtgtggttgcgccaaatccgaagagactcacggaagtttccacgatcctctaaaatagatatcccttacgttgcactatccttgttatcgactcccctggatagttatcaatttcatcaataccccagattgcatcttcttaacggacaagcatatatcggctcttgaaaaatggaccgtcaacggaccggtatcggctctcgtaaaaatccggcccaatcaggtggatgcggctctcctatcaccatgtaatggcgtgcggctgaacatgggtggaaaagaaaccaattgccattggccaatcacaagcggaaacacctcctatcggctgcatacctgtgcggtgcccctattgtcttctcactcacttcctctctatggccagtgataaagttctgaaaaatatgtttaaggaggtcaaaaagtatttaaagagttctggccccagtagctaaagtcttcagtacacatacaaacctacactggcgcgcc

SEQ ID NO: 30 - DNA, 1021 bp, Artificial Sequence
  Description of Artificial Sequence Synthetic polynucleotide
ggcctcgatggcccaagggaaaacgggtggttgtggatcttgtgctttgggggatgccttccgatgcgatgggtgtccgtacttaggactaccacccttcaaaccgggccaggctatatcaattgagggactaggagcggatatttaaaggactattggttataatattattatttatttaaaaggaggtgatttattgtagacatccgaaatcgctctagccggtcgacttgccagagccaatgatttgtgtgtgctcaccattttagagctaaagaggtcactaggccaaagctaggggtggctgcacactgactacccttcatggggtgtacagcaccccctcttgtgcttggttcccttaaccgtcagattggcttacttttgacccaataagcagctactgaaacggaaatagtaacttctcgacctccgataggtccatacccgctcaaacaatttggcccttgttttacgtcccatctggggtaattagctcctgtgatgttctgtgtgatgtcagttagatttcggtaatcgtaagaccgactcagctctagtgagcgcccgcgatgtccaataaatgcgtaatctcgcgcagcttctgcgctggcccacacggaaatcaccccctcaaccccctagtttccctcccctctccttgtgaacaagaggccaaccgcaactttggactggttgcggaatttccgaagtccgggtaaactatcctctgtgtcttcctttcaacaagattggttgaacagagactaactagctacaatcatctcctaattggctacctgggggctctccaaccaacacagggcgtccgcgccccaaaaattgcagcattcagaaactttttttctttgaaaaagaaattacgcaagaattttgaaatctctttcaaacttatcgtgtctggcgagaagggctccactgaactaattttcctcgttctcttttccctaacccaatctcacctgtgatttgtagtccttcaaccattacaaggcgcgcc

SEQ ID NO: 31 - DNA, 1021 bp, Artificial Sequence
  Description of Artificial Sequence Synthetic polynucleotide
ggcctcgatggccgattgctccagaaaaatgttggataaagggttggtgattatggcaggaagctggcctatgttggaggacttcttcctctttgactcatacttcttgttctcttgttggttactcctctgagtctccgagtcgttcgactctgaaacgtctttgagcattccctggatgaatattcagtatggatagttggcaattgtttatctgtaacctgcgcggctggagatggtatgtagtacgtagtgcattgctggaaggggcgcgcaaacgagctagttgggagtatgccaagattgacctccctggacggggaatgcgtggtacaactctaaaatatttataagccaatactggggtatcagtgtagtaaaatcaagaaagtcactggcaagaaaggtcaagatccacccgctactgtttcgaaaaatctcctggaggtttttgaagtttttcatcgaatttttcaataaccctttagggctgagaaccaggaccactgcacatggggtaaaactaatcctggaaacttgctcaaatctcctattagataggaaattgaactaaatctcaaacactattcccacggtgggctttcttctgtttactagcggatttctggcatctcggtaatgctcttacatccttgcatctaggaatgaagttcttggttttatttatcaaaaaaaaattagtagtacttgacaaactcctgccttgactactcgtaaatgcagctaccgtcagaattccatctaaatcaacttatgtcccgttacgacgaaattggtaaatttcatcgcactcactctacaccatttgtaggatgccagatgttcgctaacatctcatctccgcgtcggattcgattctccgcgttcccgcttctacacgacttcttcccggggtaaccctcacagcttccattgaacctcccttcatataagaagcgtcatttatccgccgacgacacttcctcattgaaaacaattttttttgtttaacgggcgcgcc

SEQ ID NO: 32 - DNA, 975 bp, Artificial Sequence
  Description of Artificial Sequence Synthetic polynucleotide
ggcctcgatggcccgatgtgttgtgggtcttgcagattagatctgatggaatagggtgttgcattgtaacaaaacaaaaggatacagtgcaggaatggatgacttggcgggattcttgactctgttattgacataaagggattaaagtctaaatgggaacattgggtagtatcttagccaggcaagaattgtgtctgattttgaccctgcaaaaatgatattcgcgcaattcggtaactttagtttgacttctcgatgtgacttctctctgactcgttaactctaatcttcggagtcaccccccaaagcttaaaaacttacacctgttgcacaatgcctgtcatttagactttattttccctatctaatctatctgttactacatgcccaccatagaatcaccaaaataacgactattccttactccctttacaaacaggatcattctgaaagatgagtaatacagcgagagccaatgggatttcctggaagtgaatgggcgcatatttttcagccgaaaactgggttctgattgggcaatagataagtatttggtagacacacgacaagctccactgaggggaccgtagttttccagtcgtgtatggatcaaaataagataaagagatgccagaagttcagcataaatcaagtgaaaatttcatgaatcgatagcaattgagaggataaaaccggctaatcctatctcttgtctgaaaaagatgcctccatttgccagttgaagtttgaaacatgttttgaaatcacttaatcgtaaatagctacacttgttcctaggtcccagtatgactgaacttttccaattatgagtaaagccgcgagccttatccaaaaaagaaaagttgagcatatataaggcactgtccacgaacactatttgtctggaaacctatctcttcctcttcctttccagctaagaaatactgaagaagcttcatcaccaattggcgcgcc

SEQ ID NO: 33 - DNA, 941 bp, Artificial Sequence
  Description of Artificial Sequence Synthetic polynucleotide
ggcctcgatggccgtagtttgacacatagtgcatttattggaaatatggggacactaccttccgccaataggagacgagaatacgactagacgtatctcgtaaagtccctccgcctcgatctagaactgccaccatatttgaggatcaaagaccagaagaagtgtcgtgccagtttttcagataaatcattcgccgagatcagtcgatcatcagcgttcagtccatgagaacaaggtgctggcttgacgtatcttcaggcagccagcagctttttgtttcagcctatcatctttgaggcttatcatcttactgataagactatgggaaaaaacccaagaaaatgagatgttttatgtgaacgaccatttacttttttagataggcaatcttctgccccggtgagtctcctcattgggcaatgttgtaattacacgactaacaaatcagaggcggtaaacgctagtctcggcagcgtcatcgaggtcagtgcatactcaacaatagaaggcgcgcaatcattagacaacttgatccatagcccgcagcgtttcttctccagaaagttcactataattacccgtagaggattggttcaacatacaagaagatcagcaaatcaccttcagtattcggcgacaagatttatttttcactgaggtttcaaagcttttcaaatttcaatataatacttccctattaatcatgcatgcaccctacaagatttcgcattcttttttttactacaggaattactgaccaggtgactctaaaatagctttgaggtatataaatcacaaaggcgctgttagcagacagtttttttctttttcgtttcttaccttcggtcaacaatattaattcgcttcaagctacttagttctctccattataagagaatcttaatataaacacctatacattacaattacaatggcgcgcc

SEQ ID NO: 34 - DNA, 1021 bp, Artificial Sequence
  Description of Artificial Sequence Synthetic polynucleotide
ggcctcgatggccactaataatgaaacgcccaaactctgtagatatttagcttgtctaaaaatctctatagcgtctgttgcagtcttaccctgcactctgtaacctcctagggcgttgaacttttgtggttgtagtccaacgtgaccagtcactggtattcctatatctataagcttcttcacttgatctgttatttcataagagccctccagctttatgctttggacttttcccagcttcatcaactttatggcatttcttccgcattcttctgcagatgtttcaaaagttccaaagggcagatctgcaattataaattttctgtcaatggctcgacaagcagctttgcaacagtaatagaactcttcaaaagacatttccaaagtcgactggtagcccaaattgatcatggaaagagaatctcctatgattacggcatctgcctcgctctcattagcgtattttccacttatataatcgtgagctgtgatgacacttataggcttacgatcctgatactttgagtataatgttctcaaagtggactgcctggaaaccttggccttaggatatatgggcacttgagagtaatttcgtatagctcgcttaaataatagtagcatagttaattttcaagcagttgagagaaaaaaatgcgagagatgcaccgttatttacccgaatatagtttgttcgcggacatctctcatgctaatcttgctccaaagatcaaagtcccgagaactcggctcagattttcccatgtgctttcggtaagatggtttactgccaactttaagtttgcccactatcgattattggaataaagtttttgagattattctgattcgaatcgacagggaaaggaatacgttgactcactcagtaccttaaactctgtaagcgccgtagacatactagcaagtacactactagacctagccacagcacgaaccaagaggcgagcctctcagctctgttcaaactttataccccccaataagactacaagggcgcgcc

SEQ ID NO: 35 - DNA, 1021 bp, Artificial Sequence
  Description of Artificial Sequence Synthetic polynucleotide
ggcctcgatggccttactactagtatacatagaataaaaacggtaatagaactgggaactaagcagaaacttacaattcctgagaagccttggccttggcagacttctttggcaacaattcggattgaatgtttggcaagacaccaccttgggcgatggtgacgtgtcccagcaacttgttcaattcctcatcgtttctgatggccaattgcaagtgtcttgggataattctggacttcttgttgtctctggcggcgttaccggccaattccaaaatttcagcagccaagtactccaagacagcagtcaaatagactggagcaccagaaccaattctttgggcgtagttacctcttctcagaagacggtggactcttcccacagggaaggtcaaacctgccttagaagatcttgaggttgaggccttttcagccgaagatgcttttcctttaccaccggacattgttgtagttttaatatagtttgagtatgagatggaactcagaacgaaggaattatcaccagtttatatattctgaggaaagggtgtgtcctaaattggacagtcacgatggcaataaacgctcagccaatcagaatgcaggagccataaattgttgtattattgctgcaagatttatgtgggttcacattccactgaatggttttcactgtagaattggtgtcctagttgttatgtttcgagatgttttcaagaaaaactaaaatgcacaaactgaccaataatgtgccgtcgcgcttggtacaaacgtcaggattgccaccacttttttcgcactctggtacaaaagttcgcacttcccactcgtatgtaacgaaaaacagagcagtctatccagaacgagacaaattagcgcgtactgtcccattccataaggtatcataggaaacgagagtcctccccccatcacgtatatataaacacactgatatcccacatccgcttgtcaccaaactaatacatccagttcaagttacctaaacaaatcaaaggcgcgcc

SEQ ID NO: 36 - DNA, 1021 bp, Artificial Sequence
  Description of Artificial Sequence Synthetic polynucleotide
ggcctcgatggcctctaataatctgaaaactcgatttgttaatattggacgagacaccctttttgaactagaaaaaggccaggaagtccccatgaacgtaagggttaccgttgatttgaaaaagaaaatagttgtatctcccttggatgcttacggaatcacaggtttaaaatcaaacttcggctacagtgtcagagctgtcaaggaatttcaccaaatatatacagagagcggtaatccagatggttactctcgatcctgtttcgttgaagcaggggattttttcataaaccaatctcacacgcaggatcacaatatggaaaagttaccaatgattgataattccgaggaatttaaagatggtcaagagcatcttttactagtgatcacaagatggagagagctccaagaatttttcaaagctgacaattccgacatgctcaaagagatagagagctgcgagtcaatgtttgataaccgtcttgagataatcaacggaaccaaactagaagatgcgattctgatagcccttgccaaactagagagttagtatgttgatagcatcaaagttctaggatgtcagatgtctagaatcgttctgattcgaattgttcattttgaggcatatccaaaccattttgggcttgtttggatgcaagtttcttcgcgcgtgtattgctccctacgttatcaccacgacaactaaccgtctagatccgaaacagtgagtccttcaattggaagttcgtctacaggtgacgggaaaagaaccataaaatcatagtaaataaatgaaatcagtatcttaattatccctactaacccatccttgttgctaggtatgctctcgtatagtgtctcctcaaaaactcaccgaagctaaaaatagaaccgtatgatgtggtcgttccccacccgacaatctcgatatttcaaacccatctcccgccctttccttttcagtgttttcttttagattagcttcttctactgattacttctctgttgcaaagggcgcgcc

SEQ ID NO: 37 - DNA, 1021 bp, Artificial Sequence
  Description of Artificial Sequence Synthetic polynucleotide
ggcctcgatggccctagacttgatgttcaacctgacaaaaattgctgccgagggattgatcattttcctttgtggatatttgatatcagtactgtattgccagataatataatgtgtaccattttcaagagtgctttgcttcactatatttgttgtatctagatgggccgaagctataacatcagggtcaatatcatcgtctccattatgagcagataaagtgattttgaaatcaattgaagtgtactgttggaccatatcctgaaattcctcttgactcactacatttgatttcaatacgatgtaagttttcaatgtatcactgaagaaggcaaaagtcttattctctgtttggcacaacagatcataaaaatcaggttcattttctttagattccagatttcccgaaggaatatggagttttagatccatatgttgatcaataaatggttagctgtatctcttcggaccctctaaagaaacgcgttcactgttgacccgttagataactctgttcgcgcttcaccgttgcagttgagcagcctaagaacacctgatcagcaggagtactatccaaattacattgaggctacctagcaagtggtatcccaattaacataccagatatcggtataaacatcaaaagtgtgtgacaaggtgcaaagaatttggtccattttagagtcaatgggtctgggtactagttagcttgcgctcttcccaatattcgcagggtttaataataagcaactttttaggttatctacgaagttcatctctcaccatggtcaactacagccattggctcaaccagcaccgatggtaatagttaacgcagcttcaatgtctagtgagccaaaatatgcttaggagattaagttccggtcacgatgttcccggaattcattatttccgacctagccccgaaccttctctgatgttctccaggaatcgctctcaccacatcgctttgccatcctgactattaatcctatctatctcaataattaatcggcgcgcc

SEQ ID NO: 38 - DNA, 1021 bp, Artificial Sequence
  Description of Artificial Sequence Synthetic polynucleotide
ggcctcgatggccatctttcactagatactcggcattttgaggggaaattttgcttggaaaatagggatatttcaacgatatgttgctattcaaccattgggctgccgtatttcctccccaatctaatcttctggcgtaagttaaaacagcacttccatcgactataggtataacatttgtagactcatgacccgttccaataacaagaccatttggagaatcggtattgtaatggtaggcaaataccgcatctacaccgctaacaactttgggaacattgtaagcttcaaacaagagctgatacattccagctctttgcatgtacggtgttgcaagaagttcattgatgataatcggattctcaactccatgtggagatgacactccaatatgagaaaaaccgtaatccaatagtgtttccacagaatcccaattggtgattaattgtccatcaaatggagactttacgttcgacttgttgttgcttgcctccatgtaaacatcattgcctgccaatataagcatcttgttcctctttctgtcacgactcttggataccacagtagggaaaacggatgttggagaatccttgttcaccagccctatcctcatactagtttttccaaagtctatagctattggaactcctggggtgtaatctgaatgaaaaggcgccacatgtgaggaaaccggaatatcccttagaggatagaccttctgaggaggcaactccttctctgaaccaggcatttacgtgatacggcaggaaataggatggtgggaagctgaagatcgagcgagggcacaggagcagataataagggtgacattctggaaaggaaagctcgaaggcccgcctagaaacactgcgaggtaagtgggatcgaagcgcatcaaaaagaaaaaacggtagcaggagtgggattgccttcttaccgggcacgtcgcggcacgcttaatacaacacattctaagccgttaactgcccctttcactccacgcaaagttactcggcgcgcc

SEQ ID NO: 39 - DNA, 501 bp, Artificial Sequence
  Description of Artificial Sequence Synthetic polynucleotide
ggcctcgatggccttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatcaggcgcgcc

SEQ ID NO: 40 - DNA, 40 bp, Artificial Sequence
  Description of Artificial Sequence Synthetic primer
ctaagaggcgcgccgaaacgatggctacaaaaagagacca

SEQ ID NO: 41 - DNA, 40 bp, Artificial Sequence
  Description of Artificial Sequence Synthetic primer
ctaagaggcgcgccgaaacgatgcctcctaaacatcggct

SEQ ID NO: 42 - DNA, 40 bp, Artificial Sequence
  Description of Artificial Sequence Synthetic primer
ctaagaggcgcgccgaaacgatgacatctccctccccacc

SEQ ID NO: 43 - DNA, 40 bp, Artificial Sequence
  Description of Artificial Sequence Synthetic primer
ctaagaggcgcgccgaaacgatggcagaaaaactgatcgg

SEQ ID NO: 44 - DNA, 40 bp, Artificial Sequence
  Description of Artificial Sequence Synthetic primer
ctaagaggcgcgccgaaacgatgaaaatagtttttaaaga

SEQ ID NO: 45 - DNA, 40 bp, Artificial Sequence
  Description of Artificial Sequence Synthetic primer
ctaagaggcgcgccgaaacgatgccggggtgcaatataat

SEQ ID NO: 46 - DNA, 40 bp, Artificial Sequence
  Description of Artificial Sequence Synthetic primer
ctaagaggcgcgccgaaacgatgaacgagaaggacaaggt

SEQ ID NO: 47 - DNA, 40 bp, Artificial Sequence
  Description of Artificial Sequence Synthetic primer
ctaagaggcgcgccgaaacgatgtcgcttgaacatctcgg

SEQ ID NO: 48 - DNA, 40 bp, Artificial Sequence
  Description of Artificial Sequence Synthetic primer
ctaagaggcgcgccgaaacgatgacggagtcaatagtcgc

SEQ ID NO: 49 - DNA, 40 bp, Artificial Sequence
  Description of Artificial Sequence Synthetic primer
ctaagaggcgcgccgaaacgatgtcagagactgaaaacgc

SEQ ID NO: 50 - DNA, 40 bp, Artificial Sequence
  Description of Artificial Sequence Synthetic primer
ctaagaggcgcgccgaaacgatggcccaaggtttccctgt

SEQ ID NO: 51 - DNA, 40 bp, Artificial Sequence
  Description of Artificial Sequence Synthetic primer
ctaagaggcgcgccgaaacgatgacgcccaaaccgtcatt

SEQ ID NO: 52 - DNA, 40 bp, Artificial Sequence
  Description of Artificial Sequence Synthetic primer
ctaagaggcgcgccgaaacgatgtccaaagagaccaactt

SEQ ID NO: 53 - DNA, 40 bp, Artificial Sequence
  Description of Artificial Sequence Synthetic primer
ctaagaggcgcgccgaaacgatgtcctttgatagagcatc

SEQ ID NO: 54 - DNA, 40 bp, Artificial Sequence
  Description of Artificial Sequence Synthetic primer
ctaagaggcgcgccgaaacgatgaatgctgtaaggggctt

SEQ ID NO: 55 - DNA, 40 bp, Artificial Sequence
  Description of Artificial Sequence Synthetic primer
ctaagaggcgcgccgaaacgatggataatccgtttggaca

SEQ ID NO: 56 - DNA, 40 bp, Artificial Sequence
  Description of Artificial Sequence Synthetic primer
ctaagaggcgcgccgaaacgatgtctacaacaaaaccaat

SEQ ID NO: 57 - DNA, 40 bp, Artificial Sequence
  Description of Artificial Sequence Synthetic primer
ctaagaggcgcgccgaaacgatgtcggaaccaaacaccaa

SEQ ID NO: 58 - DNA, 40 bp, Artificial Sequence
  Description of Artificial Sequence Synthetic primer
ctaagaggcgcgccgaaacgatgtctgaagactgggactc

SEQ ID NO: 59 - DNA, 40 bp, Artificial Sequence
  Description of Artificial Sequence Synthetic primer
ctaagaggcgcgccgaaacgatggttagagaaacaaagtt

SEQ ID NO: 60 - DNA, 40 bp, Artificial Sequence
  Description of Artificial Sequence Synthetic primer
ctaagaggcgcgccgaaacgatggatctgttggacactta

SEQ ID NO: 61 - DNA, 40 bp, Artificial Sequence
  Description of Artificial Sequence Synthetic primer
ctaagaggcgcgccgaaacgatgctcaaaagattattgca

SEQ ID NO: 62 - DNA, 40 bp, Artificial Sequence
  Description of Artificial Sequence Synthetic primer
ctaagaggcgcgccgaaacgatgcccgtagattcttctca

SEQ ID NO: 63 - DNA, 40 bp, Artificial Sequence
  Description of Artificial Sequence Synthetic primer
ctaagaggcgcgccgaaacgatgttgcttcgtcgtcagtt

SEQ ID NO: 64 - DNA, 40 bp, Artificial Sequence
  Description of Artificial Sequence Synthetic primer
ctaagaggcgcgccgaaacgatggcatttactccaggtag

SEQ ID NO: 65 - DNA, 40 bp, Artificial Sequence
  Description of Artificial Sequence Synthetic primer
ctaagaggcgcgccgaaacgatgaatatatgtgatgttat

SEQ ID NO: 66 - DNA, 40 bp, Artificial Sequence
  Description of Artificial Sequence Synthetic primer
ctaagaggcgcgccgaaacgatgggtaaggaaaagttgca

SEQ ID NO: 67 - DNA, 40 bp, Artificial Sequence
  Description of Artificial Sequence Synthetic primer
ctaagaggcgcgccgaaacgatgtcagacacaagcaatga

SEQ ID NO: 68 - DNA, 40 bp, Artificial Sequence
  Description of Artificial Sequence Synthetic primer
ctaagaggcgcgccgaaacgatgatccgcttccttatcac

SEQ ID NO: 69 - DNA, 40 bp, Artificial Sequence
  Description of Artificial Sequence Synthetic primer
ctaagaggcgcgccgaaacgatgtttcaaccccaactacc

SEQ ID NO: 70 - DNA, 40 bp, Artificial Sequence
  Description of Artificial Sequence Synthetic primer
ctaagaggcgcgccgaaacgatgactgtcacccagttctt

SEQ ID NO: 71 - DNA, 40 bp, Artificial Sequence
  Description of Artificial Sequence Synthetic primer
ctaagaggcgcgccgaaacgatgaagatctctaccattgc

SEQ ID NO: 72 - DNA, 40 bp, Artificial Sequence
  Description of Artificial Sequence Synthetic primer
ctaagaggcgcgccgaaacgatggcgaagtttcattgcga

SEQ ID NO: 73 - DNA, 40 bp, Artificial Sequence
  Description of Artificial Sequence Synthetic primer
ctaagaggcgcgccgaaacgatgtttgccgaaaagaaaag

SEQ ID NO: 74 - DNA, 40 bp, Artificial Sequence
  Description of Artificial Sequence Synthetic primer
ctaagaggcgcgccgaaacgatgtctgatttatccacatc

SEQ ID NO: 75 - DNA, 40 bp, Artificial Sequence
  Description of Artificial Sequence Synthetic primer
ctaagaggcgcgccgaaacgatggtattggcagatcttgg

SEQ ID NO: 76 - DNA, 40 bp, Artificial Sequence
  Description of Artificial Sequence Synthetic primer
ctaagaggcgcgccgaaacgatgtcgtctgatatcctgga

SEQ ID NO: 77 - DNA, 40 bp, Artificial Sequence
  Description of Artificial Sequence Synthetic primer
ctaagaggcgcgccgaaacgatgtcgtcgtcatcttcccc

SEQ ID NO: 78 - DNA, 40 bp, Artificial Sequence
  Description of Artificial Sequence Synthetic primer
ctaagaggcgcgccgaaacgatgacagccttaaccgaaga

SEQ ID NO: 79 - DNA, 40 bp, Artificial Sequence
  Description of Artificial Sequence Synthetic primer
ctaagaggcgcgccgaaacgatgtctttcgaacagccaat

SEQ ID NO: 80 - DNA, 40 bp, Artificial Sequence
  Description of Artificial Sequence Synthetic primer
ctaagaggcgcgccgaaacgatgagtattcaattgcgaga

SEQ ID NO: 81 - DNA, 40 bp, Artificial Sequence
  Description of Artificial Sequence Synthetic primer
ctaagaggcgcgccgaaacgatggttcgaaaactgaagca

SEQ ID NO: 82 - DNA, 40 bp, Artificial Sequence
  Description of Artificial Sequence Synthetic primer
ctaagaggcgcgccgaaacgatgtctcaagcaccagcatc

SEQ ID NO: 83 - DNA, 40 bp, Artificial Sequence
  Description of Artificial Sequence Synthetic primer
ctaagaggcgcgccgaaacgatgggtacaaagactcagaa

SEQ ID NO: 84 - DNA, 40 bp, Artificial Sequence
  Description of Artificial Sequence Synthetic primer
ctaagaggcgcgccgaaacgatgttgtaccaatgtttaga

SEQ ID NO: 85 - DNA, 40 bp, Artificial Sequence
  Description of Artificial Sequence Synthetic primer
ctaagaggcgcgccgaaacgatgaagtacgttttatccga

SEQ ID NO: 86 - DNA, 40 bp, Artificial Sequence
  Description of Artificial Sequence Synthetic primer
ctaagaggcgcgccgaaacgatggcaaaatggtacctggt

SEQ ID NO: 87 - DNA, 40 bp, Artificial Sequence
  Description of Artificial Sequence Synthetic primer
ctaagaggcgcgccgaaacgatgtctgcaagtacttacag

SEQ ID NO: 88 - DNA, 40 bp, Artificial Sequence
  Description of Artificial Sequence Synthetic primer
ctaagaggcgcgccgaaacgatgagccaagatatgaacta

SEQ ID NO: 89 - DNA, 40 bp, Artificial Sequence
  Description of Artificial Sequence Synthetic primer
ctaagaggcgcgccgaaacgatgcctgaaagtcgaacaag

SEQ ID NO: 90 - DNA, 40 bp, Artificial Sequence
  Description of Artificial Sequence Synthetic primer
ctaagaggcgcgccgaaacgatggagtggaaactgaaacc

SEQ ID NO: 91 - DNA, 40 bp, Artificial Sequence
  Description of Artificial Sequence Synthetic primer
ctaagaggcgcgccgaaacgatgactactgctcccccaac

SEQ ID NO: 92 - DNA, 40 bp, Artificial Sequence
  Description of Artificial Sequence Synthetic primer
ctaagaggcgcgccgaaacgatgggaagaaaagctaagga

SEQ ID NO: 93 - DNA, 40 bp, Artificial Sequence
  Description of Artificial Sequence Synthetic primer
ctaagaggcgcgccgaaacgatggaaacgatattgccttt

SEQ ID NO: 94 - DNA, 40 bp, Artificial Sequence
  Description of Artificial Sequence Synthetic primer
ctaagaggcgcgccgaaacgatgtcattagccagaagtat

SEQ ID NO: 95 - DNA, 40 bp, Artificial Sequence
  Description of Artificial Sequence Synthetic primer
ctaagaggcgcgccgaaacgatggaaggaaacagaagaaa

SEQ ID NO: 96 - DNA, 40 bp, Artificial Sequence
  Description of Artificial Sequence Synthetic primer
ctaagaggcgcgccgaaacgatgagatgtaacccgttcga

SEQ ID NO: 97 - DNA, 40 bp, Artificial Sequence
  Description of Artificial Sequence Synthetic primer
ctaagaggcgcgccgaaacgatggctacactaaataaacc

SEQ ID NO: 98 - DNA, 40 bp, Artificial Sequence
  Description of Artificial Sequence Synthetic primer
ctaagaggcgcgccgaaacgatgagttctgaatttgacat

SEQ ID NO: 99 - DNA, 40 bp, Artificial Sequence
  Description of Artificial Sequence Synthetic primer
ctaagaggcgcgccgaaacgatgacagatcagcaagcatc

SEQ ID NO: 100 - DNA, 40 bp, Artificial Sequence
  Description of Artificial Sequence Synthetic primer
ctaagaggcgcgccgaaacgatggaggaagctaagttgaa

SEQ ID NO: 101 - DNA, 40 bp, Artificial Sequence
  Description of Artificial Sequence Synthetic primer
ctaagaggcgcgccgaaacgatgggtagaaggaaaataga

SEQ ID NO: 102 - DNA, 40 bp, Artificial Sequence
  Description of Artificial Sequence Synthetic primer
ctaagaggcgcgccgaaacgatgagtgcgaaaccaaccag

SEQ ID NO: 103 - DNA, 40 bp, Artificial Sequence
  Description of Artificial Sequence Synthetic primer
ctaagaggcgcgccgaaacgatgccattactagaggaaat

SEQ ID NO: 104 - DNA, 40 bp, Artificial Sequence
  Description of Artificial Sequence Synthetic primer
ctaagaggcgcgccgaaacgatgaccactgcctatcccaa

SEQ ID NO: 105 - DNA, 40 bp, Artificial Sequence
  Description of Artificial Sequence Synthetic primer
ctaagaggcgcgccgaaacgatgactattaaaggcggtgt

SEQ ID NO: 106 - DNA, 40 bp, Artificial Sequence
  Description of Artificial Sequence Synthetic primer
ctaagaggcgcgccgaaacgatgacagccactgcagtgaa

SEQ ID NO: 107 - DNA, 40 bp, Artificial Sequence
  Description of Artificial Sequence Synthetic primer
ctaagaggcgcgccgaaacgatgtctgctgctagtgaatc

SEQ ID NO: 108 - DNA, 40 bp, Artificial Sequence
  Description of Artificial Sequence Synthetic primer
ctaagaggcgcgccgaaacgatggacgctactgtgggtaa

SEQ ID NO: 109 - DNA, 40 bp, Artificial Sequence
  Description of Artificial Sequence Synthetic primer
ctaagaggcgcgccgaaacgatgggtaaatcaaacggcaa

SEQ ID NO: 110 - DNA, 40 bp, Artificial Sequence
  Description of Artificial Sequence Synthetic primer
ctaagaggcgcgccgaaacgatgtcagtggatgaaaatat

SEQ ID NO: 111 - DNA, 40 bp, Artificial Sequence
  Description of Artificial Sequence Synthetic primer
ctaagaggcgcgccgaaacgatgaagagtccaactacgga

SEQ ID NO: 112 - DNA, 40 bp, Artificial Sequence
  Description of Artificial Sequence Synthetic primer
ctaagaggcgcgccgaaacgatggcagaccaacggcttga

SEQ ID NO: 113 - DNA, 40 bp, Artificial Sequence
  Description of Artificial Sequence Synthetic primer
ctaagaggcgcgccgaaacgatggatgtggattttgacaa

SEQ ID NO: 114 - DNA, 40 bp, Artificial Sequence
  Description of Artificial Sequence Synthetic primer
ctaagaggcgcgccgaaacgatgagtaaagcggcctcaac

SEQ ID NO: 115 - DNA, 40 bp, Artificial Sequence
  Description of Artificial Sequence Synthetic primer
ctaagaggcgcgccgaaacgatgatgccggaggaacaagt

SEQ ID NO: 116 - DNA, 40 bp, Artificial Sequence
  Description of Artificial Sequence Synthetic primer
ctaagaggcgcgccgaaacgatggaatcgcccttgcaatc

SEQ ID NO: 117 - DNA, 40 bp, Artificial Sequence
  Description of Artificial Sequence Synthetic primer
ctaagaggcgcgccgaaacgatggattttttagcagatct

SEQ ID NO: 118 - DNA, 40 bp, Artificial Sequence
  Description of Artificial Sequence Synthetic primer
ctaagaggcgcgccgaaacgatggcgaaaagtcccacgac

SEQ ID NO: 119 - DNA, 40 bp, Artificial Sequence
  Description of Artificial Sequence Synthetic primer
ctaagaggcgcgccgaaacgatgaacaacgatattacgtt

SEQ ID NO: 120 - DNA, 40 bp, Artificial Sequence
  Description of Artificial Sequence Synthetic primer
ctaagaggcgcgccgaaacgatgggattggctgaaccaaa

SEQ ID NO: 121 - DNA, 40 bp, Artificial Sequence
  Description of Artificial Sequence Synthetic primer
ctaagaggcgcgccgaaacgatgtcagatctttggtaagt

SEQ ID NO: 122 - DNA, 40 bp, Artificial Sequence
  Description of Artificial Sequence Synthetic primer
ctaagaggcgcgccgaaacgatggatgtttatcatcaatt

SEQ ID NO: 123 - DNA, 40 bp, Artificial Sequence
  Description of Artificial Sequence Synthetic primer
ctaagaggcgcgccgaaacgatggtgcttataaacggcgt

SEQ ID NO: 124 - DNA, 40 bp, Artificial Sequence
  Description of Artificial Sequence Synthetic primer
ctaagaggcgcgccgaaacgatggcgctgaaacatcatcc

SEQ ID NO: 125 - DNA, 40 bp, Artificial Sequence
  Description of Artificial Sequence Synthetic primer
ctaagaggcgcgccgaaacgatgattggagttagatcatt

SEQ ID NO: 126 - DNA, 40 bp, Artificial Sequence
  Description of Artificial Sequence Synthetic primer
ctaagaggcgcgccgaaacgatgttctcaaaactttccca

SEQ ID NO: 127 - DNA, 40 bp, Artificial Sequence
  Description of Artificial Sequence Synthetic primer
ctaagaggcgcgccgaaacgatggccaagagacataaaaa

SEQ ID NO: 128 - DNA, 40 bp, Artificial Sequence
  Description of Artificial Sequence Synthetic primer
ctaagaggcgcgccgaaacgatgaaagggcatgagctgcc

SEQ ID NO: 129 - DNA, 40 bp, Artificial Sequence
  Description of Artificial Sequence Synthetic primer
ctaagaggcgcgccgaaacgatggattacgagctggcaac

SEQ ID NO: 130 - DNA, 40 bp, Artificial Sequence
  Description of Artificial Sequence Synthetic primer
ctaagaggcgcgccgaaacgatgtcctttgtacaagatta

SEQ ID NO: 131 - DNA, 40 bp, Artificial Sequence
  Description of Artificial Sequence Synthetic primer
ctaagaggcgcgccgaaacgatgtccaaagaggccatcag

SEQ ID NO: 132 - DNA, 40 bp, Artificial Sequence
  Description of Artificial Sequence Synthetic primer
ctaagaggcgcgccgaaacgatggcacaagaaagaacaga

SEQ ID NO: 133 - DNA, 40 bp, Artificial Sequence
  Description of Artificial Sequence Synthetic primer
ctaagaggcgcgccgaaacgatgatcaagctgctagagat

SEQ ID NO: 134 - DNA, 40 bp, Artificial Sequence
  Description of Artificial Sequence Synthetic primer
ctaagaggcgcgccgaaacgatgcctgacgaaatggatat

SEQ ID NO: 135 - DNA, 40 bp, Artificial Sequence
  Description of Artificial Sequence Synthetic primer
ctaagaggcgcgccgaaacgatgtcacaaggaacaattta

SEQ ID NO: 136 - DNA, 34 bp, Artificial Sequence
  Description of Artificial Sequence Synthetic primer
ttcagtcctgcaggctatgcacccagcttgtcat

SEQ ID NO: 137 - DNA, 34 bp, Artificial Sequence
  Description of Artificial Sequence Synthetic primer
ttcagtcctgcaggttaactgtcaaaatttattg

SEQ ID NO: 138 - DNA, 34 bp, Artificial Sequence
  Description of Artificial Sequence Synthetic primer
ttcagtcctgcaggttactcgttgagcagcgaat

SEQ ID NO: 139 - DNA, 34 bp, Artificial Sequence
  Description of Artificial Sequence Synthetic primer
ttcagtcctgcaggtcattttcttcctttctttt

SEQ ID NO: 140 - DNA, 34 bp, Artificial Sequence
  Description of Artificial Sequence Synthetic primer
ttcagtcctgcaggctagtctatatgctcactaa

SEQ ID NO: 141 - DNA, 34 bp, Artificial Sequence
  Description of Artificial Sequence Synthetic primer
ttcagtcctgcaggtcattccaacggttgtctcg

SEQ ID NO: 142 - DNA, 34 bp, Artificial Sequence
  Description of Artificial Sequence Synthetic primer
ttcagtcctgcaggtcattgaacttcactgtagc

SEQ ID NO: 143 - DNA, 34 bp, Artificial Sequence
  Description of Artificial Sequence Synthetic primer
ttcagtcctgcaggtcaaggagatacggtaggag

SEQ ID NO: 144 - DNA, 34 bp, Artificial Sequence
  Description of Artificial Sequence Synthetic primer
ttcagtcctgcaggttacagaatgttgcttattc

SEQ ID NO: 145 - DNA, 34 bp, Artificial Sequence
  Description of Artificial Sequence Synthetic primer
ttcagtcctgcaggctaaatgctgaaagaaggta

SEQ ID NO: 146 - DNA, 34 bp, Artificial Sequence
  Description of Artificial Sequence Synthetic primer
ttcagtcctgcaggctacaaactttgtgcattat

SEQ ID NO: 147 - DNA, 34 bp, Artificial Sequence
  Description of Artificial Sequence Synthetic primer
ttcagtcctgcaggttagctttcattgacccatt

SEQ ID NO: 148 - DNA, 34 bp, Artificial Sequence
  Description of Artificial Sequence Synthetic primer
ttcagtcctgcaggtcagttaagaaagattgatg

SEQ ID NO: 149 - DNA, 34 bp, Artificial Sequence
  Description of Artificial Sequence Synthetic primer
ttcagtcctgcaggctataatcctgaacggaaaa

SEQ ID NO: 150 - DNA, 34 bp, Artificial Sequence
  Description of Artificial Sequence Synthetic primer
ttcagtcctgcaggttaactggagtaatatatct

SEQ ID NO: 151 - DNA, 34 bp, Artificial Sequence
  Description of Artificial Sequence Synthetic primer
ttcagtcctgcaggttaatgcgtcgaatgtcctc

SEQ ID NO: 152 - DNA, 34 bp, Artificial Sequence
  Description of Artificial Sequence Synthetic primer
ttcagtcctgcaggtcactgcttacggtgagtac

SEQ ID NO: 153 - DNA, 34 bp, Artificial Sequence
  Description of Artificial Sequence Synthetic primer
ttcagtcctgcaggtcattctcccttgatggctt

SEQ ID NO: 154 - DNA, 34 bp, Artificial Sequence
  Description of Artificial Sequence Synthetic primer
ttcagtcctgcaggttagttcttctttggaaata

SEQ ID NO: 155 - DNA, 34 bp, Artificial Sequence
  Description of Artificial Sequence Synthetic primer
ttcagtcctgcaggttactgagaagcacattgga

SEQ ID NO: 156 - DNA, 34 bp, Artificial Sequence
  Description of Artificial Sequence Synthetic primer
ttcagtcctgcaggtcatgagtgaaaaatcttat

SEQ ID NO: 157 - DNA, 34 bp, Artificial Sequence
  Description of Artificial Sequence Synthetic primer
ttcagtcctgcaggctaattagtaggctttgctt

SEQ ID NO: 158 - DNA, 34 bp, Artificial Sequence
  Description of Artificial Sequence Synthetic primer
ttcagtcctgcaggtcacctgatcgctatgcatg

SEQ ID NO: 159 - DNA, 34 bp, Artificial Sequence
  Description of Artificial Sequence Synthetic primer
ttcagtcctgcaggttaggcgatgattctagtga

SEQ ID NO: 160 - DNA, 34 bp, Artificial Sequence
  Description of Artificial Sequence Synthetic primer
ttcagtcctgcaggttacaaaccgcttctcatat

SEQ ID NO: 161 - DNA, 34 bp, Artificial Sequence
  Description of Artificial Sequence Synthetic primer
ttcagtcctgcaggtcagtagtatggataatagt

SEQ ID NO: 162 - DNA, 34 bp, Artificial Sequence
  Description of Artificial Sequence Synthetic primer
ttcagtcctgcaggctatttcttagcggcctttt

SEQ ID NO: 163 - DNA, 34 bp, Artificial Sequence
  Description of Artificial Sequence Synthetic primer
ttcagtcctgcaggttaagtgtgatcatcgttca

SEQ ID NO: 164 - DNA, 34 bp, Artificial Sequence
  Description of Artificial Sequence Synthetic primer
ttcagtcctgcaggttactcggccgattgaacag

SEQ ID NO: 165 - DNA, 34 bp, Artificial Sequence
  Description of Artificial Sequence Synthetic primer
ttcagtcctgcaggttactgcgcttgttctgtgg

SEQ ID NO: 166 - DNA, 34 bp, Artificial Sequence
  Description of Artificial Sequence Synthetic primer
ttcagtcctgcaggctattccttccatcctagac

SEQ ID NO: 167 - DNA, 34 bp, Artificial Sequence
  Description of Artificial Sequence Synthetic primer
ttcagtcctgcaggctaggttctctttgtagctt

SEQ ID NO: 168 - DNA, 34 bp, Artificial Sequence
  Description of Artificial Sequence Synthetic primer
ttcagtcctgcaggttagcttgcactattgatgt

SEQ ID NO: 169 - DNA, 34 bp, Artificial Sequence
  Description of Artificial Sequence Synthetic primer
ttcagtcctgcaggctacagcaaaaaatgacccg

SEQ ID NO: 170 - DNA, 34 bp, Artificial Sequence
  Description of Artificial Sequence Synthetic primer
ttcagtcctgcaggtcaagctctggatggagatc

SEQ ID NO: 171 - DNA, 34 bp, Artificial Sequence
  Description of Artificial Sequence Synthetic primer
ttcagtcctgcaggttagcccataccaaactgtt

SEQ ID NO: 172 - DNA, 34 bp, Artificial Sequence
  Description of Artificial Sequence Synthetic primer
ttcagtcctgcaggctatgattctttatcttctt

SEQ ID NO: 173 - DNA, 34 bp, Artificial Sequence
  Description of Artificial Sequence Synthetic primer
ttcagtcctgcaggtcagactggtgaaggatttg

SEQ ID NO: 174 - DNA, 34 bp, Artificial Sequence
  Description of Artificial Sequence Synthetic primer
ttcagtcctgcaggtcagtttttagggatgttct

SEQ ID NO: 175 - DNA, 34 bp, Artificial Sequence
  Description of Artificial Sequence Synthetic primer
ttcagtcctgcaggctatagatgcgtcatggtaa

SEQ ID NO: 176 - DNA, 34 bp, Artificial Sequence
  Description of Artificial Sequence Synthetic primer
ttcagtcctgcaggctatatgtgggctacagagt

SEQ ID NO: 177 - DNA, 34 bp, Artificial Sequence
  Description of Artificial Sequence Synthetic primer
ttcagtcctgcaggttaactgaagtcaaaatcat

SEQ ID NO: 178 - DNA, 34 bp, Artificial Sequence
  Description of Artificial Sequence Synthetic primer
ttcagtcctgcaggttaatatctctttctgtaag

SEQ ID NO: 179 - DNA, 34 bp, Artificial Sequence
  Description of Artificial Sequence Synthetic primer
ttcagtcctgcaggtcaatgcttcttcttgttct

SEQ ID NO: 180 - DNA, 34 bp, Artificial Sequence
  Description of Artificial Sequence Synthetic primer
ttcagtcctgcaggtcaactattcgccgtatccg

SEQ ID NO: 181 - DNA, 34 bp, Artificial Sequence
  Description of Artificial Sequence Synthetic primer
ttcagtcctgcaggttattcttcttgaacgatag

SEQ ID NO: 182 - DNA, 34 bp, Artificial Sequence
  Description of Artificial Sequence Synthetic primer
ttcagtcctgcaggttaggttgaaggttgtttgt

SEQ ID NO: 183 - DNA, 34 bp, Artificial Sequence
  Description of Artificial Sequence Synthetic primer
ttcagtcctgcaggtcactgatgtttcactaaac

SEQ ID NO: 184 - DNA, 34 bp, Artificial Sequence
  Description of Artificial Sequence Synthetic primer
ttcagtcctgcaggttaggatatctgttgactaa

SEQ ID NO: 185 - DNA, 34 bp, Artificial Sequence
  Description of Artificial Sequence Synthetic primer
ttcagtcctgcaggctaggaatctagtttcatga

SEQ ID NO: 186 - DNA, 34 bp, Artificial Sequence
  Description of Artificial Sequence Synthetic primer
ttcagtcctgcaggtcaagaatcatatgtatatt

SEQ ID NO: 187 - DNA, 34 bp, Artificial Sequence
  Description of Artificial Sequence Synthetic primer
ttcagtcctgcaggtcaagagtccgagttcatga

SEQ ID NO: 188 - DNA, 34 bp, Artificial Sequence
  Description of Artificial Sequence Synthetic primer
ttcagtcctgcaggtcatagttgtatattctttg

SEQ ID NO: 189 - DNA, 34 bp, Artificial Sequence
  Description of Artificial Sequence Synthetic primer
ttcagtcctgcaggctaaatgagatcttcaacga

SEQ ID NO: 190 - DNA, 34 bp, Artificial Sequence
  Description of Artificial Sequence Synthetic primer
ttcagtcctgcaggctaaaagtcaattactattc

SEQ ID NO: 191 - DNA, 34 bp, Artificial Sequence
  Description of Artificial Sequence Synthetic primer
ttcagtcctgcaggttatataccaaacttaagcc

SEQ ID NO: 192 - DNA, 34 bp, Artificial Sequence
  Description of Artificial Sequence Synthetic primer
ttcagtcctgcaggtcatggagattcactctttt

SEQ ID NO: 193 - DNA, 34 bp, Artificial Sequence
  Description of Artificial Sequence Synthetic primer
ttcagtcctgcaggctagaacaagttctctactg

SEQ ID NO: 194 - DNA, 34 bp, Artificial Sequence
  Description of Artificial Sequence Synthetic primer
ttcagtcctgcaggttaatcataccacattttaa

SEQ ID NO: 195 - DNA, 34 bp, Artificial Sequence
  Description of Artificial Sequence Synthetic primer
ttcagtcctgcaggttaactattaaatttgagat

SEQ ID NO: 196 - DNA, 34 bp, Artificial Sequence
  Description of Artificial Sequence Synthetic primer
ttcagtcctgcaggttaggatggattgaaaggaa

SEQ ID NO: 197 - DNA, 34 bp, Artificial Sequence
  Description of Artificial Sequence Synthetic primer
ttcagtcctgcaggtcagctcttcttagtcacac

SEQ ID NO: 198 - DNA, 34 bp, Artificial Sequence
  Description of Artificial Sequence Synthetic primer
ttcagtcctgcaggctatttgcttataagatcag

SEQ ID NO: 199 - DNA, 34 bp, Artificial Sequence
  Description of Artificial Sequence Synthetic primer
ttcagtcctgcaggttatcttctcaccattattt

SEQ ID NO: 200 - DNA, 34 bp, Artificial Sequence
  Description of Artificial Sequence Synthetic primer
ttcagtcctgcaggtcatttcttttcttgatttt

SEQ ID NO: 201 - DNA, 34 bp, Artificial Sequence
  Description of Artificial Sequence Synthetic primer
ttcagtcctgcaggttaatttatcaggccgagag

SEQ ID NO: 202 - DNA, 34 bp, Artificial Sequence
  Description of Artificial Sequence Synthetic primer
ttcagtcctgcaggtcaccaaacaatacaataca

SEQ ID NO: 203 - DNA, 34 bp, Artificial Sequence
  Description of Artificial Sequence Synthetic primer
ttcagtcctgcaggctaaaactcctcatcggagg

SEQ ID NO: 204 - DNA, 34 bp, Artificial Sequence
  Description of Artificial Sequence Synthetic primer
ttcagtcctgcaggtcatttagaaaaatgggttt

SEQ ID NO: 205 - DNA, 34 bp, Artificial Sequence
  Description of Artificial Sequence Synthetic primer
ttcagtcctgcaggttaaatatcgtcaatatcaa

SEQ ID NO: 206 - DNA, 34 bp, Artificial Sequence
  Description of Artificial Sequence Synthetic primer
ttcagtcctgcaggttaagcaattcccttaagtg

SEQ ID NO: 207 - DNA, 34 bp, Artificial Sequence
  Description of Artificial Sequence Synthetic primer
ttcagtcctgcaggtcatgtgaatgccatctcat

SEQ ID NO: 208 - DNA, 34 bp, Artificial Sequence
  Description of Artificial Sequence Synthetic primer
ttcagtcctgcaggtcagtcatctatgttctgac

SEQ ID NO: 209 - DNA, 34 bp, Artificial Sequence
  Description of Artificial Sequence Synthetic primer
ttcagtcctgcaggtcagtactgtatatcgtaca

SEQ ID NO: 210 - DNA, 34 bp, Artificial Sequence
  Description of Artificial Sequence Synthetic primer
ttcagtcctgcaggctagaacactttggacaaaa

SEQ ID NO: 211 - DNA, 34 bp, Artificial Sequence
  Description of Artificial Sequence Synthetic primer
ttcagtcctgcaggctaaagtccgaataaactcc

SEQ ID NO: 212 - DNA, 34 bp, Artificial Sequence
  Description of Artificial Sequence Synthetic primer
ttcagtcctgcaggctagcggaacaatccaaaca

SEQ ID NO: 213 - DNA, 34 bp, Artificial Sequence
  Description of Artificial Sequence Synthetic primer
ttcagtcctgcaggctacgccttccgtatatgct

SEQ ID NO: 214 - DNA, 34 bp, Artificial Sequence
  Description of Artificial Sequence Synthetic primer
ttcagtcctgcaggttagtcttcaatagcactgg

SEQ ID NO: 215 - DNA, 34 bp, Artificial Sequence
  Description of Artificial Sequence Synthetic primer
ttcagtcctgcaggttacagaattttcttcgaag

SEQ ID NO: 216 - DNA, 34 bp, Artificial Sequence
  Description of Artificial Sequence Synthetic primer
ttcagtcctgcaggttactgagcgaccatgaata

SEQ ID NO: 217 - DNA, 34 bp, Artificial Sequence
  Description of Artificial Sequence Synthetic primer
ttcagtcctgcaggttaacaagtgacatcttggg

SEQ ID NO: 218 - DNA, 34 bp, Artificial Sequence
  Description of Artificial Sequence Synthetic primer
ttcagtcctgcaggctagagctcctgtagattca

SEQ ID NO: 219 - DNA, 34 bp, Artificial Sequence
  Description of Artificial Sequence Synthetic primer
ttcagtcctgcaggttaacggttcaatgggaccc

SEQ ID NO: 220 - DNA, 34 bp, Artificial Sequence
  Description of Artificial Sequence Synthetic primer
ttcagtcctgcaggctagcgtttctttttagatt

SEQ ID NO: 221 - DNA, 34 bp, Artificial Sequence
  Description of Artificial Sequence Synthetic primer
ttcagtcctgcaggctatttaatctttttgtcct

SEQ ID NO: 222 - DNA, 34 bp, Artificial Sequence
  Description of Artificial Sequence Synthetic primer
ttcagtcctgcaggttattttaatgagctggcta

SEQ ID NO: 223 - DNA, 34 bp, Artificial Sequence
  Description of Artificial Sequence Synthetic primer
ttcagtcctgcaggtcattcttttctcaatccca

SEQ ID NO: 224 - DNA, 34 bp, Artificial Sequence
  Description of Artificial Sequence Synthetic primer
ttcagtcctgcaggttaaagagccatagtcagcc

SEQ ID NO: 225 - DNA, 34 bp, Artificial Sequence
  Description of Artificial Sequence Synthetic primer
ttcagtcctgcaggttattgatcttgttcgtcgt

SEQ ID NO: 226 - DNA, 34 bp, Artificial Sequence
  Description of Artificial Sequence Synthetic primer
ttcagtcctgcaggctacccaaatacccttaata

SEQ ID NO: 227 - DNA, 34 bp, Artificial Sequence
  Description of Artificial Sequence Synthetic primer
ttcagtcctgcaggctaagtactggaaagccaat

SEQ ID NO: 228 - DNA, 34 bp, Artificial Sequence
  Description of Artificial Sequence Synthetic primer
ttcagtcctgcaggttatgactctactgaattgg

SEQ ID NO: 229 - DNA, 34 bp, Artificial Sequence
  Description of Artificial Sequence Synthetic primer
ttcagtcctgcaggttagaatattggcagaaacc

SEQ ID NO: 230 - DNA, 34 bp, Artificial Sequence
  Description of Artificial Sequence Synthetic primer
ttcagtcctgcaggttatccgtctgtttcaactt

SEQ ID NO: 231 - DNA, 34 bp, Artificial Sequence
  Description of Artificial Sequence Synthetic primer
ttcagtcctgcaggctaattgcccttaggaggag

SEQ ID NO: 232 - DNA, 9674 bp, Artificial Sequence
  Description of Artificial Sequence Synthetic polynucleotide
gcgatcgcggtctcactatccagaaaacggaacagattatggaattagtcaaaccaattgtcgacaatgttcgtcaaaatggtgacaaagcccttttagaactaactgccaagtttgatggagtcgctttgaagacacctgtgttagaagctcctttcccagaggaacttatgcaattgccagataacgttaagagagccattgatctctctatagataacgtcaggaaattccatgaagctcaactaacggagacgttgcaagttgagacttgccctggtgtagtctgctctcgttttgcaagacctattgagaaagttggcctctatattcctggtggaaccgcaattctgccttccacttccctgatgctgggtgttcctgccaaagttgctggttgcaaagaaattgtttttgcatctccacctaagaaggatggtacccttaccccagaagtcatctacgttgcccacaaggttggtgctaagtgtatcgtgctagcaggaggcgcccaggcagtagctgctatggcttacggaacagaaactgttcctaagtgtgacaaaatatttggtccaggaaaccagttcgttactgctgccaagatgatggttcaaaatgacacatcagccctgtgtagtattgacatgcctgctgggccttctgaagttctagttattgctgataaatacgctgatccagatttcgttgcctcagaccttctgtctcaagctgaacatggtattgattcccaggtgattctgttggctgtcgatatgacagacaaggagcttgccagaattgaagatgctgttcacaaccaagctgtgcagttgccaagggttgaaattgtacgcaagtgtattgcacactctacaaccctatcggttgcaacctacgagcaggctttggaaatgtccaatcagtacgctcctgaacacttgatcctgcaaatcgagaatgcttcttcttatgttgatcaagtacaacacgctggatctgtgtttgttggtgcctactctccagagagttgtggagattactcctccggtaccaaccacactttgccaacgtacggatatgcccgtcaatacagcggagttaacactgcaaccttccagaagttcatcacttcacaagacgtaactcctgagggactgaaacatattggccaagcagtgatggatctggctgctgttgaaggtctagatgctcaccgcaatgctgttaaggttcgtatggagaaactgggacttatttaattatttagagattttaacttacatttagattcgatagatctaaccggcatctaacatccagtgtttctaatctagtgaatcgagatcaagtctcactctgaacataataaataaggtctaaccccatggaattacatgtccccttgggttctgagattgtcttacccggtggggaaagagccatactcaagttcgtaggaaatgtggagtcaaagtctgggatttttgctggtgttgaactgattggtcaaatgagtggaaaaggaaaggcctcgatggcccagatctagggagggcatcattgaggtttccacaaaaggaagaaacatggatccagagacatcaacagagaggaaagcgggtagtgaagccgaagccacaacacagcccgatttggaagggagttcacaatcaaggtgagtccagccattttttttcttttttttttttttattcaggtgaacccacctaactatttttaactgggatccagtgagctcgctgggtgaaagccaaccatcttttgtttcggggaaccgtgctcgccccgtaaagttaatttttttttcccgcgcagctttaatctttcggcagagaaggcgttttcatcgtagcgtgggaacagaataatcagttcatgtgctatacaggcacatggcagcagtcactattttgctttttaaccttaaagtcgttcatcaatcattaactgaccaatcagattttttgcatttgccacttatctaaaaatacttttgtatctcgcagatacgttcagtggtttccaggacaacacccaaaaaaaggtatcaatgccactaggcagtcggttttatttttggtcacccacgcaaagaagcacccacctcttttaggttttaagttgtgggaacagtaacaccgcctagagcttcaggaaaaaccagtacctgtgaccgcaattcaccatgatgcagaatgttaatttaaacgagtgccaaatcaagatttcaacagacaaatcaatcgatccatagttacccattccagccttttcgtcgtcgagcctgcttcattcctgcctcaggtgcataactttgcatgaaaagtccagattagggcagattttgagtttaaaataggaaatataaacaaatataccgcgaaaaaggtttgtttatagcttttcgcctggtgccgtacggtataaatacatactctcctcccccccctggttctctttttcttttgttacttacattttaccgttccgtcactcgcttcactcaacaacaaaaggcgcgccgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagcaggtggttacggtccaggcgctggtcaacaaggtccaggaagtggtggtcaacaaggacctggcggtcaaggaccctacggtagtggccaacaaggtccaggtggagcaggacagcagggtccgggaggccaaggaccttacggaccaggtgctgctgctgccgccgctgccgctgccggaggttacggtccaggagccggacaacagggtccaggtggagctggacaacaaggtccaggatcacaaggtcctggtggacaaggtccatacggtcctggtgctggtcaacagggaccaggtagtcaaggacctggttcaggtggtcagcagggtccaggaggacagggtccttacggcccttctgccgctgcagcagcagccgctgccgcaggaggatacggacctggtgctggacaacgatctcaaggaccaggaggacaaggtccttatggacctggcgctggccaacaaggacctggttctcagggtccaggttcaggaggccaacaaggcccaggaggtcaaggaccatacggaccatccgctgcggcagctgcagctgctgcaggtggatatggcccaggagccggacaacagggtcctggttcacaaggtccaggatctggtggtcaacagggaccaggcggccagggaccttatggtccaggagccgctgcagcagcagcagctgttggaggttacggccctggtgccggtcaacaaggcccaggatctcagggtcctggatctggaggacaacaaggtcctggaggtcagggtccatacggaccttcagcagcagctgctgctgcagccgctggtggttatggacctggtgctggtcaacaaggaccgggttctcagggtccgggttcaggaggtcagcagggccctggtggacaaggaccttatggacctagtgcggctgcagcagctgccgccgcaggtggttacggtccaggcgctggtcaacaaggtccaggaagtggtggtcaacaaggacctggcggtcaaggaccctacggtagtggccaacaaggtccaggtggagcaggacagcagggtccgggaggccaaggaccttacggaccaggtgctgctgctgccgccgctgccgctgccggaggttacggtccaggagccggacaacagggtccaggtggagctggacaacaaggtccaggatcacaaggtcctggtggacaaggtccatacggtcctggtgctggtcaacagggaccaggtagtcaaggacctggttcaggtggtcagcagggtccaggaggacagggtccttacggcccttctgccgctgcagcagcagccgctgccgcaggaggatacggacctggtgctggacaacgatctcaaggaccaggaggacaaggtccttatggacctggcgctggccaacaaggacctggttctcagggtccaggttcaggaggccaacaaggcccaggaggtcaaggaccatacggaccatccgctgcggcagctgcagctgctgcaggtggatatggcccaggagccggacaacagggtcctggttcacaaggtccaggatctggtggtcaacagggaccaggcggccagggaccttatggtccaggagccgctgcagcagcagcagctgttggaggttacggccctggtgccggtcaacaaggcccaggatctcagggtcctggatctggaggacaacaaggtcctggaggtcagggtccatacggaccttcagcagcagctgctgctgcagccgctggtggttatggacctggtgctggtcaacaaggaccgggttctcagggtccgggttcaggaggtcagcagggccctggtggacaaggaccttatggacctagtgcggctgcagcagctgccgccgcaggtggttacggtccaggcgctggtcaacaaggtccaggaagtggtggtcaacaaggacctggcggtcaaggaccctacggtagtggccaacaaggtccaggtggagcaggacagcagggtccgggaggccaaggaccttacggaccaggtgctgctgctgccgccgctgccgctgccggaggttacggtccaggagccggacaacagggtccaggtggagctggacaacaaggtccaggatcacaaggtcctggtggacaaggtccatacggtcctggtgctggtcaacagggaccaggtagtcaaggacctggttcaggtggtcagcagggtccaggaggacagggtccttacggcccttctgccgctgcagcagcagccgctgccgcaggaggatacggacctggtgctggacaacgatctcaaggaccaggaggacaaggtccttatggacctggcgctggccaacaaggacctggttctcagggtccaggttcaggaggccaacaaggcccaggaggtcaaggaccatacggaccatccgctgcggcagctgcagctgctgcaggtggatatggcccaggagccggacaacagggtcctggttcacaaggtccaggatctggtggtcaacagggaccaggcggccagggaccttatggtccaggagccgctgcagcagcagcagctgttggaggttacggccctggtgccggtcaacaaggcccaggatctcagggtcctggatctggaggacaacaaggtcctggaggtcagggtccatacggaccttcagcagcagctgctgctgcagccgctggtggttatggacctggtgctggtcaacaaggaccgggttctcagggtccgggttcaggaggtcagcagggccctggtggacaaggaccttatggacctagtgcggctgcagcagctgccgccgcaggtaccgcactaacagaaggagctaaactattcgaaaaggagattccttacattacagaattagagggtgatgtcgaaggaatgaaattcattatcaagggcgagggtactggtgacgctactaccggtacgattaaagcaaagtacatctgtacaacaggtgaccttcctgttccgtgggctactctggtgagcactttgtcttatggagttcaatgttttgctaaatacccttcgcacattaaagactttttcaaaagtgcaatgcctgagggctatactcaggagagaacaatatctttcgaaggagatggtgtgtataagactagggctatggtcacgtatgaaagaggatccatctacaatagagtaactttaactggtgaaaacttcaaaaaggacggtcacatccttagaaagaatgttgcctttcaatgcccaccatccatcttgtacattttgccagacacagttaacaatggtatcagagttgagtttaaccaagcttatgacatagagggtgtcaccgaaaagttggttacaaaatgttcacagatgaatcgtcccctggcaggatcagctgccgtccatatcccacgttaccatcatatcacttatcataccaagctgtccaaagatcgtgatgagagaagggatcacatgtgtttggttgaagtggtaaaggccgtggatttggatacttaccaataaggtacgtcttcatcgctatcctgcaggagacatgactgttcctcagttcaagttgggcacttacgagaagaccggtcttgctagattctaatcaagaggatgtcagaatgccatttgcctgagagatgcaggcttcatttttgattacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaggatccctggaccacaggtatctgatgcggccgcgcaaagttggtagatgtgacttccactgttgcttctggtataatccccattattgatgctcggcaattgactactgaatacgaactttctgaagatgtcaaaaagttccctgtcagtgaaattttgttggcgtctttgactactgaccgccccgatggtctattcactactttggtggctgactcttctaattactcgttgggcctggtgtactcgtccaaaaagtctattccggaggctataaggacacaaactggagtctaccaatctcgtcgtcacggtttgtggtataaaggtgctacatctggagcaactcaaaagttgctgggtatcgaattggattgtgatggagactgcttgaaatttgtggttgaacaaacaggtgttggtttctgtcacttggaacgcacttcctgttttggccaatcaaagggtcttagagccatggaagccaccttgtgggatcgtaagagcaatgctccagaaggttcttataccaaacggttatttgacgacgaagttttgttgaacgctaaaattagggaggaagctgatgaacttgcagaagctaaatccaaggaagatatagcctgggaatgtgctgacttattttattttgcattagttagatgtgccaagtacggtgtgacgttggacgaggtggagagaaacctggatatgaagtccctaaaggtcactagaaggaaaggagatgccaagccaggatacaccaaggaacaacctaaagaagaatccaaacctaaagaagtcccttctgaaggtcgtattgaattgtgcaaaattgacgtttctaaggcctcctcacaagaaattgaagatgcccttcgtcgtcctatagagaccttaattaagcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaagacgtcccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggtgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataactgtcagaccaagtttactcatatatactttagattgatttaaaacttcatttttaatttaaaaggatctaggtgaagatcctt

SEQ ID NO: 233 - DNA, 10198 bp, Artificial Sequence
  Description of Artificial Sequence Synthetic polynucleotide
gcgatcgcggtctcacagatgacagagttgtcaagaacttgaccactttgttgttcgacacagctttgttgacttccggtttcactttggatgagccaacttctttcgctgccagaatcaacggtttgatctccattggtttgaacatcgatgaggaggaagagaaagagccagaacaggctactgaagctccaagtgaagaagctgttgctgagtctgccatggaggaggttgactagttgaatttaggtatatatagtgactgtgatatttagctaatgaaatctaattggatatttagaatgcctcatctcgtagcctatcaattactattaggccatctcttatgggcccttctttgaaattgcattcaaggggggatgggactattttgaatttgaagtttggactctgtgagctgtttggccaattgaagtcatccacttgtacacagggattcaccagtagtttagaacaattctctatcgttattctcttgtcgtctttggcaatacaagcgtcgatgactgagttggtgactttatgaagtctaagttgatatgagtttgaaattatgaaacagttttttacactggacatgtagatagggcccttgatgtttaggaagaggatacagtttgagatgttggagatgtgtgtggagggagcgaccacttttaaaaccacatgatccagacgttgctcagttatcgaagtttcggaaacaaggcctcgatggcccagatctagggagggcatcattgaggtttccacaaaaggaagaaacatggatccagagacatcaacagagaggaaagcgggtagtgaagccgaagccacaacacagcccgatttggaagggagttcacaatcaaggtgagtccagccattttttttcttttttttttttttattcaggtgaacccacctaactatttttaactgggatccagtgagctcgctgggtgaaagccaaccatcttttgtttcggggaaccgtgctcgccccgtaaagttaatttttttttcccgcgcagctttaatctttcggcagagaaggcgttttcatcgtagcgtgggaacagaataatcagttcatgtgctatacaggcacatggcagcagtcactattttgctttttaaccttaaagtcgttcatcaatcattaactgaccaatcagattttttgcatttgccacttatctaaaaatacttttgtatctcgcagatacgttcagtggtttccaggacaacacccaaaaaaaggtatcaatgccactaggcagtcggttttatttttggtcacccacgcaaagaagcacccacctcttttaggttttaagttgtgggaacagtaacaccgcctagagcttcaggaaaaaccagtacctgtgaccgcaattcaccatgatgcagaatgttaatttaaacgagtgccaaatcaagatttcaacagacaaatcaatcgatccatagttacccattccagccttttcgtcgtcgagcctgcttcattcctgcctcaggtgcataactttgcatgaaaagtccagattagggcagattttgagtttaaaataggaaatataaacaaatataccgcgaaaaaggtttgtttatagcttttcgcctggtgccgtacggtataaatacatactctcctcccccccctggttctctttttcttttgttacttacattttaccgttccgtcactcgcttcactcaacaacaaaaggcgcgccgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagcaggtggttacggtccaggcgctggtcaacaaggtccaggaagtggtggtcaacaaggacctggcggtcaaggaccctacggtagtggccaacaaggtccaggtggagcaggacagcagggtccgggaggccaaggaccttacggaccaggtgctgctgctgccgccgctgccgctgccggaggttacggtccaggagccggacaacagggtccaggtggagctggacaacaaggtccaggatcacaaggtcctggtggacaaggtccatacggtcctggtgctggtcaacagggaccaggtagtcaaggacctggttcaggtggtcagcagggtccaggaggacagggtccttacggcccttctgccgctgcagcagcagccgctgccgcaggaggatacggacctggtgctggacaacgatctcaaggaccaggaggacaaggtccttatggacctggcgctggccaacaaggacctggttctcagggtccaggttcaggaggccaacaaggcccaggaggtcaaggaccatacggaccatccgctgcggcagctgcagctgctgcaggtggatatggcccaggagccggacaacagggtcctggttcacaaggtccaggatctggtggtcaacagggaccaggcggccagggaccttatggtccaggagccgctgcagcagcagcagctgttggaggttacggccctggtgccggtcaacaaggcccaggatctcagggtcctggatctggaggacaacaaggtcctggaggtcagggtccatacggaccttcagcagcagctgctgctgcagccgctggtggttatggacctggtgctggtcaacaaggaccgggttctcagggtccgggttcaggaggtcagcagggccctggtggacaaggaccttatggacctagtgcggctgcagcagctgccgccgcaggtggttacggtccaggcgctggtcaacaaggtccaggaagtggtggtcaacaaggacctggcggtcaaggaccctacggtagtggccaacaaggtccaggtggagcaggacagcagggtccgggaggccaaggaccttacggaccaggtgctgctgctgccgccgctgccgctgccggaggttacggtccaggagccggacaacagggtccaggtggagctggacaacaaggtccaggatcacaaggtcctggtggacaaggtccatacggtcctggtgctggtcaacagggaccaggtagtcaaggacctggttcaggtggtcagcagggtccaggaggacagggtccttacggcccttctgccgctgcagcagcagccgctgccgcaggaggatacggacctggtgctggacaacgatctcaaggaccaggaggacaaggtccttatggacctggcgctggccaacaaggacctggttctcagggtccaggttcaggaggccaacaaggcccaggaggtcaaggaccatacggaccatccgctgcggcagctgcagctgctgcaggtggatatggcccaggagccggacaacagggtcctggttcacaaggtccaggatctggtggtcaacagggaccaggcggccagggaccttatggtccaggagccgctgcagcagcagcagctgttggaggttacggccctggtgccggtcaacaaggcccaggatctcagggtcctggatctggaggacaacaaggtcctggaggtcagggtccatacggaccttcagcagcagctgctgctgcagccgctggtggttatggacctggtgctggtcaacaaggaccgggttctcagggtccgggttcaggaggtcagcagggccctggtggacaaggaccttatggacctagtgcggctgcagcagctgccgccgcaggtggttacggtccaggcgctggtcaacaaggtccaggaagtggtggtcaacaaggacctggcggtcaaggaccctacggtagtggccaacaaggtccaggtggagcaggacagcagggtccgggaggccaaggaccttacggaccaggtgctgctgctgccgccgctgccgctgccggaggttacggtccaggagccggacaacagggtccaggtggagctggacaacaaggtccaggatcacaaggtcctggtggacaaggtccatacggtcctggtgctggtcaacagggaccaggtagtcaaggacctggttcaggtggtcagcagggtccaggaggacagggtccttacggcccttctgccgctgcagcagcagccgctgccgcaggaggatacggacctggtgctggacaacgatctcaaggaccaggaggacaaggtccttatggacctggcgctggccaacaaggacctggttctcagggtccaggttcaggaggccaacaaggcccaggaggtcaaggaccatacggaccatccgctgcggcagctgcagctgctgcaggtggatatggcccaggagccggacaacagggtcctggttcacaaggtccaggatctggtggtcaacagggaccaggcggccagggaccttatggtccaggagccgctgcagcagcagcagctgttggaggttacggccctggtgccggtcaacaaggcccaggatctcagggtcctggatctggaggacaacaaggtcctggaggtcagggtccatacggaccttcagcagcagctgctgctgcagccgctggtggttatggacctggtgctggtcaacaaggaccgggttctcagggtccgggttcaggaggtcagcagggccctggtggacaaggaccttatggacctagtgcggctgcagcagctgccgccgcaggtaccgcactaacagaaggagctaaactattcgaaaaggagattccttacattacagaattagagggtgatgtcgaaggaatgaaattcattatcaagggcgagggtactggtgacgctactaccggtacgattaaagcaaagtacatctgtacaacaggtgaccttcctgttccgtgggctactctggtgagcactttgtcttatggagttcaatgttttgctaaatacccttcgcacattaaagactttttcaaaagtgcaatgcctgagggctatactcaggagagaacaatatctttcgaaggagatggtgtgtataagactagggctatggtcacgtatgaaagaggatccatctacaatagagtaactttaactggtgaaaacttcaaaaaggacggtcacatccttagaaagaatgttgcctttcaatgcccaccatccatcttgtacattttgccagacacagttaacaatggtatcagagttgagtttaaccaagcttatgacatagagggtgtcaccgaaaagttggttacaaaatgttcacagatgaatcgtcccctggcaggatcagctgccgtccatatcccacgttaccatcatatcacttatcataccaagctgtccaaagatcgtgatgagagaagggatcacatgtgtttggttgaagtggtaaaggccgtggatttggatacttaccaataaggtacgtcttcatcgctatcctgcaggagacatgactgttcctcagttcaagttgggcacttacgagaagaccggtcttgctagattctaatcaagaggatgtcagaatgccatttgcctgagagatgcaggcttcatttttgattacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaggatccttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaatttttgacggctagctcagtcctaggtacgctagcattaaagaggagaaaatgactactcttgatgacacagcctacagatataggacatcagttccgggtgacgcagaggctatcgaagccttggacggttcattcactactgatacggtgtttagagtcaccgctacaggtgatggcttcaccttgagagaggttcctgtagacccacccttaacgaaagttttccctgatgacgaatcggatgacgagtctgatgctggtgaggacggtgaccctgattccagaacatttgtcgcatacggagatgatggtgacctggctggctttgttgtggtgtcctacagcggatggaatcgtagactcacagttgaggacatcgaagttgcacctgaacatcgtggtcacggtgttggtcgtgcactgatgggactggcaacagagtttgctagagaaagaggagccggacatttgtggttagaagtgaccaatgtcaacgctcctgctattcacgcatataggcgaatgggtttcactttgtgcggtcttgatactgctttgtatgacggaactgcttctgatggtgaacaagctctttacatgagtatgccatgtccatagcacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctgcggccgcaagaagttgattgagactttcaacgagattgctgaagacaaggaacaattcgagaagttttacagtgctttctccaagaacttgaagttgggtgtccatgaagacagccaaaacagatccgcattggccaagttgctgagatttaactccaccaagtctactgaggagctaacctcattctctgactacgtcaccagaatgccagagcaccagaagaacatctacttcattaccggtgagtctgtcaaggctcttgagaaatctccattcttggatgctttgaaggagaagaactttgaggtcctattgctgaccgatcctattgatgagtacgctatgactcaattgaaagagattgaggacaagaaattggttgacatcactaaagactttgagctggaagagtctgaggaggagaagaaggctagagaggaagaggttaaagatttcgagcctttgactaaagccctgaaagagattttgggtgacaaggttgagaaggttgtagtttcctacaagctggttgactctcctgctgctattagaacttcccaattcggctggtctgctaacatggaaagaattatgaaggctcaagctctgagagacaccaacaccatgtcctcgtacatggcttcaaagaagatcttcgagatctctccaaagtcgccaatcattaaggctttgagaaagaaggttgaggctaccggtacagaagagaccttaattaagcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaagacgtcccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggtgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataactgtcagaccaagtttactcatatatactttagattgatttaaaacttcatttttaatttaaaaggatctaggtgaagatcctt

SEQ ID NO: 234 - DNA, 10482 bp, Artificial Sequence
  Description of Artificial Sequence Synthetic polynucleotide
gcgatcgcggtctcatgatatatttgtattttgttcgttaacattgatgttttcttcatttactgttattgtttgtaactttgatcgatttatcttttctactttactgtaatatggctggcgggtgagccttgaactccctgtattactttaccttgctattacttaatctattgactagcagcgacctcttcaaccgaagggcaagtacacagcaagttcatgtctccgtaagtgtcatcaaccctggaaacagtgggccatgtcttttgctccttcaaaaatggcaatgggtaggctgcctcctctcttgtgtatcctctctgggaccactcagcgtcacttgtgctaataatatcttttaggttgtgtggggagttgtgcaagattgcaccatctgtttctccgttttctactttacggatttcttctctaatagagatcatagagtcaatgaatctgtctaattcttctttgtattcggattcagttggttctaccatcaatgtgcccggaatagggaacgacatggtaggagcatggaatccataatcttgcagacgcttggccacatcaatggcctcaattccgaatttcttgaatggtctaagatcaataatgaactcatggccacagtacttgtggcctcgatggcccagatctagggagggcatcattgaggtttccacaaaaggaagaaacatggatccagagacatcaacagagaggaaagcgggtagtgaagccgaagccacaacacagcccgatttggaagggagttcacaatcaaggtgagtccagccattttttttcttttttttttttttattcaggtgaacccacctaactatttttaactgggatccagtgagctcgctgggtgaaagccaaccatcttttgtttcggggaaccgtgctcgccccgtaaagttaatttttttttcccgcgcagctttaatctttcggcagagaaggcgttttcatcgtagcgtgggaacagaataatcagttcatgtgctatacaggcacatggcagcagtcactattttgctttttaaccttaaagtcgttcatcaatcattaactgaccaatcagattttttgcatttgccacttatctaaaaatacttttgtatctcgcagatacgttcagtggtttccaggacaacacccaaaaaaaggtatcaatgccactaggcagtcggttttatttttggtcacccacgcaaagaagcacccacctcttttaggttttaagttgtgggaacagtaacaccgcctagagcttcaggaaaaaccagtacctgtgaccgcaattcaccatgatgcagaatgttaatttaaacgagtgccaaatcaagatttcaacagacaaatcaatcgatccatagttacccattccagccttttcgtcgtcgagcctgcttcattcctgcctcaggtgcataactttgcatgaaaagtccagattagggcagattttgagtttaaaataggaaatataaacaaatataccgcgaaaaaggtttgtttatagcttttcgcctggtgccgtacggtataaatacatactctcctcccccccctggttctctttttcttttgttacttacattttaccgttccgtcactcgcttcactcaacaacaaaaggcgcgccgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagcaggtggttacggtccaggcgctggtcaacaaggtccaggaagtggtggtcaacaaggacctggcggtcaaggaccctacggtagtggccaacaaggtccaggtggagcaggacagcagggtccgggaggccaaggaccttacggaccaggtgctgctgctgccgccgctgccgctgccggaggttacggtccaggagccggacaacagggtccaggtggagctggacaacaaggtccaggatcacaaggtcctggtggacaaggtccatacggtcctggtgctggtcaacagggaccaggtagtcaaggacctggttcaggtggtcagcagggtccaggaggacagggtccttacggcccttctgccgctgcagcagcagccgctgccgcaggaggatacggacctggtgctggacaacgatctcaaggaccaggaggacaaggtccttatggacctggcgctggccaacaaggacctggttctcagggtccaggttcaggaggccaacaaggcccaggaggtcaaggaccatacggaccatccgctgcggcagctgcagctgctgcaggtggatatggcccaggagccggacaacagggtcctggttcacaaggtccaggatctggtggtcaacagggaccaggcggccagggaccttatggtccaggagccgctgcagcagcagcagctgttggaggttacggccctggtgccggtcaacaaggcccaggatctcagggtcctggatctggaggacaacaaggtcctggaggtcagggtccatacggaccttcagcagcagctgctgctgcagccgctggtggttatggacctggtgctggtcaacaaggaccgggttctcagggtccgggttcaggaggtcagcagggccctggtggacaaggaccttatggacctagtgcggctgcagcagctgccgccgcaggtggttacggtccaggcgctggtcaacaaggtccaggaagtggtggtcaacaaggacctggcggtcaaggaccctacggtagtggccaacaaggtccaggtggagcaggacagcagggtccgggaggccaaggaccttacggaccaggtgctgctgctgccgccgctgccgctgccggaggttacggtccaggagccggacaacagggtccaggtggagctggacaacaaggtccaggatcacaaggtcctggtggacaaggtccatacggtcctggtgctggtcaacagggaccaggtagtcaaggacctggttcaggtggtcagcagggtccaggaggacagggtccttacggcccttctgccgctgcagcagcagccgctgccgcaggaggatacggacctggtgctggacaacgatctcaaggaccaggaggacaaggtccttatggacctggcgctggccaacaaggacctggttctcagggtccaggttcaggaggccaacaaggcccaggaggtcaaggaccatacggaccatccgctgcggcagctgcagctgctgcaggtggatatggcccaggagccggacaacagggtcctggttcacaaggtccaggatctggtggtcaacagggaccaggcggccagggaccttatggtccaggagccgctgcagcagcagcagctgttggaggttacggccctggtgccggtcaacaaggcccaggatctcagggtcctggatctggaggacaacaaggtcctggaggtcagggtccatacggaccttcagcagcagctgctgctgcagccgctggtggttatggacctggtgctggtcaacaaggaccgggttctcagggtccgggttcaggaggtcagcagggccctggtggacaaggaccttatggacctagtgcggctgcagcagctgccgccgcaggtggttacggtccaggcgctggtcaacaaggtccaggaagtggtggtcaacaaggacctggcggtcaaggaccctacggtagtggccaacaaggtccaggtggagcaggacagcagggtccgggaggccaaggaccttacggaccaggtgctgctgctgccgccgctgccgctgccggaggttacggtccaggagccggacaacagggtccaggtggagctggacaacaaggtccaggatcacaaggtcctggtggacaaggtccatacggtcctggtgctggtcaacagggaccaggtagtcaaggacctggttcaggtggtcagcagggtccaggaggacagggtccttacggcccttctgccgctgcagcagcagccgctgccgcaggaggatacggacctggtgctggacaacgatctcaaggaccaggaggacaaggtccttatggacctggcgctggccaacaaggacctggttctcagggtccaggttcaggaggccaacaaggcccaggaggtcaaggaccatacggaccatccgctgcggcagctgcagctgctgcaggtggatatggcccaggagccggacaacagggtcctggttcacaaggtccaggatctggtggtcaacagggaccaggcggccagggaccttatggtccaggagccgctgcagcagcagcagctgttggaggttacggccctggtgccggtcaacaaggcccaggatctcagggtcctggatctggaggacaacaaggtcctggaggtcagggtccatacggaccttcagcagcagctgctgctgcagccgctggtggttatggacctggtgctggtcaacaaggaccgggttctcagggtccgggttcaggaggtcagcagggccctggtggacaaggaccttatggacctagtgcggctgcagcagctgccgccgcaggtaccgcactaacagaaggagctaaactattcgaaaaggagattccttacattacagaattagagggtgatgtcgaaggaatgaaattcattatcaagggcgagggtactggtgacgctactaccggtacgattaaagcaaagtacatctgtacaacaggtgaccttcctgttccgtgggctactctggtgagcactttgtcttatggagttcaatgttttgctaaatacccttcgcacattaaagactttttcaaaagtgcaatgcctgagggctatactcaggagagaacaatatctttcgaaggagatggtgtgtataagactagggctatggtcacgtatgaaagaggatccatctacaatagagtaactttaactggtgaaaacttcaaaaaggacggtcacatccttagaaagaatgttgcctttcaatgcccaccatccatcttgtacattttgccagacacagttaacaatggtatcagagttgagtttaaccaagcttatgacatagagggtgtcaccgaaaagttggttacaaaatgttcacagatgaatcgtcccctggcaggatcagctgccgtccatatcccacgttaccatcatatcacttatcataccaagctgtccaaagatcgtgatgagagaagggatcacatgtgtttggttgaagtggtaaaggccgtggatttggatacttaccaataaggtacgtcttcatcgctatcctgcaggagacatgactgttcctcagttcaagttgggcacttacgagaagaccggtcttgctagattctaatcaagaggatgtcagaatgccatttgcctgagagatgcaggcttcatttttgattacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaggatccttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaatttttgacggctagctcagtcctaggtacgctagcattaaagaggagaaaatgaaaaagccagagctgacagccacctctgtagaaaagtttttgattgagaaattcgactcagttagcgatctgatgcagttgtcggaaggtgaggaatcccgtgcattttccttcgatgttggtggacgtggttatgtccttcgtgttaattcctgcgccgacggtttctacaaagacagatacgtgtacaggcacttcgcttccgctgctttgccaattcctgaggtccttgatattggagaattttccgagtctttgacatattgtatttctaggcgagcacaaggtgttactctacaagatttgcctgaaacagaattgcccgcagtactacagccagtggccgaagctatggacgcaatagcagccgctgacttgagtcagacgagtggttttggtccatttggaccccaaggtatcggtcaatacactacttggagagacttcatctgtgcaattgctgatccgcatgtgtatcattggcaaacggttatggatgacactgtatctgcatccgttgctcaggctttggatgagctgatgctttgggccgaagattgtcctgaagtcagacacctggtacacgctgatttcggctcaaataatgtgttgaccgacaacggtaggatcacagcagtgatcgactggtcagaggcaatgtttggagattcacaatacgaggtggctaacatctttttctggcgtccttggctcgcatgcatggaacaacaaactcgttatttcgagagaaggcatccagagttagctggttctcctagacttagagcctacatgctgagaattggattagatcagttgtatcaaagcttagttgatggcaattttgatgacgctgcttgggctcaaggaagatgtgacgctatcgtcagaagtggtgcaggcactgtcggtagaacacaaatagcaagacgtagcgctgctgtttggactgacggatgtgttgaggttttagccgacagtggtaacaggcgtccatccacaagaccaagagctaaggaataacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctgcggccgcggtgtcatcaaggctggtatggtcgtcactttcgccccagctggtgtcactaccgaagtcaagtcggtcgagatgcaccacgagcaattggagcaaggtgtcccaggtgacaacgttggattcaacgtcaagaacgtttccgtcaaggaaatcagaagaggtaacgtctgtggtgactccaagaacgacccaccaaaggccgctgaatctttcaacgcccaggtcattatcttgaaccacccaggtcaaatctctgctggttacgctccagttttggactgtcacaccgctcacattgcttgtaagttcgacgagttgattgagaagattgacagaagaaccggtaagaagactgaggagaaccctaagttcatcaagtccggtgacgccgctatcgtcaagttggtcccatctaagccaatgtgtgttgaggccttcactgactacccacctttaggaagattcgctgtcagagacatgagacaaactgttgctgtcggtgttatcaagtccgttgtcaagactgacaaggctggtaaggtcaccaaggctgctcaaaaggccgctaagaaatagattgcttgaagctttaatttattttattaacataataataatacaagcatgatagagaccttaattaagcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaagacgtcccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggtgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataactgtcagaccaagtttactcatatatactttagattgatttaaaacttcatttttaatttaaaaggatctaggtgaagatcctt

SEQ ID NO: 235 - DNA, 931 bp, Artificial Sequence
  Description of Artificial Sequence Synthetic polynucleotide
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattgaaa

SEQ ID NO: 236 - DNA, 67 bp, Artificial Sequence
  Description of Artificial Sequence Synthetic oligonucleotide
caagggaatggtttgccaccgataacgggtcgatggtttactcgttaacagtatgtccctcttaatt

SEQ ID NO: 237 - DNA, 63 bp, Artificial Sequence
  Description of Artificial Sequence Synthetic oligonucleotide
ggttcagttaacatttgatcgatgttcttagaattggcagagagcttctgccctctagataga

SEQ ID NO: 238 - DNA, 56 bp, Artificial Sequence
  Description of Artificial Sequence Synthetic oligonucleotide
aaaatgatgaattttaccaagaacagttggtgacagattgtaaacgcctgttacat

SEQ ID NO: 239 - DNA, 63 bp, Artificial Sequence
  Description of Artificial Sequence Synthetic oligonucleotide
gggtcgatggtttactcgttaacagtcttagaattggcagagagcttctgccctctagataga

SEQ ID NO: 240 - DNA, 30 bp, Artificial Sequence
  Description of Artificial Sequence Synthetic oligonucleotide
acatcaaatggtttgccaccgataacaggg

SEQ ID NO: 241 - DNA, 87 bp, Artificial Sequence
  Description of Artificial Sequence Synthetic oligonucleotide
aaaatgatgaattttaccaagaacagttggtgacagattgtaaacgcctgttggttcagttaacatttgatcgatgttatgtccctt

SEQ ID NO: 242 - RNA, 18 nt, Artificial Sequence
  Description of Artificial Sequence Synthetic oligonucleotide
uggauaccguauaugcua